(19) United States
(12) Patent Application Publication
(10) Pub. No.: US 2017/0165247 A1
(43) Pub. Date: Jun. 15, 2017

(54) CRYSTALLINE COMPOUNDS OF DABIGATRAN ETEXILATE

(71) Applicant: OLON S.p.A., Rodano (MI) (IT)

(72) Inventors: Giorgio BERTOLINI, Rodano (MI) (IT); Lazzaro FELICIANI, Rodano (MI) (IT); Ilaria FERRANDO, Rodano (MI) (IT)

(21) Appl. No.: 15/327,138

(22) PCT Filed: Jul. 17, 2015
(86) PCT No.: PCT/IB2015/055436

§ 371 Date: Jan. 18, 2017

(30) Foreign Application Priority Data
Jul. 18, 2014 (IT) MI2014A001316

(51) Int. Cl. A61K 31/4439; A61K 31/19; A61K 9/14; C07D 401/12

(52) U.S. Cl. CPC A61K 31/4439; C07D 401/12; A61K 31/19; A61K 9/14

(57) ABSTRACT

The present invention relates to new crystalline compounds of dabigatran etexilate, namely to crystalline compounds comprising mixtures of dabigatran etexilate and an acid. The invention also relates to processes for the preparation of the new crystalline compounds, pharmaceutical compositions comprising them and their use in therapy.

### SUMMARY OF THE INVENTION

The present invention relates to new crystalline compounds of dabigatran etexilate, namely to crystalline compounds comprising mixtures of dabigatran etexilate and an acid. The invention also relates to processes for the preparation of the new crystalline compounds, pharmaceutical compositions comprising them and their use in therapy.

### TECHNICAL BACKGROUND

Dabigatran etexilate is the International Non Proprietary Name (INN) of 3-(((2-(((4-(N′-hexyloxicarbonyl-carbamidoyl)-phenyl)amino)methyl]-1-methyl-1H-benzimidazol-5-yl) carbonyl)-pyridin-2-yl-amino)-propionic acid ethyl ester of formula (I)

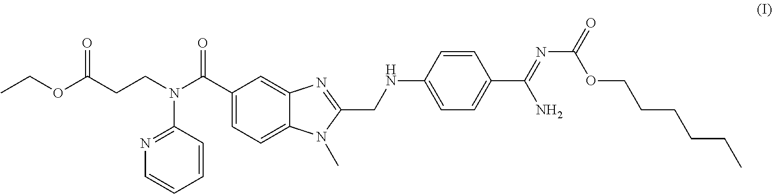 [SMILES: CCCCCCOC(=O)/N=C(\N)c1ccc(NCc2nc3cc(C(=O)N(CCC(=O)OCC)c4ccccn4)ccc3n2C)cc1]

Dabigatran etexilate is an innovative anticoagulant that acts inhibiting, directly and reversibly, thrombin, either when it is free and when it is bound to fibrin. As it is known, in the coagulation cascade thrombin enables the conversion of fibrinogen to fibrin and its inhibition prevents the formation of clots.

Dabigatran etexilate has poor solubility in water and is currently marketed as its mesylate salt under the trade name Pradaxa®.

This poor solubility leads to a consequent low bioavailability and variability of drug blood levels. Not being able to overcome these serious problems, particular formulations have been designed, such as those described in US2003/0181488, but these formulations require the application of a complex technology for the preparation of laborious multilayer compositions.

It is known that solid crystalline forms of active ingredients may show different physico-chemical properties and may offer advantages for example in terms of solubility, stability and bioavailability. Thus, the research and discovery of new crystalline forms of active pharmaceutical ingredients can lead to more reliable and effective therapies.

For this reason, it is considered a technical contribution to the art the preparation of new crystalline mixtures of active ingredients, since these new forms may allow an improved stability, bioavailability and pharmacokinetics, limit the hygroscopicity, and/or facilitate the galenic and industrial processing of active pharmaceutical ingredients.

But the preparation of said new crystal forms is not obvious, it is not predictable and is not always possible.

So, also for dabigatran etexilate, it is of interest to search for new crystalline forms which exhibit chemical and physical properties suitable for a safe and effective therapeutic use and that improve the solubility.

### OBJECTS OF THE INVENTION

It is an object of the invention to provide new crystalline compounds including dabigatran etexilate.

Another object of the invention to provide new crystalline compounds comprising dabigatran etexilate.

It is another object of the invention to provide new crystalline compounds comprising dabigatran etexilate, which are soluble, in particular equally or even more soluble the compound on the market, that is, dabigatran etexilate mesylate.

Another object of the invention to provide processes for the preparation of the said new crystalline compounds, pharmaceutical compositions containing them and their use in therapy.

### BRIEF DESCRIPTION OF THE DRAWINGS

FIG. 1 shows the XRPD of dabigatran etexilate acotinate anhydrous

FIG. 2 shows the FT-IR of dabigatran etexilate acotinate anhydrous

FIG. 3 shows the DSC of dabigatran etexilate acotinate anhydrous

FIG. 4 shows the XRPD of dabigatran etexilate adipate anhydrous

FIG. 5 shows the FT-IR of dabigatran etexilate adipate anhydrous

FIG. 6 shows the DSC of dabigatran etexilate adipate anhydrous

FIG. 7 shows the XRPD of dabigatran etexilate p-coumarate acetone solvate

FIG. 8 shows the XRPD of dabigatran etexilate p-coumarate acetone solvate

FIG. 9 shows the DSC of dabigatran etexilate p-coumarate acetone solvate

FIG. 10 shows the XRPD of dabigatran etexilate D-gluconate ethyl acetate solvate

FIG. 11 shows the FT-IR of dabigatran etexilate D-gluconate ethyl acetate solvate

FIG. 12 shows the DSC of dabigatran etexilate D-gluconate ethyl acetate solvate

FIG. 13 shows the XRPD of dabigatran etexilate α-chetoglutarate anhydrous

FIG. 14 shows the FT-IR of dabigatran etexilate α-chetoglutarate anhydrous

FIG. 15 shows the DSC of dabigatran etexilate α-chetoglutarate anhydrous

FIG. 16 shows the XRPD of dabigatran etexilate ippurate anhydrous Form A

FIG. 17 shows the FT-IR of dabigatran etexilate ippurate anhydrous Form A

FIG. 18 shows the DSC of dabigatran etexilate ippurate anhydrous Form A

FIG. 19 shows the XRPD of dabigatran etexilate itaconate hydrate

FIG. 20 shows the FT-IR of dabigatran etexilate itaconate hydrate

FIG. 21 shows the DSC of dabigatran etexilate itaconate hydrate

FIG. 22 shows the XRPD of dabigatran etexilate orotate hydrate Form B

FIG. 23 shows the FT-IR of dabigatran etexilate orotate hydrate Form B

FIG. 24 shows the DSC of dabigatran etexilate orotate hydrate Form B

FIG. 25 shows the XRPD of de dabigatran etexilate piruvate hydrate

FIG. 26 shows the FT-IR of de dabigatran etexilate piruvate hydrate

FIG. 27 shows the DSC of de dabigatran etexilate piruvate hydrate

FIG. 28 shows the XRPD of dabigatran etexilate sulfamate anhydrous

FIG. 29 shows the FT-IR of dabigatran etexilate sulfamate anhydrous

FIG. 30 shows the DSC of dabigatran etexilate sulfamate anhydrous

FIG. 31 shows the XRPD of dabigatran etexilate D-(−)-quinate anhydrous

FIG. 32 shows the FT-IR of dabigatran etexilate D-(−)-quinate anhydrous

FIG. 33 shows the DSC of dabigatran etexilate D-(−)-quinate anhydrous

FIG. 34 shows the XRPD of dabigatran etexilate ferulate anhydrous

FIG. 35 shows the FT-IR of dabigatran etexilate ferulate anhydrous

FIG. 36 shows the DSC of dabigatran etexilate ferulate anhydrous

FIG. 37 shows the XRPD of dabigatran etexilate gallate hydrate Form B

FIG. 38 shows the FT-IR of dabigatran etexilate gallate hydrate Form B

FIG. 39 shows the DSC of dabigatran etexilate gallate hydrate Form B

FIG. 40 shows the XRPD of dabigatran etexilate sebacate anhydrous

FIG. 41 shows the FT-IR of dabigatran etexilate sebacate anhydrous

FIG. 42 shows the DSC of dabigatran etexilate sebacate anhydrous

FIG. 43 shows the XRPD of dabigatran etexilate glutarate anhydrous

FIG. 44 shows the FT-IR of dabigatran etexilate glutarate anhydrous

FIG. 45 shows the DSC of dabigatran etexilate glutarate anhydrous

FIG. 46 shows the XRPD of dabigatran etexilate vanillate hydrate

FIG. 47 shows the FT-IR of dabigatran etexilate vanillate hydrate

FIG. 48 shows the DSC of dabigatran etexilate vanillate hydrate

FIG. 49 shows the XRPD of dabigatran etexilate caffeate hydrate Form A

FIG. 50 shows the FT-IR of dabigatran etexilate caffeate hydrate Form A

FIG. 51 shows the DSC of dabigatran etexilate caffeate hydrate Form A

FIG. 52 shows the XRPD of dabigatran etexilate caffeate hydrate Form B

FIG. 53 shows the XRPD of dabigatran etexilate ippurate hydrate Form B

FIG. 54 shows the XRPD of dabigatran etexilate gallate monohydrate Form A

FIG. 55 shows the FT-IR of dabigatran etexilate gallate monohydrate Form A

FIG. 56 shows the XRPD of dabigatran etexilate orotate anhydrous Form A

FIG. 57 shows the FT-IR of dabigatran etexilate orotate anhydrous Form A

FIG. 58 shows the kinetic dissolution of representative compounds of the invention (MES=mesylate salt; ORA=orotate salt; GLC=gallate salt; of dabigatran).

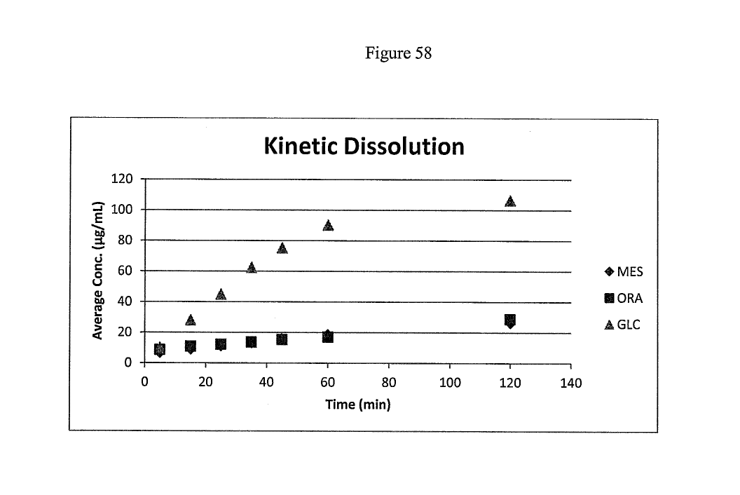

### DESCRIPTION OF THE INVENTION

It has now been found that it is possible to obtain new mixtures of compounds comprising dabigatran etexilate in a crystalline form.

In particular, it was found that certain mixtures of dabigatran etexilate with acids occur in a stable crystalline form and show chemical-physical properties suitable to their use in therapy. Some of these mixtures are crystal were also shown to be more soluble of the known compounds of dabigatran etexilate, in particular of its mesylate salt.

Thus, according to one of its aspects, the invention relates to a crystalline compound that comprises a mixture of dabigatran etexilate and a monocarboxylic acid selected from gallic acid, orotic acid, p-coumaric acid, hippuric acid, ferulic acid and vanillic acid, as well as hydrates and solvates thereof.

The crystalline compound which includes dabigatran etexilate and gallic acid is particularly preferred according to the invention.

The crystalline compound that includes dabigatran etexilate and orotic acid is also preferred according to the invention.

According to another of its aspects, the invention relates to a crystalline compound that comprises a mixture of dabigatran etexilate and an acid selected from aconitic acid, adipic acid, D-gluconic acid, α-cheto-glutaric acid, itaconic acid, pyruvic acid acid, sulfamic acid, D-quinico, sebacic acid, and glutaric acid, as well as hydrates and solvates thereof.

The anhydrous crystalline compounds as well as hydrates or solvates of all the above crystalline compounds, with water or other solvents, are a further subject-matter of the invention.

According to the present invention, the starting dabigatran etexilate may be dabigatran etexilate or a hydrated form of dabigatran etexilate preferably, but not necessary, dabigatran etexilate tetrahydrate.

By “crystalline compound” is meant here to indicate a mixture of dabigatran etexilate with one of the acids mentioned above, here also called “co-former”, said mixture having a crystalline form identifiable by X-ray diffraction.

The stoichiometry between the two components of the crystalline mixtures depends on the co-former used and/or the conditions of the process used.

According to another preferred embodiment, the invention relates to a crystalline compound dabigatran etexilate with gallic acid having the following formula

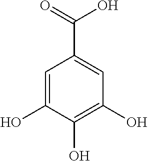 [SMILES: O=C(O)c1cc(O)c(O)c(O)c1]

advantageously monohydrate gallate dabigatran etexilate.

According to a preferred embodiment, the invention relates to a crystalline salt or a co-crystal of dabigatran etexilate with orotic acid having the following formula

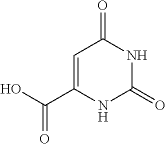 [SMILES: O=C(O)c1cc(=O)[nH]c(=O)[nH]1]

advantageously the anhydrous orotate dabigatran etexilate.

Gallate dabigatran etexilate, especially in the monohydrate form, which has a molar ratio of gallic acid/dabigatran equal to 1/1 is particularly preferred according to the invention, however, other molar ratios, for example 2/1 are however comprised within the scope of protection of the invention, as well as hydrates and solvates thereof.

Orotate dabigatran etexilate, especially in the anhydrous form, which has a molar ratio orotic acid/dabigatran equal to 1/1 is particularly preferred according to the invention, however, other molar ratios, for example 4/1 are however comprised within the scope of protection of the invention, as well as hydrates and solvates thereof.

Other crystalline compounds preferred according to the invention are selected from
anhydrous dabigatran etexilate aconitate;
anhydrous dabigatran etexilate adipate;
dabigatran etexilate p-cumarate acetone solvate;
dabigatran etexilate ethyl D-gluconate acetate solvate;
anhydrous α-keto-glutarate dabigatran etexilate;
anhydrous dabigatran etexilate hippurate;
dabigatran etexilate hydrate itaconate;
dabigatran etexilate hydrate pyruvate;
anhydrous sulfamate dabigatran etexilate;
anhydrous D-(−)-quinate dabigatran etexilate;
anhydrous dabigatran etexilate ferulate;
anhydrous dabigatran etexilate sebacate;
anhydrous dabigatran etexilate glutarate;
dabigatran etexilate vanillate hydrate.

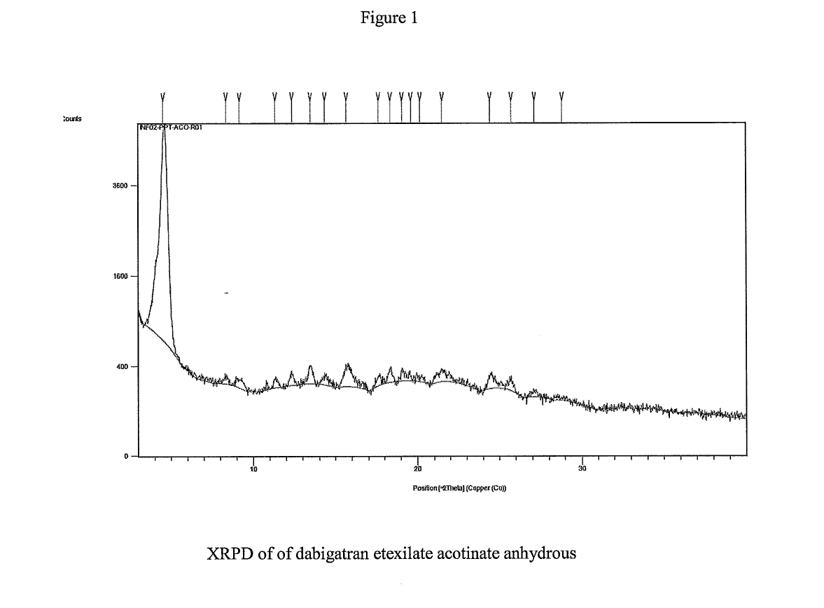

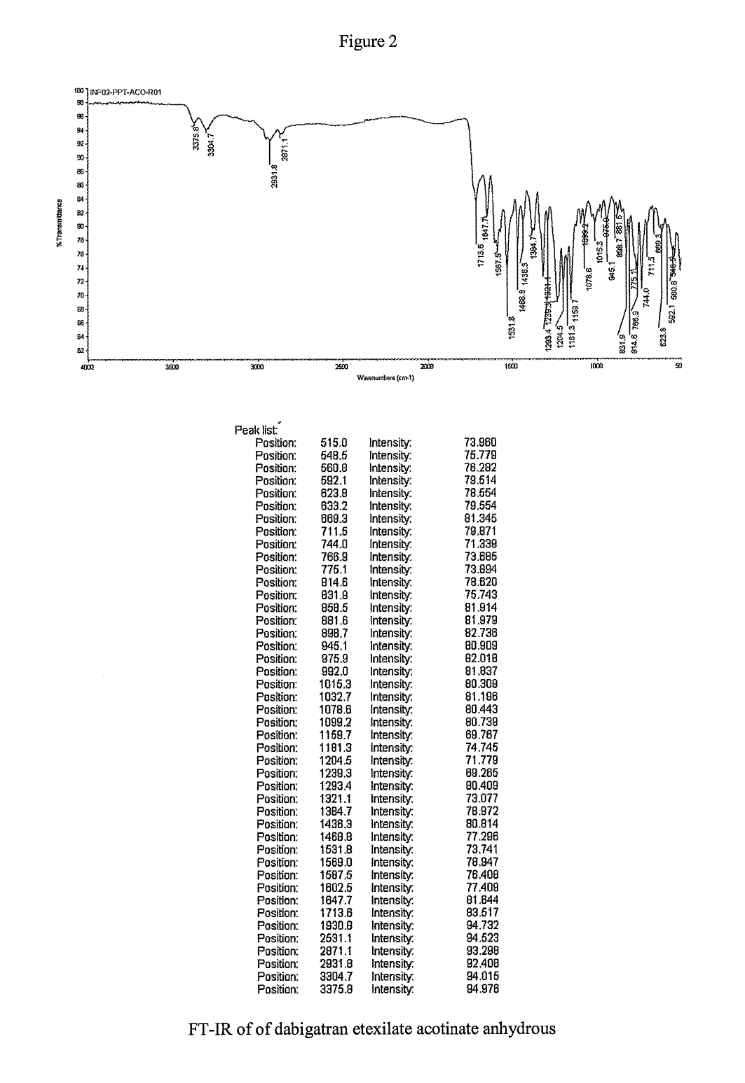

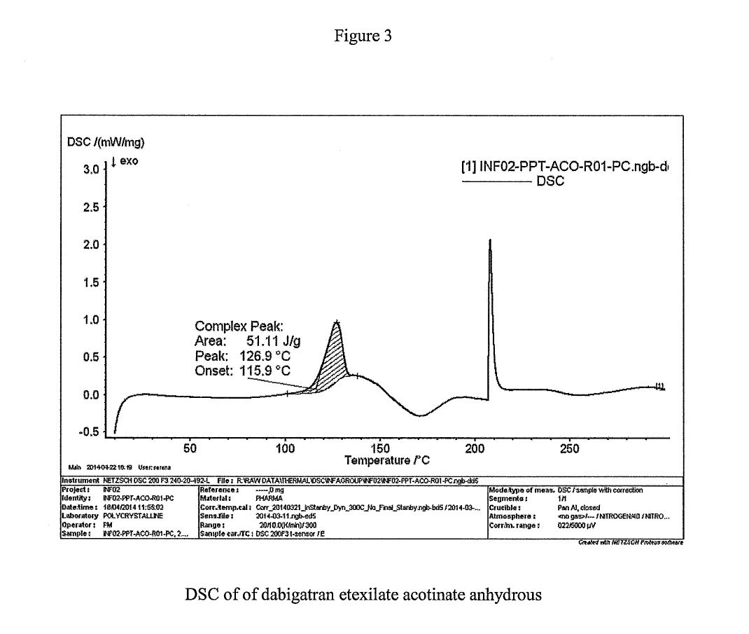

According to a preferred embodiment, the invention relates to anhydrous dabigatran etexilate aconitate showing the X-ray diffraction pattern of FIG. 1, the FT-IR spectrum of FIG. 2, the DSC profile of FIG. 3 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.5279 | 4387.75 | 0.2676 | 19.51594 | 100.00 |
| 8.3705 | 53.70 | 0.2007 | 10.56349 | 1.22 |
| 9.1819 | 63.75 | 0.4015 | 9.63171 | 1.45 |
| 11.3508 | 64.32 | 0.2676 | 7.79570 | 1.47 |
| 12.3592 | 81.41 | 0.2676 | 7.16182 | 1.86 |
| 13.4728 | 143.63 | 0.2676 | 6.57224 | 3.27 |
| 14.3707 | 74.75 | 0.2676 | 6.16358 | 1.70 |
| 15.6869 | 159.86 | 0.4015 | 5.64926 | 3.64 |
| 17.6400 | 71.98 | 0.3346 | 5.02792 | 1.64 |
| 18.3618 | 120.84 | 0.1004 | 4.83189 | 2.75 |
| 19.0655 | 99.85 | 0.1004 | 4.65509 | 2.28 |
| 19.6087 | 26.60 | 0.5353 | 4.52736 | 0.61 |
| 20.1616 | 30.70 | 0.2676 | 4.40444 | 0.70 |
| 21.5204 | 86.93 | 0.2007 | 4.12931 | 1.98 |
| 24.4349 | 100.34 | 0.3346 | 3.64298 | 2.29 |
| 25.7230 | 71.29 | 0.4015 | 3.46341 | 1.62 |
| 27.1615 | 31.50 | 0.4684 | 3.28316 | 0.72 |
| 28.8396 | 15.58 | 0.8029 | 3.09583 | 0.36 |

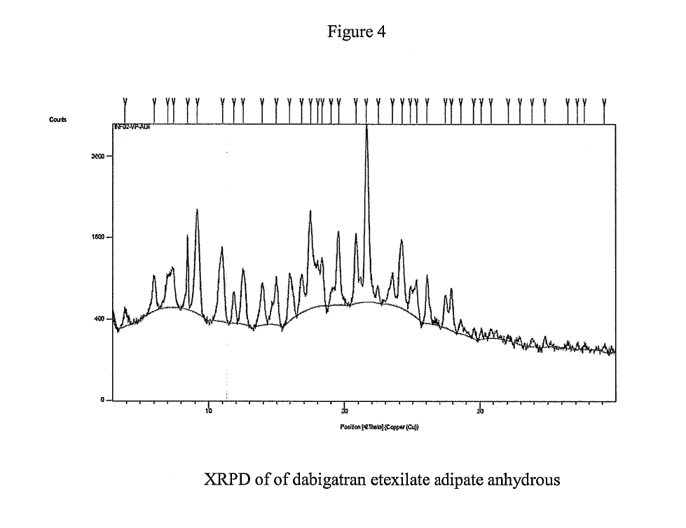

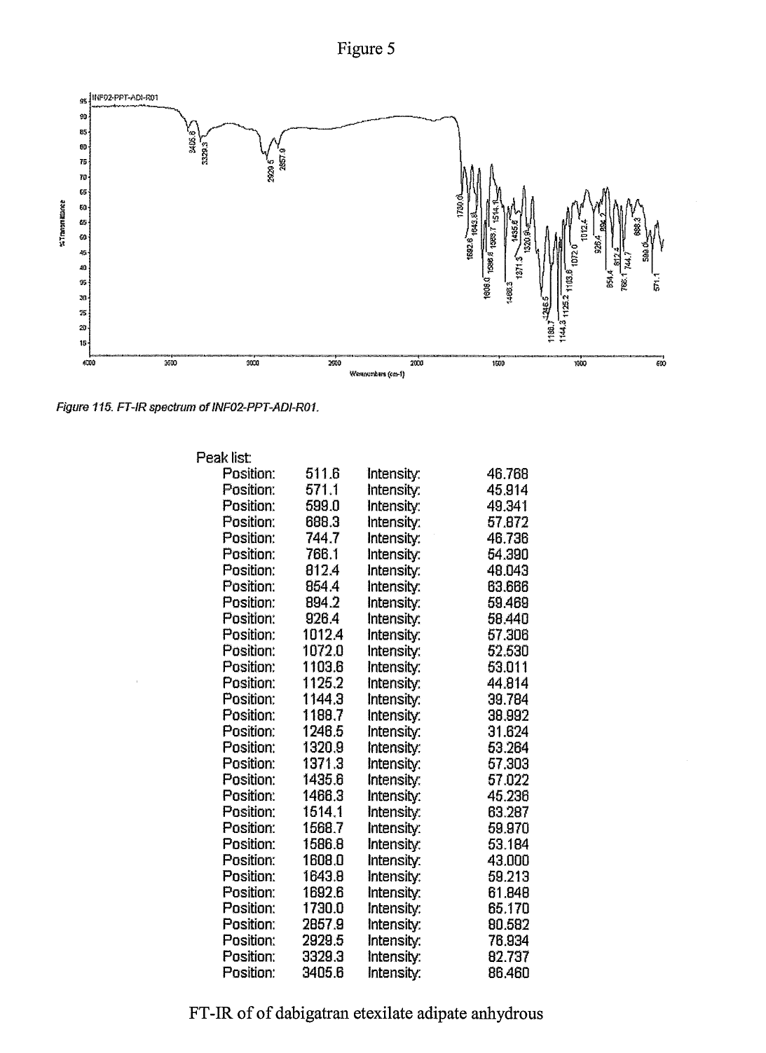

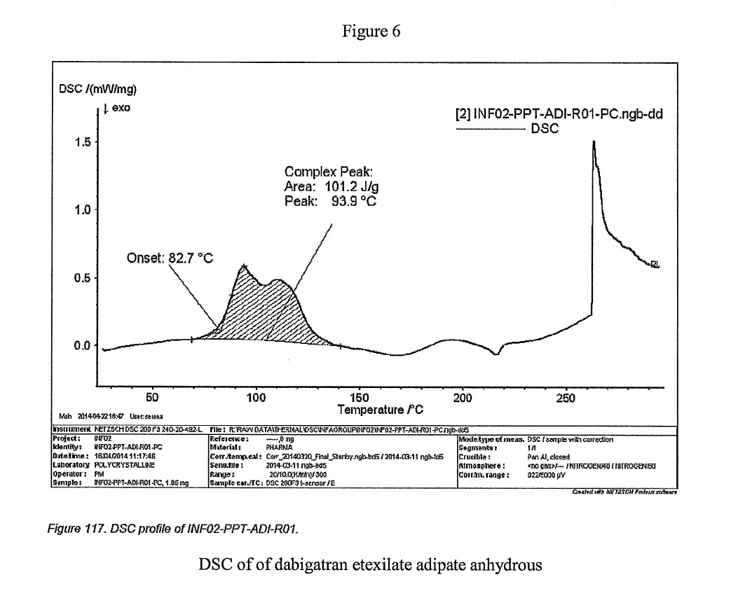

According to another preferred embodiment, the invention relates to anhydrous dabigatran etexilate adipate showing the X-ray diffraction pattern of FIG. 4, the FT-IR spectrum of FIG. 5, the DSC profile of FIG. 6 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 3.9026 | 150.46 | 0.2007 | 22.64148 | 3.93 |
| 6.0261 | 414.89 | 0.2342 | 14.66691 | 10.85 |
| 6.9935 | 406.28 | 0.1673 | 12.64003 | 10.63 |
| 7.4307 | 506.28 | 0.2007 | 11.89723 | 13.24 |
| 8.4609 | 1157.63 | 0.1004 | 10.45078 | 30.28 |
| 9.1792 | 1733.06 | 0.1506 | 9.63455 | 45.33 |
| 11.0139 | 973.88 | 0.1004 | 8.03338 | 25.47 |
| 11.8511 | 346.29 | 0.2007 | 7.46770 | 9.06 |
| 12.5035 | 670.38 | 0.3346 | 7.07948 | 17.53 |
| 13.9430 | 477.80 | 0.2342 | 6.35168 | 12.50 |
| 14.9901 | 547.77 | 0.0669 | 5.91027 | 14.33 |
| 15.9406 | 549.92 | 0.2676 | 5.55991 | 14.38 |
| 16.8429 | 472.77 | 0.2342 | 5.26404 | 12.36 |
| 17.4839 | 1640.48 | 0.0836 | 5.07246 | 42.90 |
| 18.0211 | 611.87 | 0.1004 | 4.92245 | 16.00 |
| 18.3486 | 679.63 | 0.1673 | 4.83533 | 17.77 |
| 19.0043 | 163.83 | 0.2007 | 4.66996 | 4.28 |
| 19.5662 | 1113.06 | 0.1673 | 4.53708 | 29.11 |
| 20.8014 | 1064.49 | 0.0669 | 4.27039 | 27.84 |
| 21.5729 | 3823.59 | 0.1673 | 4.11938 | 100.00 |
| 22.4527 | 187.42 | 0.1171 | 3.95991 | 4.90 |
| 23.5033 | 448.61 | 0.1004 | 3.78522 | 11.73 |
| 24.2210 | 968.18 | 0.1338 | 3.67467 | 25.32 |
| 24.8065 | 313.61 | 0.1338 | 3.58925 | 8.20 |
| 25.2475 | 469.32 | 0.1004 | 3.52754 | 12.27 |
| 26.0293 | 596.44 | 0.0836 | 3.42335 | 15.60 |
| 27.4276 | 351.40 | 0.2007 | 3.25191 | 9.19 |
| 27.8841 | 469.49 | 0.0836 | 3.19970 | 12.28 |
| 28.5841 | 110.10 | 0.1338 | 3.12292 | 2.88 |
| 29.5458 | 71.29 | 0.2342 | 3.02342 | 1.86 |
| 30.0984 | 80.71 | 0.1338 | 2.96915 | 2.11 |
| 30.8098 | 73.14 | 0.1673 | 2.90220 | 1.91 |
| 32.1040 | 26.38 | 0.2676 | 2.78810 | 0.69 |
| 32.9513 | 47.00 | 0.1673 | 2.71832 | 1.23 |
| 33.8588 | 51.97 | 0.3346 | 2.64751 | 1.36 |
| 34.7580 | 70.10 | 0.1338 | 2.58105 | 1.83 |
| 36.4467 | 31.27 | 0.4015 | 2.46524 | 0.82 |
| 37.1413 | 28.67 | 0.1673 | 2.42072 | 0.75 |
| 37.6967 | 29.59 | 0.2676 | 2.38632 | 0.77 |
| 39.1255 | 39.10 | 0.2007 | 2.30241 | 1.02 |

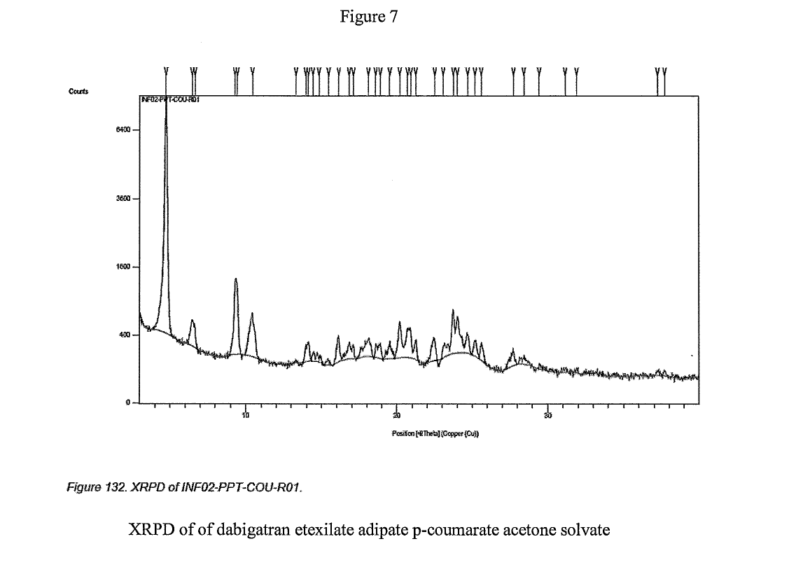

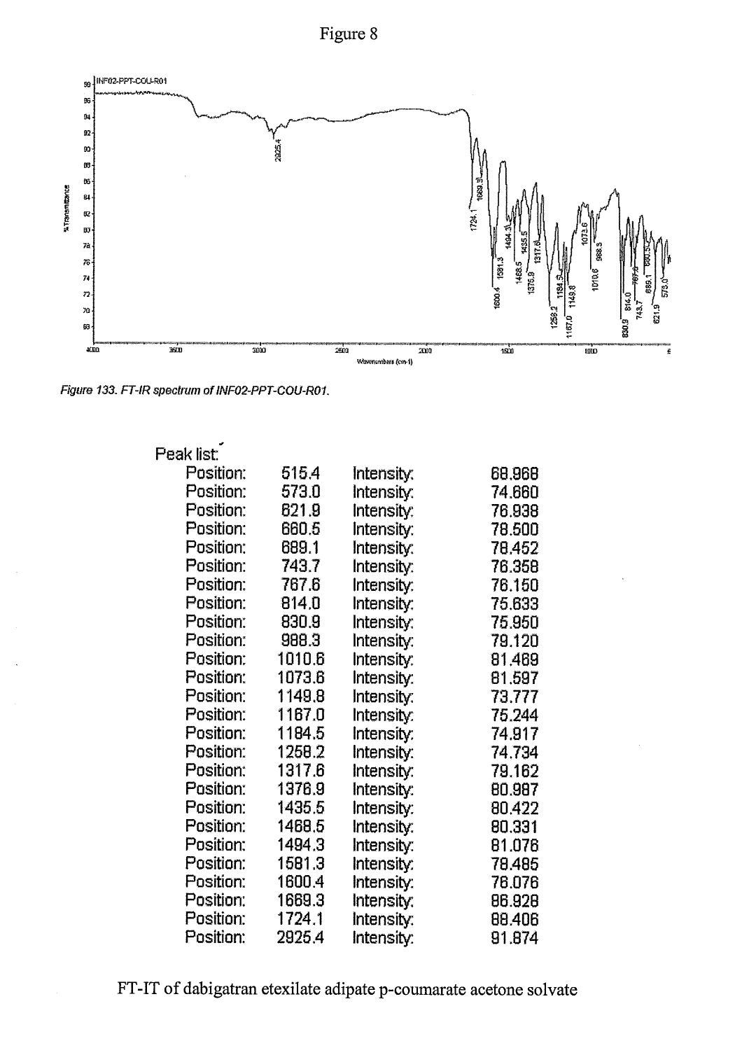

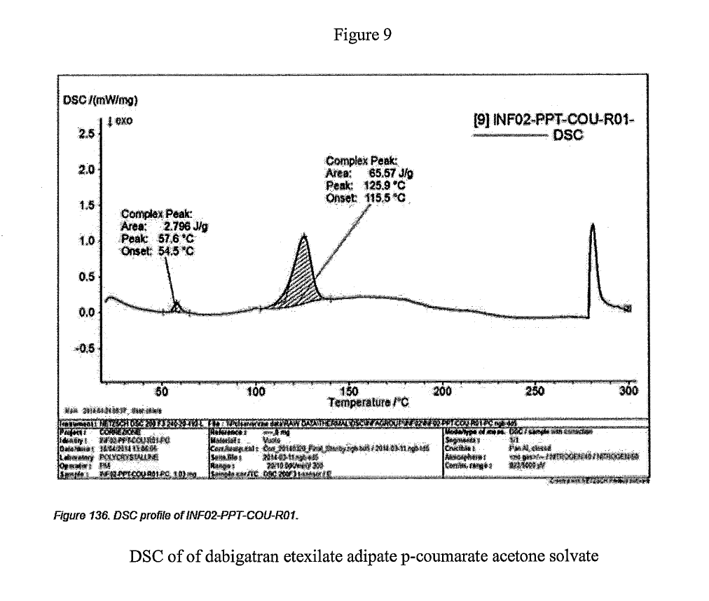

According to another preferred embodiment, the invention relates to dabigatran etexilate coumarate acetone solvate showing the X-ray diffraction pattern of FIG. 7, the FT-IR spectrum of FIG. 8, the DSC profile of FIG. 9 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.7376 | 7748.40 | 0.1673 | 18.65248 | 100.00 |
| 6.4860 | 315.03 | 0.1673 | 13.62788 | 4.07 |
| 6.6605 | 248.22 | 0.0502 | 13.27126 | 3.20 |
| 9.3152 | 1014.64 | 0.1171 | 9.49423 | 13.09 |
| 9.4677 | 860.22 | 0.1171 | 9.34161 | 11.10 |
| 10.4486 | 516.41 | 0.0669 | 8.46672 | 6.66 |
| 13.3185 | 15.58 | 0.2007 | 6.64807 | 0.20 |
| 13.9559 | 133.77 | 0.1004 | 6.34581 | 1.73 |
| 14.1509 | 161.73 | 0.1004 | 6.25882 | 2.09 |
| 14.4436 | 61.87 | 0.1338 | 6.13263 | 0.80 |
| 14.8566 | 34.91 | 0.2007 | 5.96306 | 0.45 |
| 15.4795 | 25.17 | 0.2342 | 5.72449 | 0.32 |
| 16.1435 | 236.87 | 0.1171 | 5.49051 | 3.06 |
| 16.8329 | 144.43 | 0.1338 | 5.26716 | 1.86 |
| 17.1197 | 98.05 | 0.0836 | 5.17956 | 1.27 |
| 18.1154 | 167.24 | 0.1673 | 4.89705 | 2.16 |
| 18.5986 | 118.58 | 0.0502 | 4.77089 | 1.53 |
| 18.8826 | 121.47 | 0.1338 | 4.69978 | 1.57 |
| 19.5233 | 139.21 | 0.1338 | 4.54695 | 1.80 |
| 20.1805 | 394.79 | 0.1506 | 4.40035 | 5.10 |
| 20.6925 | 298.84 | 0.1338 | 4.29261 | 3.86 |
| 20.9051 | 256.20 | 0.1004 | 4.24943 | 3.31 |
| 21.2455 | 184.48 | 0.1171 | 4.18211 | 2.38 |
| 22.5040 | 164.56 | 0.1171 | 3.95100 | 2.12 |
| 23.0773 | 104.95 | 0.1673 | 3.85413 | 1.35 |
| 23.7460 | 525.55 | 0.1171 | 3.74709 | 6.78 |
| 24.0019 | 430.44 | 0.1004 | 3.70771 | 5.56 |
| 24.6941 | 187.16 | 0.1673 | 3.60533 | 2.42 |
| 25.1872 | 153.78 | 0.2007 | 3.53585 | 1.98 |
| 25.5792 | 143.62 | 0.1673 | 3.48254 | 1.85 |
| 27.7398 | 109.52 | 0.0836 | 3.21602 | 1.41 |
| 28.4302 | 45.02 | 0.4015 | 3.13947 | 0.58 |
| 29.4516 | 22.06 | 0.2007 | 3.03288 | 0.28 |
| 31.1630 | 16.11 | 0.3346 | 2.87011 | 0.21 |
| 31.9226 | 17.67 | 0.2007 | 2.80353 | 0.23 |
| 37.2617 | 22.05 | 0.2007 | 2.41318 | 0.28 |
| 37.7099 | 25.85 | 0.2007 | 2.38552 | 0.33 |

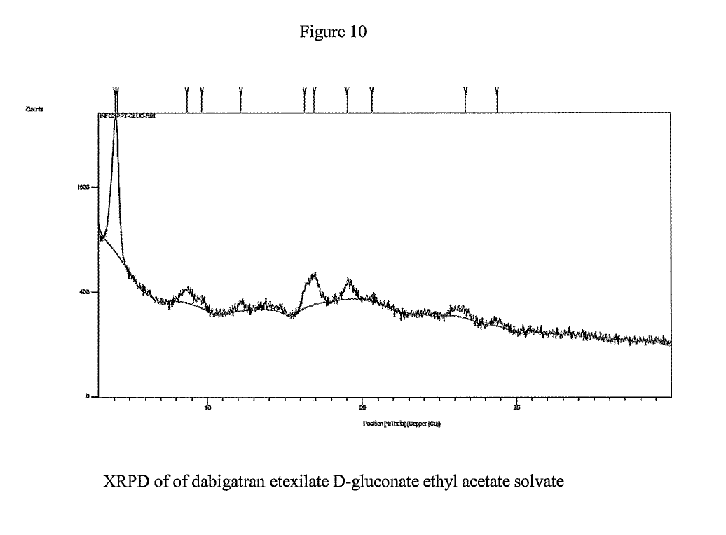

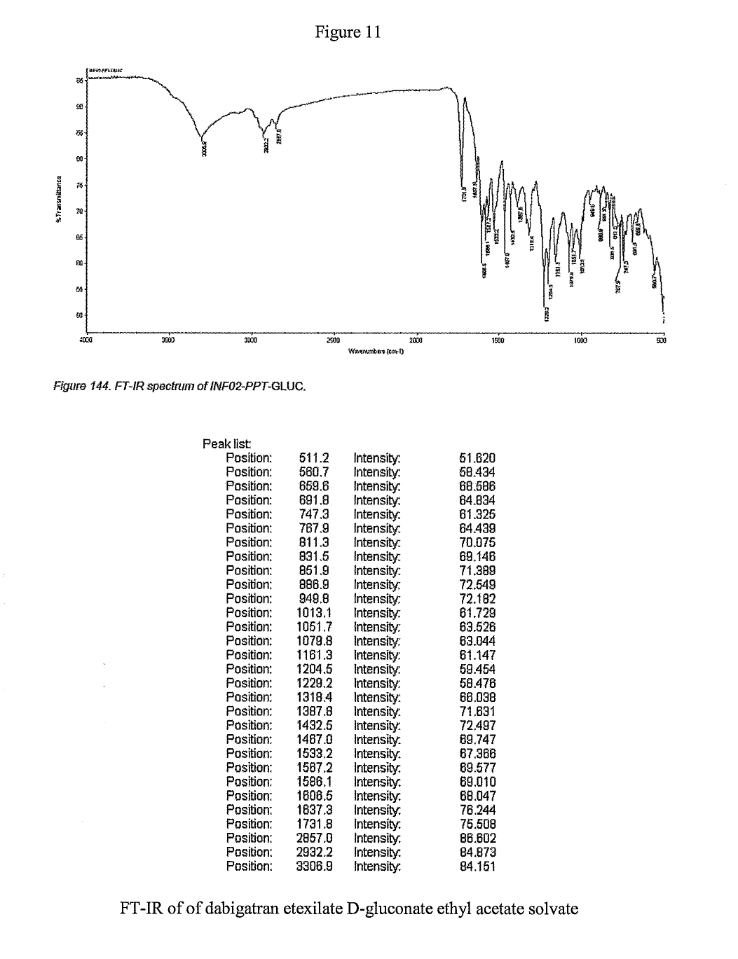

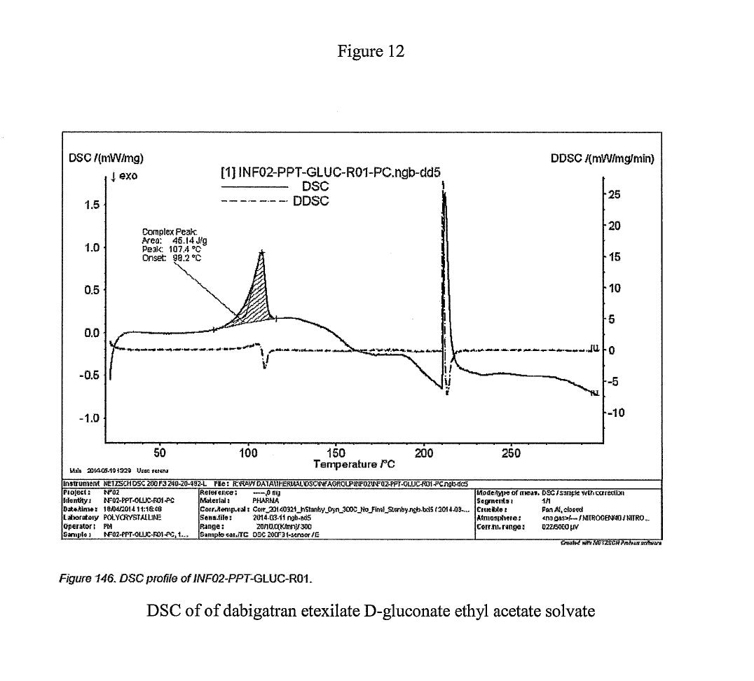

According to another preferred embodiment, the invention relates to dabigatran etexilate gluconate acetate solvate showing the X-ray diffraction pattern of FIG. 10, the FT-IR spectrum of FIG. 11, the DSC profile of FIG. 12 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.0512 | 2072.30 | 0.1171 | 21.81095 | 100.00 |
| 4.1866 | 1796.54 | 0.0836 | 21.10581 | 86.69 |
| 8.6693 | 100.07 | 0.4684 | 10.20005 | 4.83 |
| 9.6597 | 60.32 | 0.4015 | 9.15635 | 2.91 |
| 12.1762 | 59.78 | 0.2676 | 7.26907 | 2.88 |
| 16.2940 | 134.87 | 0.2676 | 5.44011 | 6.51 |
| 16.9028 | 222.13 | 0.4684 | 5.24553 | 10.72 |
| 19.0274 | 140.10 | 0.3346 | 4.66433 | 6.76 |
| 20.6159 | 27.12 | 0.5353 | 4.30837 | 1.31 |
| 26.7452 | 45.99 | 0.6691 | 3.33331 | 2.22 |
| 28.7621 | 28.78 | 0.6691 | 3.10399 | 1.39 |

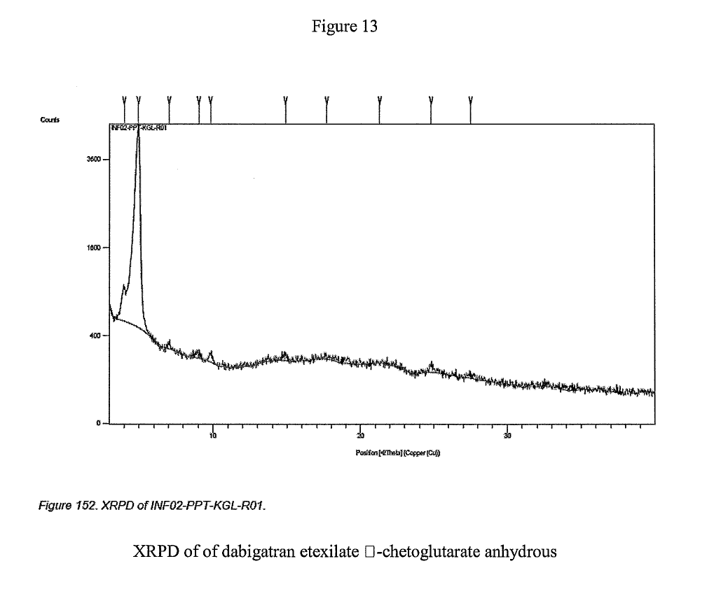

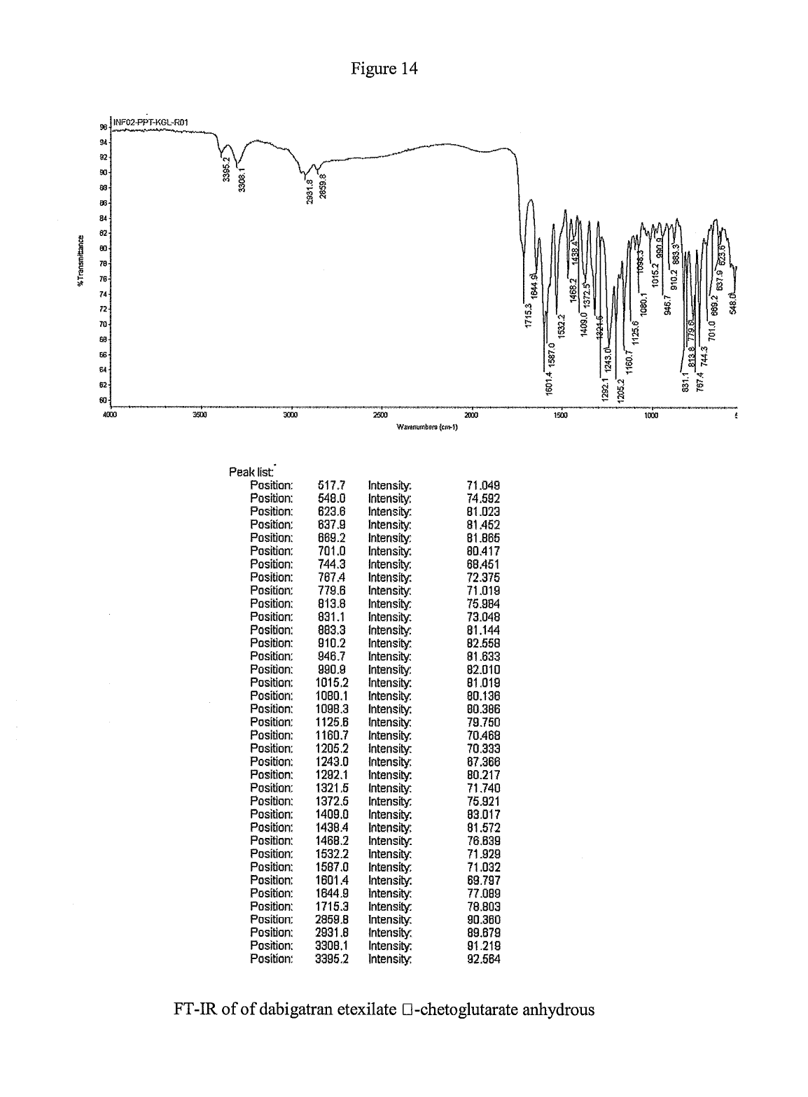

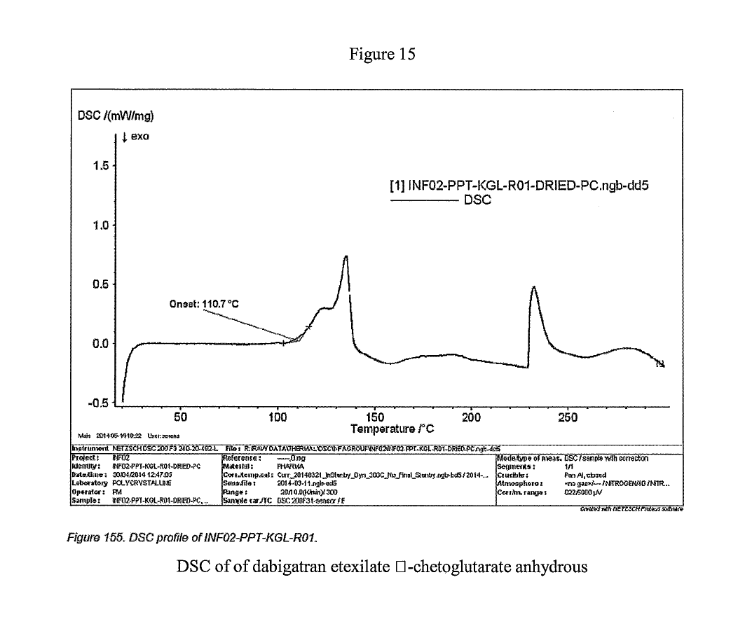

According to another preferred embodiment, the invention relates to anhydrous dabigatran etexilate α-ketoglutarate showing the X-ray diffraction pattern of FIG. 13, the FT-IR spectrum of FIG. 14, the DSC profile of FIG. 15 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 3.9837 | 426.54 | 0.1338 | 22.18053 | 10.21 |
| 4.9531 | 4178.95 | 0.1673 | 17.84149 | 100.00 |
| 7.0434 | 46.06 | 0.2007 | 12.55057 | 1.10 |
| 9.0460 | 31.96 | 0.4015 | 9.77616 | 0.76 |
| 9.8424 | 60.97 | 0.2007 | 8.98676 | 1.46 |
| 14.8935 | 40.87 | 0.2676 | 5.94836 | 0.98 |
| 17.6977 | 19.41 | 0.8029 | 5.01168 | 0.46 |
| 21.3215 | 5.99 | 0.8029 | 4.16738 | 0.14 |
| 24.8289 | 38.69 | 0.2007 | 3.58605 | 0.93 |
| 27.5037 | 16.97 | 0.5353 | 3.24309 | 0.41 |

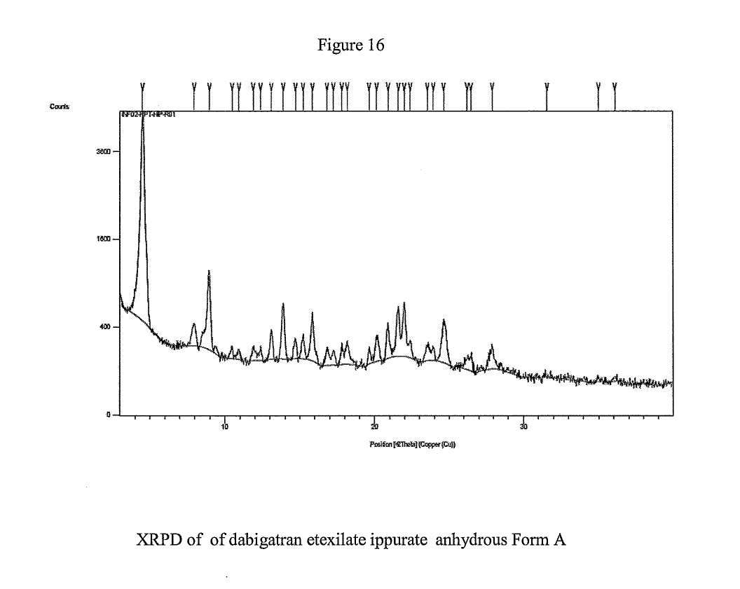

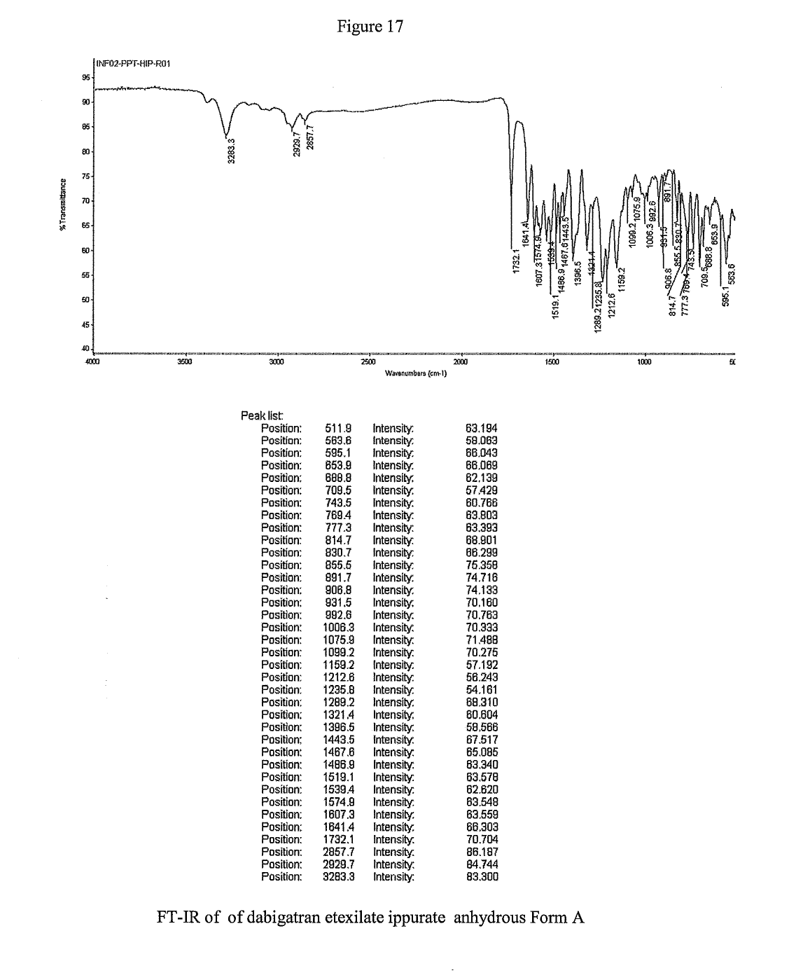

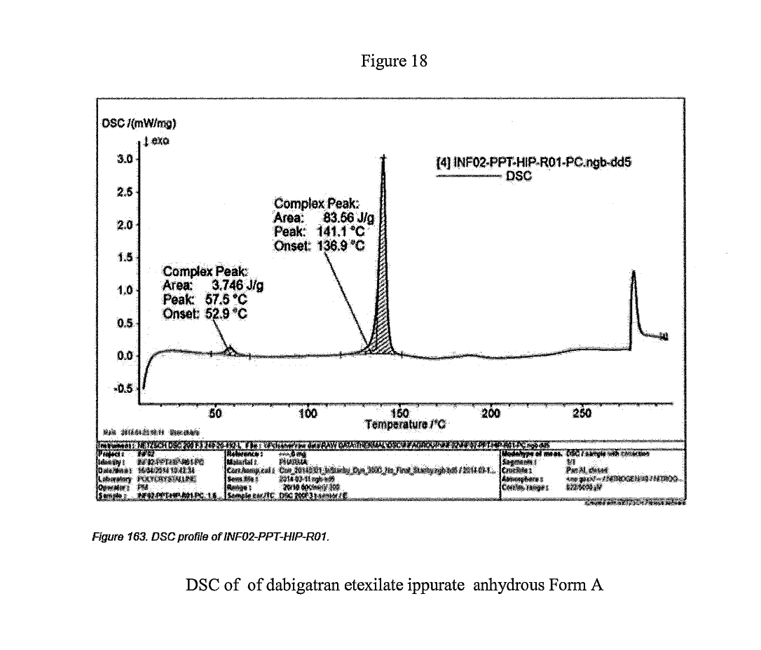

According to another preferred embodiment, the invention relates to anhydrous dabigatran etexilate ippurate, Form A, showing the X-ray diffraction pattern of FIG. 16, the FT-IR spectrum of FIG. 17, the DSC profile of FIG. 18 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.5086 | 4375.81 | 0.1673 | 19.59932 | 100.00 |
| 7.9552 | 175.93 | 0.3346 | 11.11391 | 4.02 |
| 8.9753 | 838.61 | 0.1338 | 9.85298 | 19.16 |
| 10.4990 | 74.99 | 0.1004 | 8.42619 | 1.71 |
| 10.9475 | 59.51 | 0.1673 | 8.08197 | 1.36 |
| 11.9073 | 83.82 | 0.1004 | 7.43260 | 1.92 |
| 12.3957 | 80.64 | 0.1338 | 7.14084 | 1.84 |
| 13.0846 | 203.85 | 0.1171 | 6.76634 | 4.66 |
| 13.9190 | 451.18 | 0.1506 | 6.36256 | 10.31 |
| 14.7250 | 131.51 | 0.2007 | 6.01607 | 3.01 |
| 15.2232 | 160.96 | 0.1673 | 5.82030 | 3.68 |
| 15.8594 | 402.35 | 0.0669 | 5.58821 | 9.19 |
| 16.8446 | 97.23 | 0.1673 | 5.26352 | 2.22 |
| 17.2683 | 84.82 | 0.2007 | 5.13531 | 1.94 |
| 17.8375 | 95.09 | 0.1338 | 4.97270 | 2.17 |
| 18.1851 | 135.87 | 0.2007 | 4.87844 | 3.11 |
| 19.6391 | 85.21 | 0.1338 | 4.52042 | 1.95 |
| 20.1453 | 164.69 | 0.2342 | 4.40796 | 3.76 |
| 20.8911 | 232.68 | 0.0669 | 4.25225 | 5.32 |
| 21.5942 | 387.09 | 0.0836 | 4.11537 | 8.85 |
| 21.9641 | 482.52 | 0.1004 | 4.04689 | 11.03 |
| 22.3824 | 121.39 | 0.1338 | 3.97218 | 2.77 |
| 23.5171 | 92.09 | 0.2007 | 3.78304 | 2.10 |
| 23.9283 | 80.77 | 0.1338 | 3.71895 | 1.85 |
| 24.6395 | 313.76 | 0.2676 | 3.61319 | 7.17 |
| 26.2085 | 55.18 | 0.5353 | 3.40034 | 1.26 |
| 26.4820 | 81.36 | 0.1004 | 3.36584 | 1.86 |
| 27.9185 | 130.26 | 0.1338 | 3.19583 | 2.98 |
| 31.6045 | 4.43 | 0.5353 | 2.83101 | 0.10 |
| 35.0282 | 10.93 | 0.4015 | 2.56176 | 0.25 |
| 36.1184 | 13.30 | 0.2676 | 2.48690 | 0.30 |

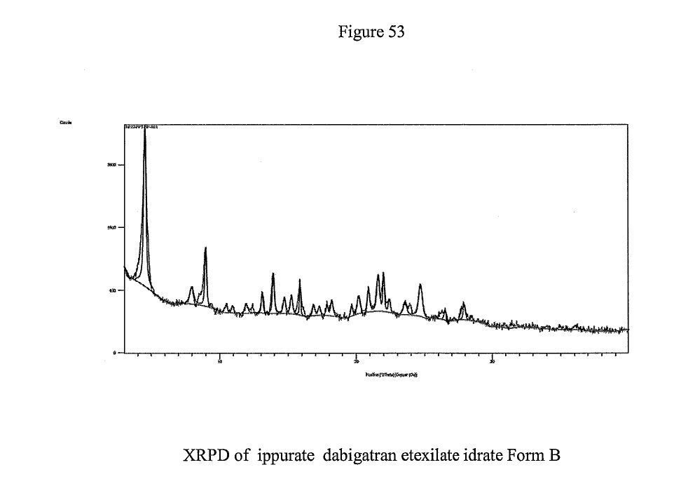

According to another preferred embodiment, the invention relates to dabigatran etexilate hippurate, Form B, obtained by vapour digestion, showing the X-ray diffraction pattern of FIG. 53, and following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.3416 | 5685.21 | 0.1673 | 20.35270 | 100.00 |
| 7.4845 | 542.10 | 0.1171 | 11.81183 | 9.54 |
| 8.0593 | 754.28 | 0.2007 | 10.97070 | 13.27 |
| 8.6386 | 521.72 | 0.2342 | 10.23627 | 9.18 |
| 9.1840 | 285.99 | 0.1673 | 9.62950 | 5.03 |
| 10.5097 | 280.73 | 0.2007 | 8.41766 | 4.94 |
| 11.0636 | 103.41 | 0.2342 | 7.99739 | 1.82 |
| 12.3178 | 284.09 | 0.3011 | 7.18581 | 5.00 |
| 13.9061 | 1163.91 | 0.1171 | 6.36846 | 20.47 |
| 14.9584 | 755.51 | 0.1004 | 5.92273 | 13.29 |
| 16.0965 | 302.74 | 0.1673 | 5.50643 | 5.33 |
| 16.6933 | 243.25 | 0.3680 | 5.31090 | 4.28 |
| 17.7250 | 192.58 | 0.1673 | 5.00401 | 3.39 |
| 18.3204 | 478.17 | 0.2342 | 4.84270 | 8.41 |
| 19.0641 | 71.70 | 0.2676 | 4.65542 | 1.26 |
| 20.8941 | 236.86 | 0.2007 | 4.25165 | 4.17 |
| 21.7590 | 1415.43 | 0.1506 | 4.08456 | 24.90 |
| 23.8244 | 268.98 | 0.1338 | 3.73494 | 4.73 |
| 24.9454 | 189.38 | 0.4015 | 3.56957 | 3.33 |
| 26.5028 | 152.53 | 0.2342 | 3.36325 | 2.68 |
| 28.0346 | 134.42 | 0.4015 | 3.18286 | 2.36 |
| 28.9580 | 116.40 | 0.4015 | 3.08344 | 2.05 |
| 30.2562 | 39.45 | 0.5353 | 2.95403 | 0.69 |

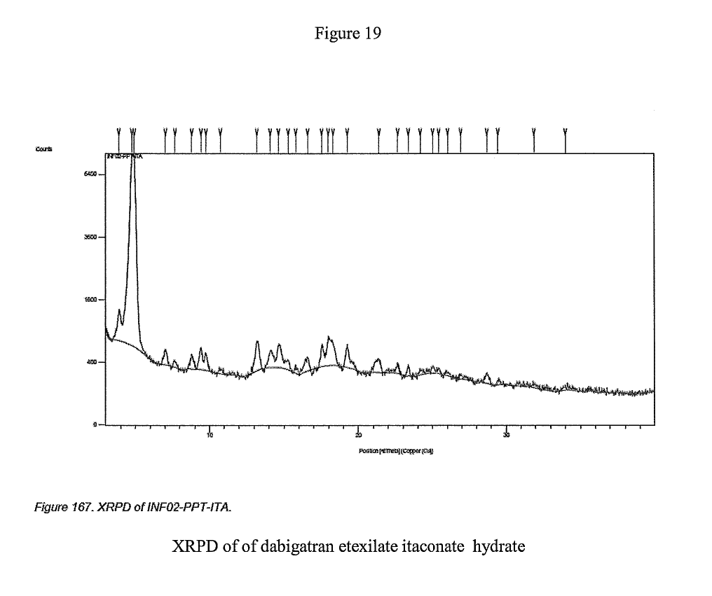

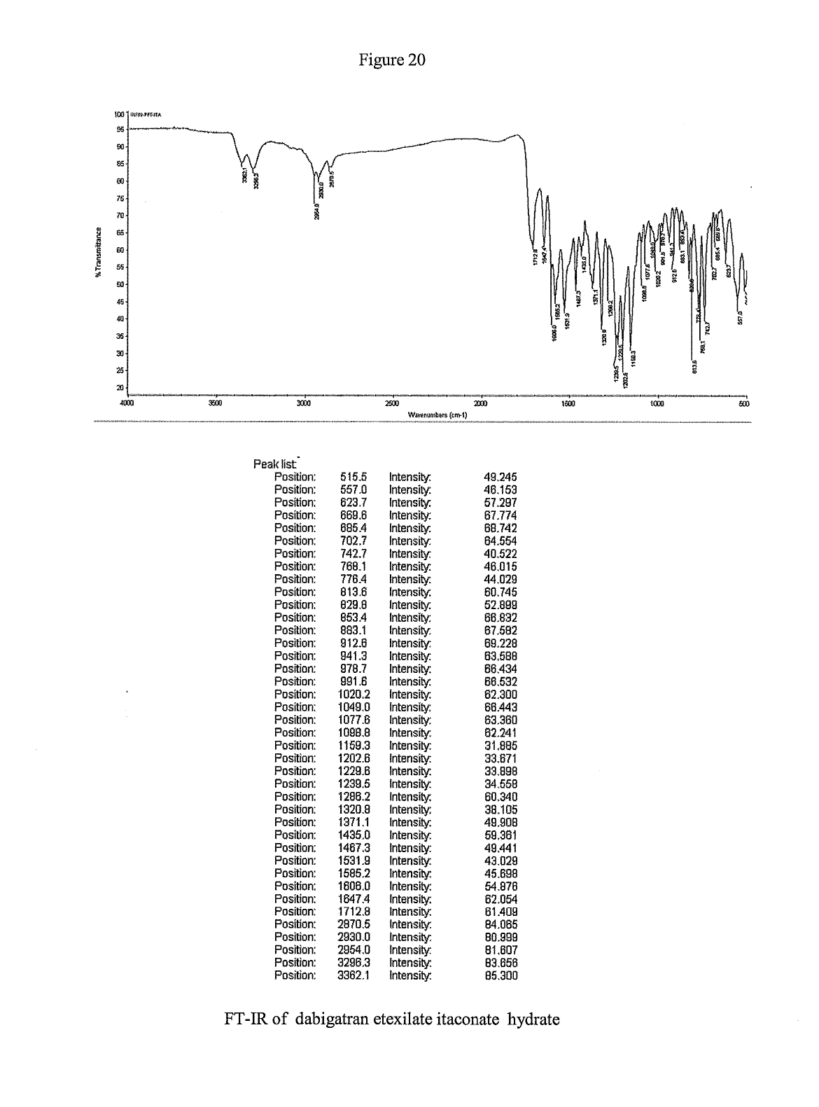

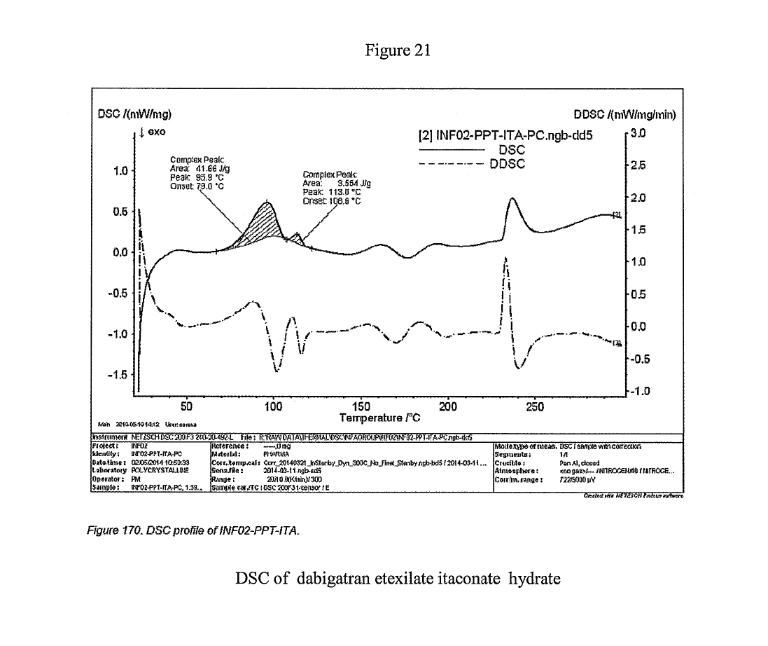

According to another preferred embodiment, the invention relates to dabigatran etexilate itaconate hydrate showing the X-ray diffraction pattern of FIG. 19, the FT-IR spectrum of FIG. 20, the DSC profile of FIG. 21 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 3.8775 | 601.91 | 0.1840 | 22.78769 | 8.76 |
| 4.7549 | 6867.64 | 0.1338 | 18.58473 | 100.00 |
| 4.9074 | 6681.57 | 0.1673 | 18.00761 | 97.29 |
| 7.0357 | 212.65 | 0.1673 | 12.56435 | 3.10 |
| 7.6675 | 76.37 | 0.2007 | 11.53029 | 1.11 |
| 8.8006 | 185.41 | 0.1338 | 10.04812 | 2.70 |
| 9.4291 | 300.57 | 0.1171 | 9.37980 | 4.38 |
| 9.7575 | 213.86 | 0.1673 | 9.06475 | 3.11 |
| 10.7474 | 49.32 | 0.2676 | 8.23197 | 0.72 |
| 13.1860 | 420.30 | 0.1171 | 6.71456 | 6.12 |
| 14.0812 | 221.37 | 0.2007 | 6.28961 | 3.22 |
| 14.6308 | 314.69 | 0.2342 | 6.05460 | 4.58 |
| 15.2869 | 115.06 | 0.1673 | 5.79618 | 1.68 |
| 15.8038 | 65.59 | 0.1338 | 5.60774 | 0.96 |
| 16.6059 | 150.09 | 0.1004 | 5.33863 | 2.19 |
| 17.5283 | 294.47 | 0.0836 | 5.05971 | 4.29 |
| 17.9850 | 403.22 | 0.1673 | 4.93225 | 5.87 |
| 18.3208 | 305.67 | 0.2007 | 4.84260 | 4.45 |
| 19.2574 | 308.53 | 0.1673 | 4.60913 | 4.49 |
| 21.4051 | 141.57 | 0.1673 | 4.15129 | 2.06 |
| 22.6621 | 98.46 | 0.1338 | 3.92380 | 1.43 |
| 23.3927 | 95.25 | 0.1171 | 3.80287 | 1.39 |

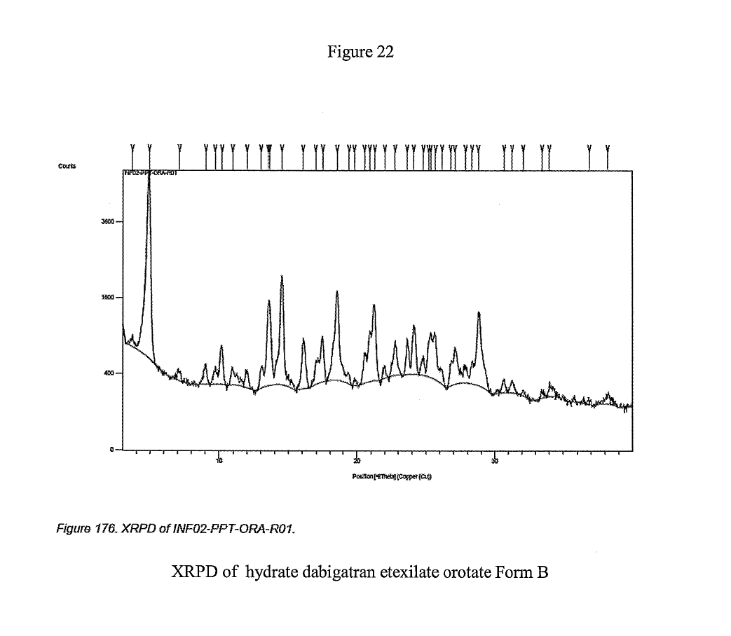

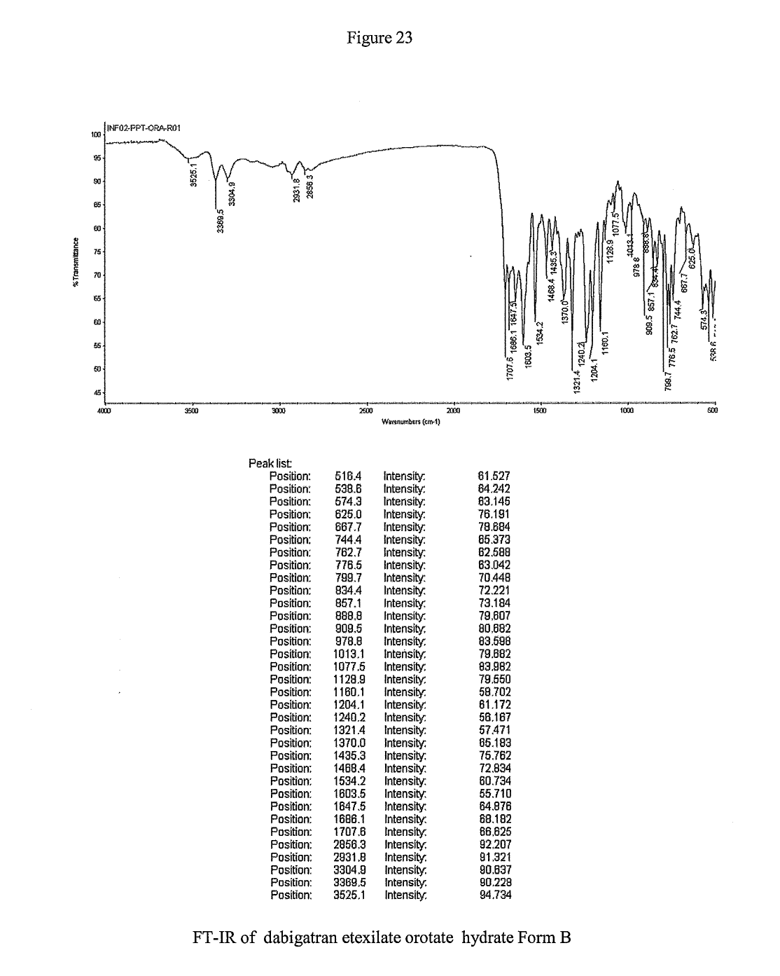

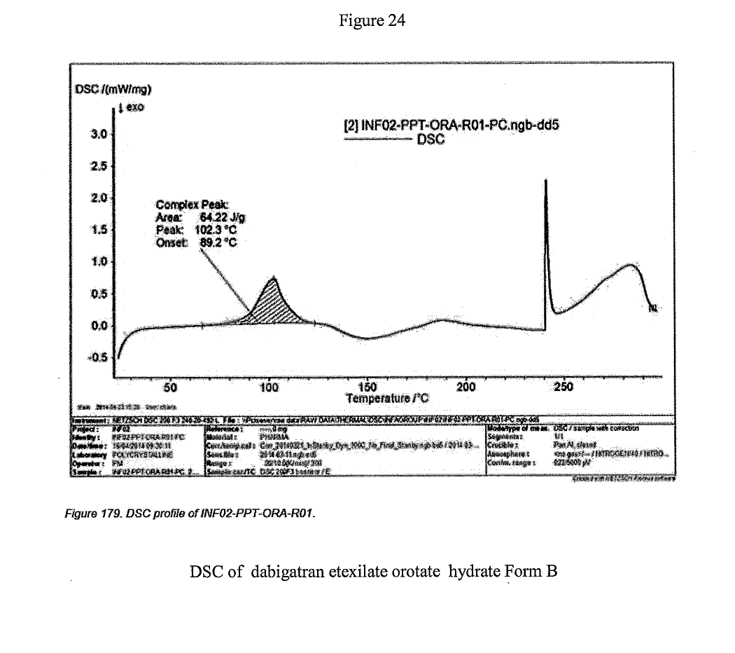

According to another preferred embodiment, the invention relates to dabigatran etexilate orotate hydrate Form B (ratio dabigatran/orotate 1/4) showing the X-ray diffraction pattern of FIG. 22, the FT-IR spectrum of FIG. 23, the DSC profile of FIG. 24 and the following characteristics of X-ray:

| Pos. | Height | FWHM | d-spacing | Rel. Int. |
|---|---|---|---|---|
| [°2Th.] | [cts] | [°2Th.] | [Å] | [%] |
| 3.7023 | 131.45 | 0.2676 | 23.86592 | 2.90 |
| 4.9437 | 4535.80 | 0.2007 | 17.87549 | 100.00 |
| 7.1173 | 92.88 | 0.2676 | 12.42042 | 2.05 |
| 9.0366 | 191.03 | 0.2007 | 9.78630 | 4.21 |
| 9.6897 | 135.01 | 0.1338 | 9.12811 | 2.98 |
| 10.1967 | 438.24 | 0.1840 | 8.67527 | 9.66 |
| 10.9491 | 176.69 | 0.1673 | 8.08079 | 3.90 |
| 12.0068 | 162.28 | 0.2342 | 7.37121 | 3.58 |
| 13.0267 | 194.81 | 0.2342 | 6.79633 | 4.29 |
| 13.5286 | 1106.52 | 0.0669 | 6.54527 | 24.40 |
| 13.6197 | 1255.21 | 0.1171 | 6.50171 | 27.67 |
| 14.5138 | 1813.50 | 0.2007 | 6.10312 | 39.98 |
| 16.0484 | 537.15 | 0.0669 | 5.52281 | 11.84 |
| 16.9681 | 229.02 | 0.1338 | 5.22548 | 5.05 |
| 17.4938 | 527.50 | 0.1673 | 5.06964 | 11.63 |
| 18.5263 | 1419.37 | 0.2007 | 4.78934 | 31.29 |
| 19.3994 | 98.49 | 0.1673 | 4.57571 | 2.17 |
| 19.7933 | 47.18 | 0.1673 | 4.48554 | 1.04 |
| 20.5322 | 316.18 | 0.1673 | 4.32576 | 6.97 |
| 20.8923 | 618.15 | 0.1338 | 4.25200 | 13.63 |
| 21.2837 | 1010.92 | 0.1840 | 4.17469 | 22.29 |
| 21.9983 | 118.03 | 0.2342 | 4.04067 | 2.60 |
| 22.7673 | 392.96 | 0.0836 | 3.90590 | 8.66 |
| 23.6123 | 404.40 | 0.0669 | 3.76801 | 8.92 |
| 24.1059 | 685.67 | 0.0836 | 3.69196 | 15.12 |
| 24.7860 | 214.25 | 0.2676 | 3.59216 | 4.72 |
| 25.1731 | 411.67 | 0.1338 | 3.53780 | 9.08 |
| 25.3327 | 594.33 | 0.1004 | 3.51587 | 13.10 |
| 25.6858 | 579.80 | 0.1506 | 3.46833 | 12.78 |
| 26.1302 | 158.57 | 0.2007 | 3.41036 | 3.50 |
| 26.8105 | 216.83 | 0.2007 | 3.32535 | 4.78 |
| 27.1097 | 433.11 | 0.0836 | 3.28932 | 9.55 |
| 27.8621 | 169.00 | 0.2007 | 3.20217 | 3.73 |
| 28.3625 | 239.97 | 0.1338 | 3.14681 | 5.29 |
| 28.7867 | 977.27 | 0.1004 | 3.10139 | 21.55 |
| 30.6844 | 114.31 | 0.2007 | 2.91378 | 2.52 |
| 31.2736 | 112.79 | 0.1673 | 2.86021 | 2.49 |
| 32.1023 | 38.97 | 0.2007 | 2.78825 | 0.86 |
| 33.4696 | 49.08 | 0.2676 | 2.67740 | 1.08 |
| 33.9991 | 93.88 | 0.1338 | 2.63690 | 2.07 |
| 36.8877 | 25.25 | 0.2007 | 2.43678 | 0.56 |
| 38.2121 | 65.18 | 0.2676 | 2.35532 | 1.44 |

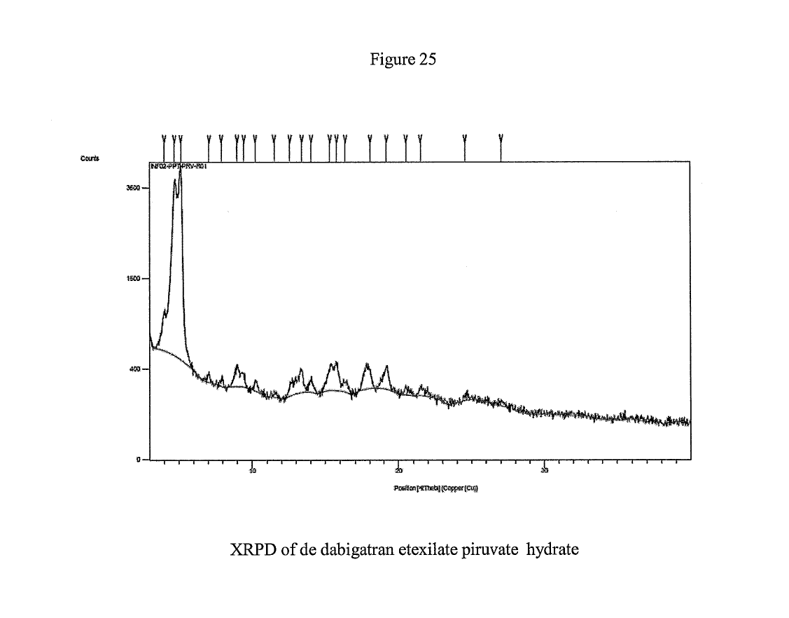

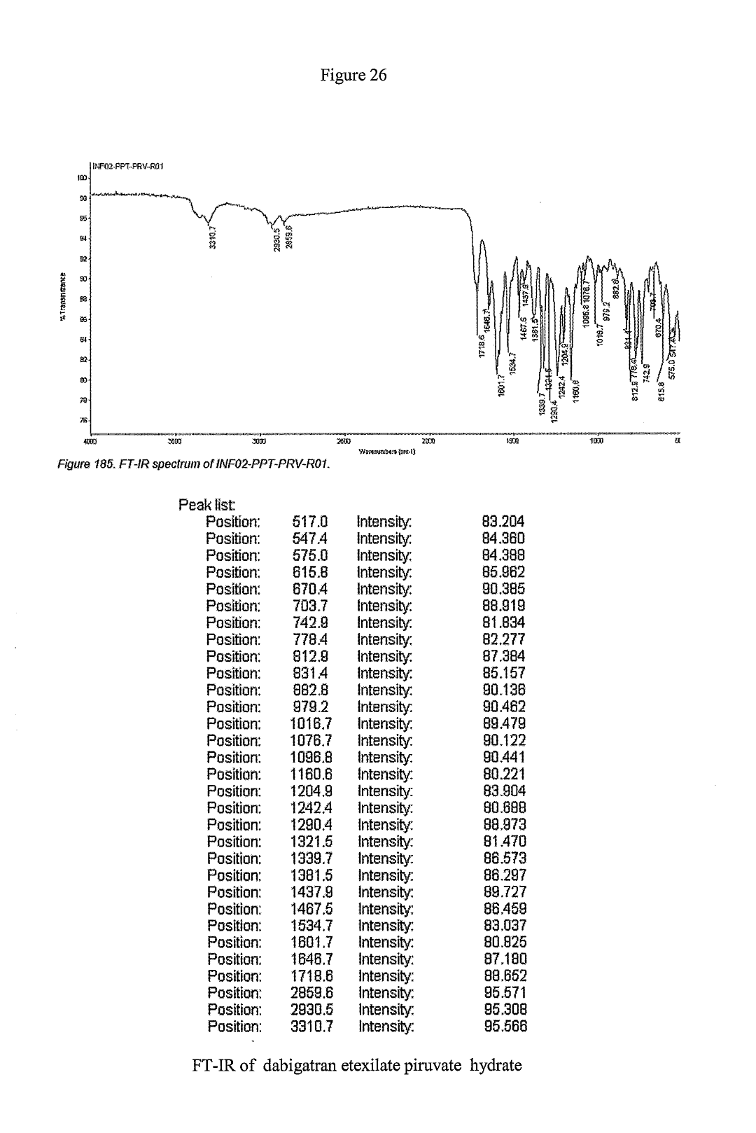

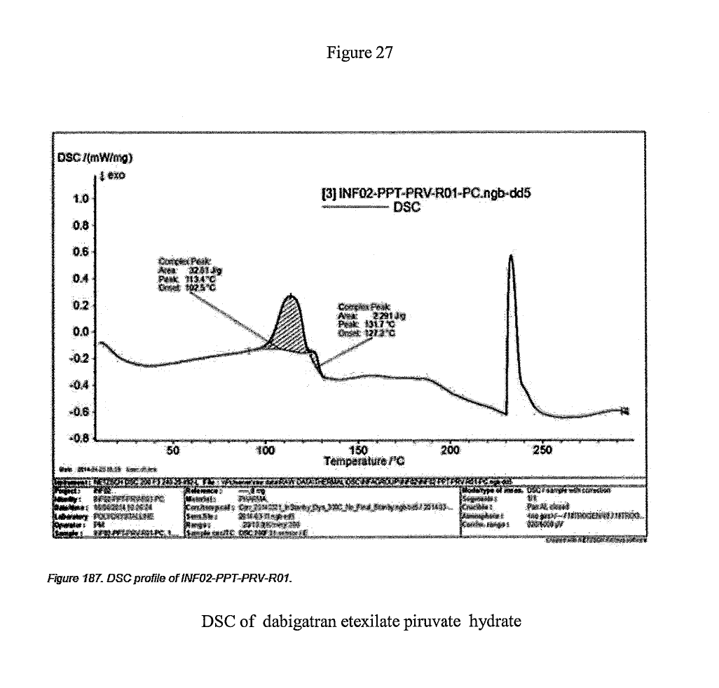

According to another preferred embodiment, the invention relates to dabigatran etexilate pyruvate hydrate showing the X-ray diffraction pattern of FIG. 25, the FT-IR spectrum of FIG. 26, the DSC profile of FIG. 27 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.0081 | 486.90 | 0.1338 | 22.04569 | 12.99 |
| 4.7132 | 3265.56 | 0.1004 | 18.74892 | 87.14 |
| 5.1331 | 3747.48 | 0.1840 | 17.21608 | 100.00 |
| 7.0593 | 61.56 | 0.1171 | 12.52226 | 1.64 |
| 7.9185 | 48.91 | 0.2007 | 11.16547 | 1.31 |
| 9.0008 | 160.40 | 0.3011 | 9.82509 | 4.28 |
| 9.4487 | 81.56 | 0.2342 | 9.36032 | 2.18 |
| 10.2235 | 74.07 | 0.2007 | 8.65264 | 1.98 |
| 11.5114 | 24.47 | 0.4015 | 7.68733 | 0.65 |
| 12.6126 | 86.12 | 0.2342 | 7.01847 | 2.30 |
| 13.4166 | 168.07 | 0.2007 | 6.59964 | 4.48 |
| 14.0523 | 94.42 | 0.2007 | 6.30252 | 2.52 |
| 15.3464 | 194.95 | 0.2007 | 5.77384 | 5.20 |
| 15.7826 | 222.79 | 0.1673 | 5.61523 | 5.95 |
| 16.3694 | 71.78 | 0.2676 | 5.41521 | 1.92 |
| 18.0767 | 135.57 | 0.2007 | 4.90743 | 3.62 |
| 19.2087 | 180.42 | 0.2007 | 4.62072 | 4.81 |
| 20.5116 | 51.75 | 0.2007 | 4.33006 | 1.38 |
| 21.5201 | 60.69 | 0.2007 | 4.12937 | 1.62 |
| 24.5712 | 24.25 | 0.3346 | 3.62308 | 0.65 |
| 27.0835 | 25.25 | 0.4015 | 3.29243 | 0.67 |

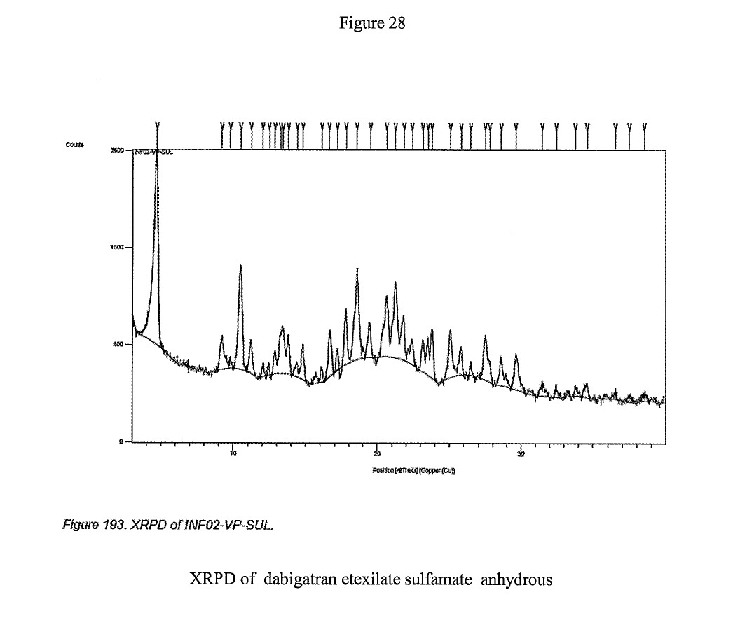

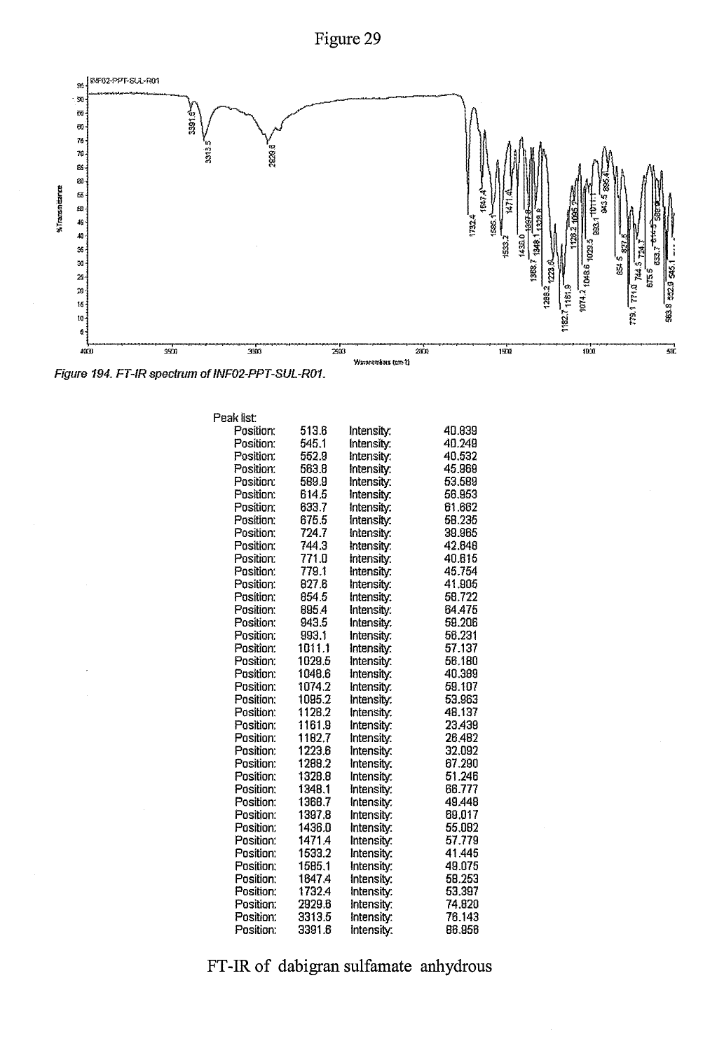

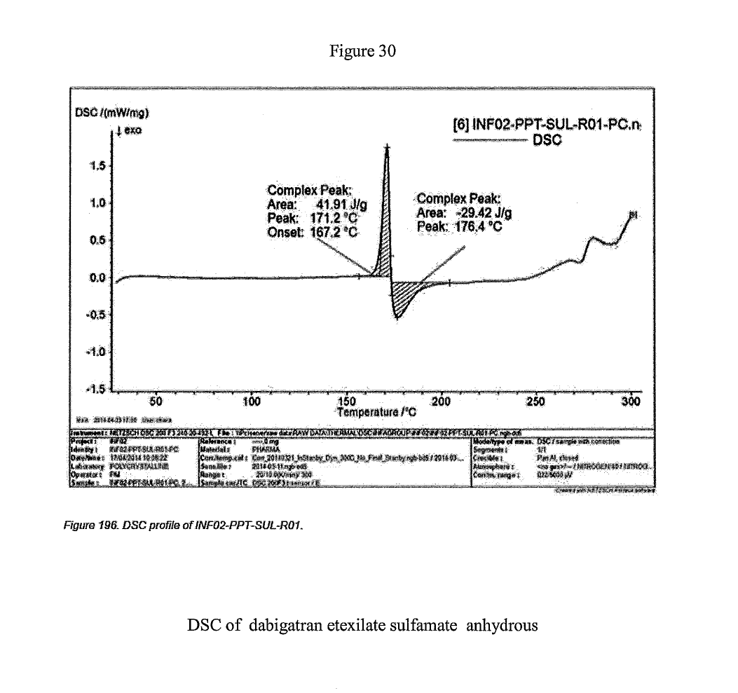

According to another preferred embodiment, the invention relates to anhydrous dabigatran etexilate sulfamate showing the X-ray diffraction pattern of FIG. 28, the FT-IR spectrum of FIG. 29, the DSC profile of FIG. 30 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.7063 | 2957.15 | 0.2175 | 18.77650 | 100.00 |
| 9.2070 | 221.81 | 0.2007 | 9.60550 | 7.50 |
| 9.7890 | 64.16 | 0.1338 | 9.03566 | 2.17 |
| 10.5058 | 1110.05 | 0.2007 | 8.42075 | 37.54 |
| 11.2031 | 248.05 | 0.1004 | 7.89816 | 8.39 |
| 12.0391 | 62.66 | 0.1004 | 7.35154 | 2.12 |
| 12.4627 | 71.13 | 0.0836 | 7.10260 | 2.41 |
| 12.8573 | 143.02 | 0.1338 | 6.88544 | 4.84 |
| 13.2387 | 279.44 | 0.1338 | 6.68796 | 9.45 |
| 13.4302 | 367.57 | 0.1673 | 6.59301 | 12.43 |
| 13.8175 | 270.63 | 0.1673 | 6.40906 | 9.15 |
| 14.4198 | 77.77 | 0.1338 | 6.14271 | 2.63 |
| 14.8067 | 241.60 | 0.1171 | 5.98304 | 8.17 |
| 16.1079 | 73.06 | 0.1338 | 5.50256 | 2.47 |
| 16.6399 | 305.60 | 0.0669 | 5.32780 | 10.33 |
| 17.1879 | 140.20 | 0.1673 | 5.15915 | 4.74 |
| 17.7887 | 466.85 | 0.1506 | 4.98625 | 15.79 |
| 18.5684 | 923.75 | 0.1004 | 4.77859 | 31.24 |
| 19.4945 | 276.81 | 0.1338 | 4.55362 | 9.36 |
| 20.6216 | 601.93 | 0.1171 | 4.30720 | 20.36 |
| 21.2288 | 788.73 | 0.1673 | 4.18536 | 26.67 |
| 21.8407 | 372.02 | 0.1004 | 4.06947 | 12.58 |
| 22.4146 | 185.46 | 0.1338 | 3.96655 | 6.27 |
| 23.1307 | 229.96 | 0.1004 | 3.84535 | 7.78 |
| 23.4961 | 275.25 | 0.0669 | 3.78637 | 9.31 |
| 23.7882 | 366.83 | 0.1004 | 3.74053 | 12.40 |
| 25.0434 | 362.01 | 0.1004 | 3.55582 | 12.24 |
| 25.8087 | 191.90 | 0.0669 | 3.45210 | 6.49 |
| 26.4646 | 50.79 | 0.1673 | 3.36801 | 1.72 |
| 27.4987 | 326.77 | 0.1338 | 3.24367 | 11.05 |
| 27.8064 | 125.41 | 0.1004 | 3.20846 | 4.24 |
| 28.5967 | 177.20 | 0.1338 | 3.12157 | 5.99 |
| 29.6135 | 189.59 | 0.1673 | 3.01666 | 6.41 |
| 31.4741 | 49.84 | 0.3346 | 2.84245 | 1.69 |
| 32.4424 | 40.64 | 0.1338 | 2.75979 | 1.37 |
| 33.7762 | 40.43 | 0.1673 | 2.65380 | 1.37 |
| 34.5849 | 60.41 | 0.1004 | 2.59357 | 2.04 |
| 36.5442 | 22.50 | 0.2676 | 2.45889 | 0.76 |
| 37.5209 | 22.62 | 0.3346 | 2.39710 | 0.76 |
| 38.5171 | 21.35 | 0.4015 | 2.33736 | 0.72 |

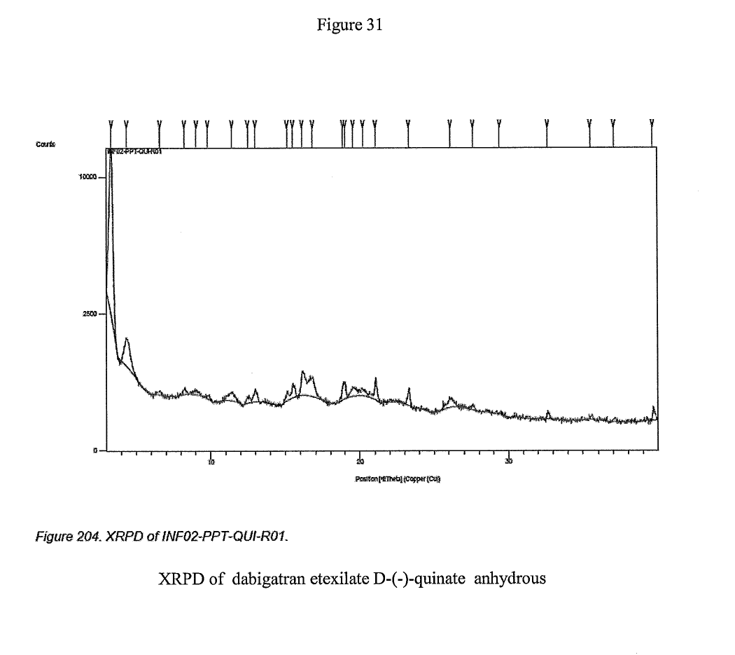

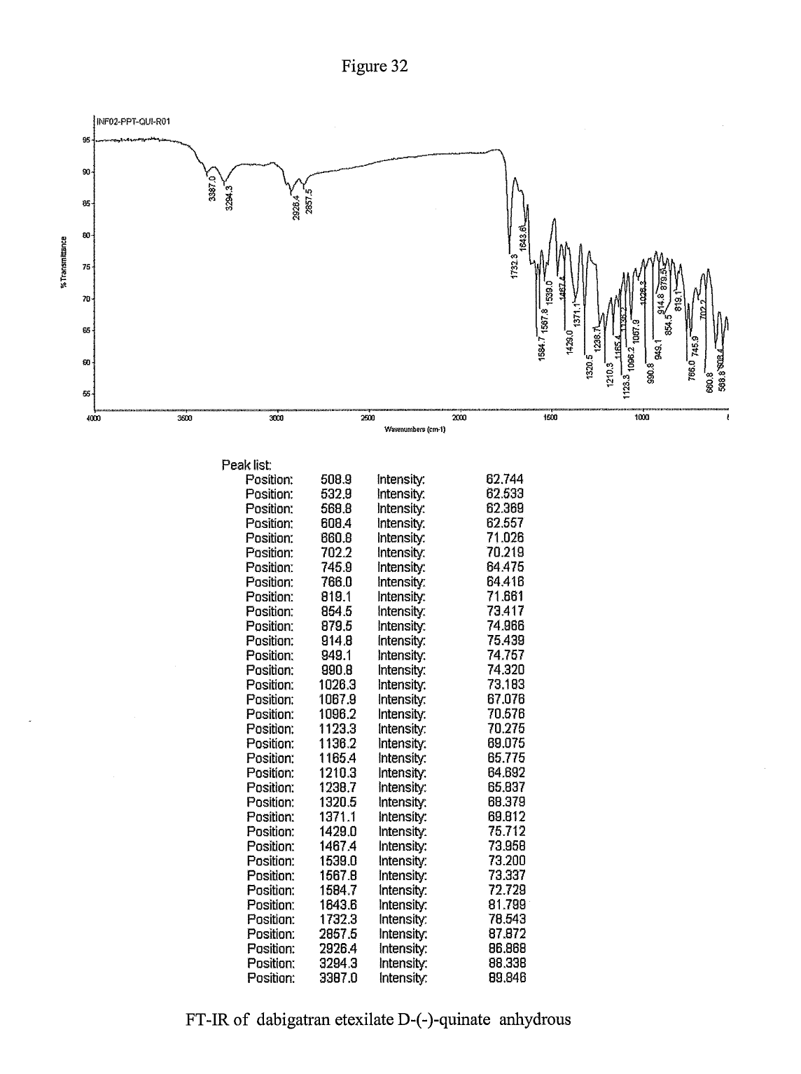

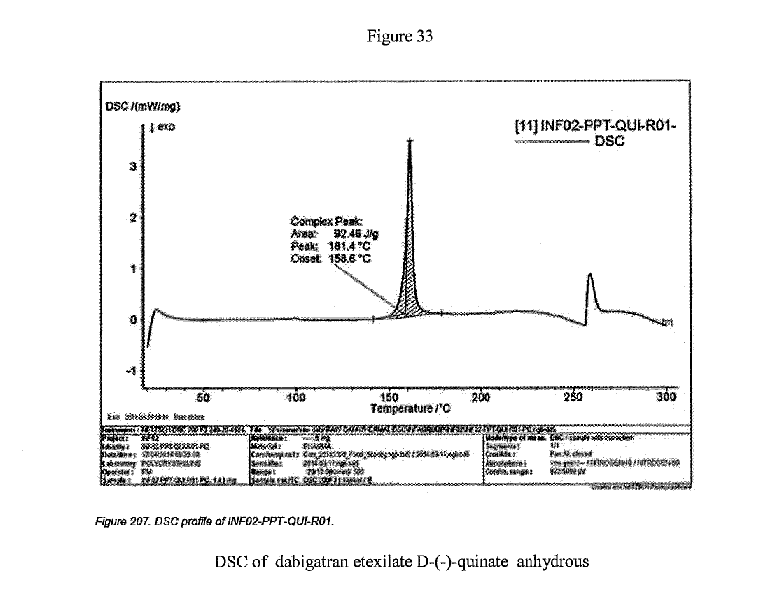

According to another preferred embodiment, the invention relates to anhydrous dabigatran etexilate D-(−)-quinate showing the X-ray diffraction pattern of FIG. 31, the FT-IR spectrum of FIG. 32, the DSC profile of FIG. 33 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 3.3667 | 9808.05 | 0.2007 | 26.24396 | 100.00 |
| 4.3670 | 757.67 | 0.3011 | 20.23460 | 7.72 |
| 6.5806 | 55.22 | 0.4015 | 13.43211 | 0.56 |
| 8.2317 | 88.88 | 0.2007 | 10.74129 | 0.91 |
| 9.0439 | 60.06 | 0.4015 | 9.77841 | 0.61 |
| 9.8218 | 56.84 | 0.2676 | 9.00562 | 0.58 |
| 11.4402 | 123.46 | 0.2007 | 7.73497 | 1.26 |
| 12.5173 | 75.13 | 0.2007 | 7.07170 | 0.77 |
| 13.0094 | 183.26 | 0.1673 | 6.80530 | 1.87 |
| 15.1401 | 124.93 | 0.1004 | 5.85204 | 1.27 |
| 15.5147 | 205.60 | 0.0669 | 5.71156 | 2.10 |
| 16.1169 | 403.23 | 0.1506 | 5.49950 | 4.11 |
| 16.8284 | 320.06 | 0.1338 | 5.26855 | 3.26 |
| 18.8733 | 250.39 | 0.1338 | 4.70207 | 2.55 |
| 19.0128 | 264.20 | 0.0669 | 4.66788 | 2.69 |
| 19.5538 | 132.00 | 0.2342 | 4.53994 | 1.35 |
| 20.2053 | 94.06 | 0.3346 | 4.39501 | 0.96 |
| 21.0493 | 381.34 | 0.0669 | 4.22064 | 3.89 |
| 23.2960 | 255.73 | 0.1171 | 3.81844 | 2.61 |
| 26.1298 | 105.39 | 0.3346 | 3.41040 | 1.07 |
| 27.6471 | 49.35 | 0.1673 | 3.22659 | 0.50 |
| 29.4563 | 17.74 | 0.4015 | 3.03241 | 0.18 |
| 32.6878 | 53.63 | 0.1338 | 2.73963 | 0.55 |
| 35.5378 | 24.31 | 0.2007 | 2.52618 | 0.25 |
| 37.1100 | 23.51 | 0.2007 | 2.42270 | 0.24 |
| 39.7022 | 130.29 | 0.0836 | 2.27029 | 1.33 |

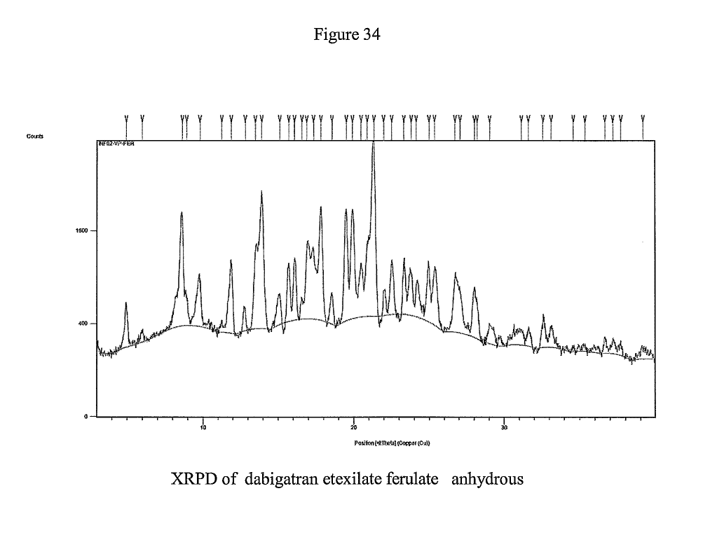

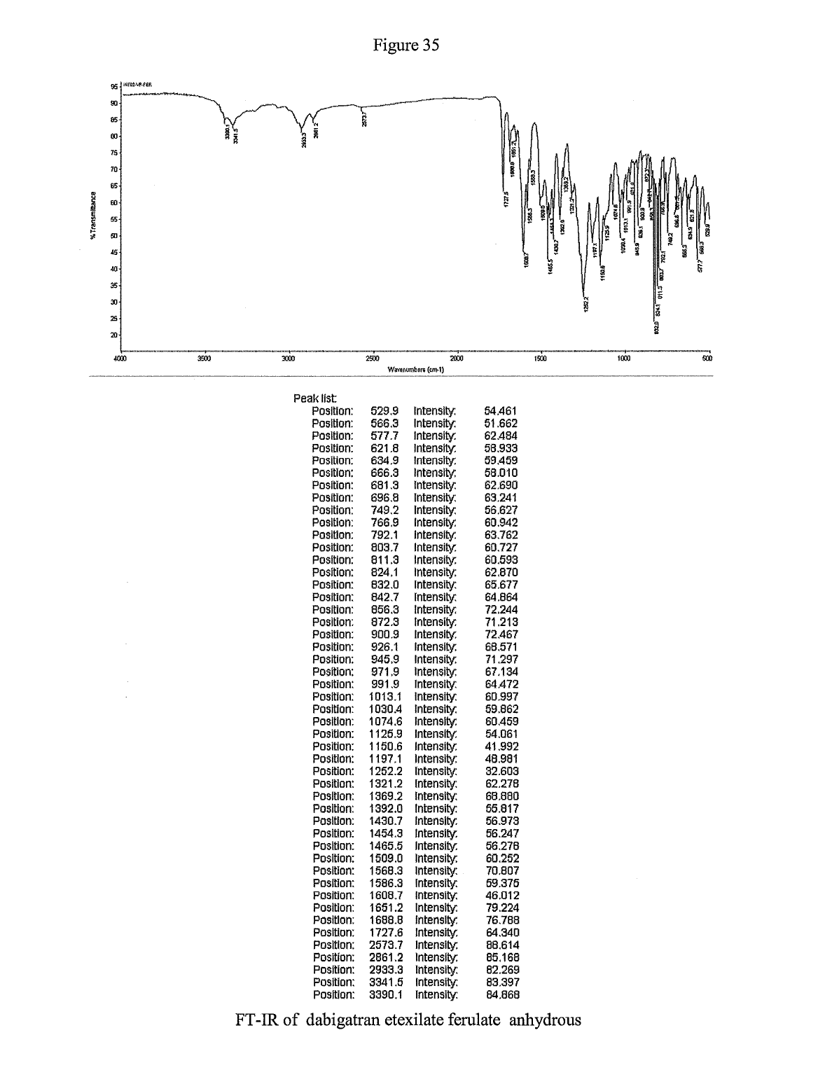

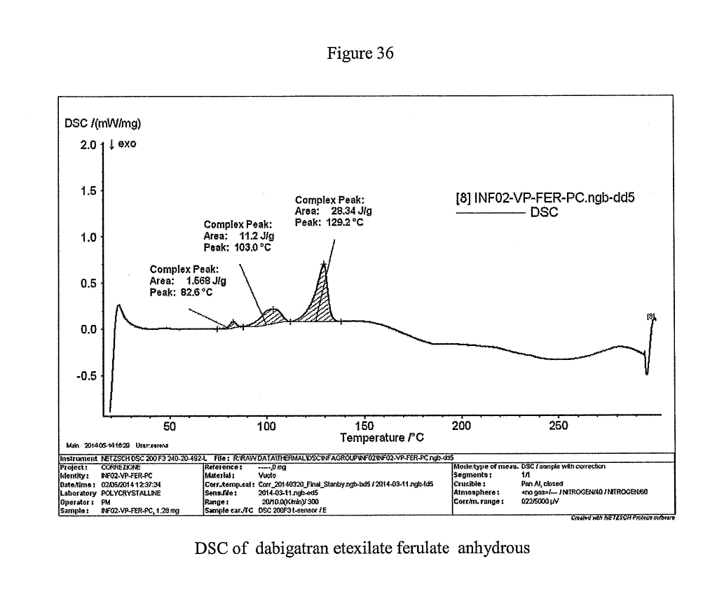

According to another preferred embodiment, the invention relates to anhydrous dabigatran etexilate ferulate showing the X-ray diffraction pattern of FIG. 34, the FT-IR spectrum of FIG. 35, the DSC profile of FIG. 36 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.9329 | 387.74 | 0.0836 | 17.91430 | 12.69 |
| 5.9904 | 74.95 | 0.1673 | 14.75409 | 2.45 |
| 8.6395 | 1460.10 | 0.1673 | 10.23515 | 47.78 |
| 8.9097 | 291.99 | 0.1004 | 9.92536 | 9.56 |
| 9.7773 | 533.53 | 0.1673 | 9.04650 | 17.46 |
| 11.2414 | 93.26 | 0.1673 | 7.87134 | 3.05 |
| 11.8889 | 760.20 | 0.1673 | 7.44404 | 24.88 |
| 12.8069 | 201.01 | 0.1004 | 6.91244 | 6.58 |
| 13.4916 | 929.06 | 0.1338 | 6.56316 | 30.40 |
| 13.9104 | 1999.65 | 0.1506 | 6.36645 | 65.44 |
| 15.1067 | 297.69 | 0.2342 | 5.86489 | 9.74 |
| 15.7094 | 671.68 | 0.1840 | 5.64121 | 21.98 |
| 16.0783 | 697.52 | 0.1506 | 5.51262 | 22.83 |
| 16.5740 | 179.50 | 0.1338 | 5.34884 | 5.87 |
| 16.8904 | 863.22 | 0.1171 | 5.24935 | 28.25 |
| 17.3224 | 861.62 | 0.0836 | 5.11941 | 28.20 |
| 17.8213 | 1618.18 | 0.2007 | 4.97720 | 52.95 |
| 18.5616 | 297.69 | 0.1673 | 4.78031 | 9.74 |
| 19.5157 | 1558.99 | 0.1840 | 4.54871 | 51.02 |
| 19.9169 | 1544.83 | 0.2175 | 4.45799 | 50.55 |
| 20.4936 | 625.48 | 0.1171 | 4.33382 | 20.47 |
| 20.8817 | 908.83 | 0.0836 | 4.25415 | 29.74 |
| 21.3390 | 3055.78 | 0.1673 | 4.16399 | 100.00 |
| 21.9828 | 262.95 | 0.0669 | 4.04349 | 8.61 |
| 22.5119 | 661.09 | 0.1506 | 3.94962 | 21.63 |
| 23.3423 | 649.91 | 0.1673 | 3.81096 | 21.27 |
| 23.8209 | 485.00 | 0.2342 | 3.73548 | 15.87 |
| 24.1601 | 344.10 | 0.1338 | 3.68379 | 11.26 |
| 25.0031 | 622.77 | 0.1673 | 3.56147 | 20.38 |
| 25.3961 | 636.44 | 0.0836 | 3.50724 | 20.83 |
| 26.7489 | 593.83 | 0.2342 | 3.33286 | 19.43 |
| 27.0713 | 407.44 | 0.2007 | 3.29390 | 13.33 |
| 28.0148 | 488.71 | 0.1171 | 3.18507 | 15.99 |
| 28.1939 | 291.32 | 0.1673 | 3.16524 | 9.53 |
| 29.0287 | 143.40 | 0.4015 | 3.07609 | 4.69 |
| 31.1498 | 107.11 | 0.3346 | 2.87130 | 3.51 |
| 31.6269 | 129.25 | 0.1673 | 2.82906 | 4.23 |
| 32.6074 | 265.91 | 0.0669 | 2.74620 | 8.70 |
| 33.1258 | 147.43 | 0.2007 | 2.70440 | 4.82 |
| 34.6037 | 32.23 | 0.2007 | 2.59221 | 1.05 |
| 35.3813 | 38.03 | 0.2342 | 2.53700 | 1.24 |
| 36.6945 | 94.46 | 0.1673 | 2.44916 | 3.09 |
| 37.2141 | 94.51 | 0.1338 | 2.41615 | 3.09 |
| 37.7469 | 53.73 | 0.1673 | 2.38327 | 1.76 |
| 39.1771 | 61.73 | 0.3346 | 2.29950 | 2.02 |

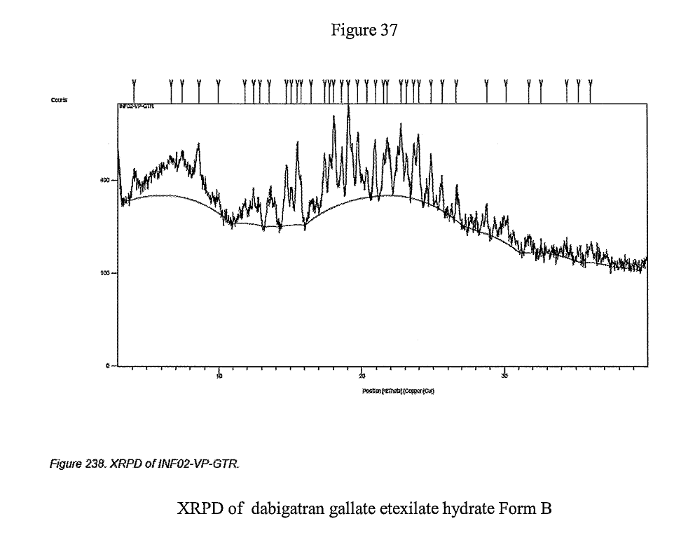

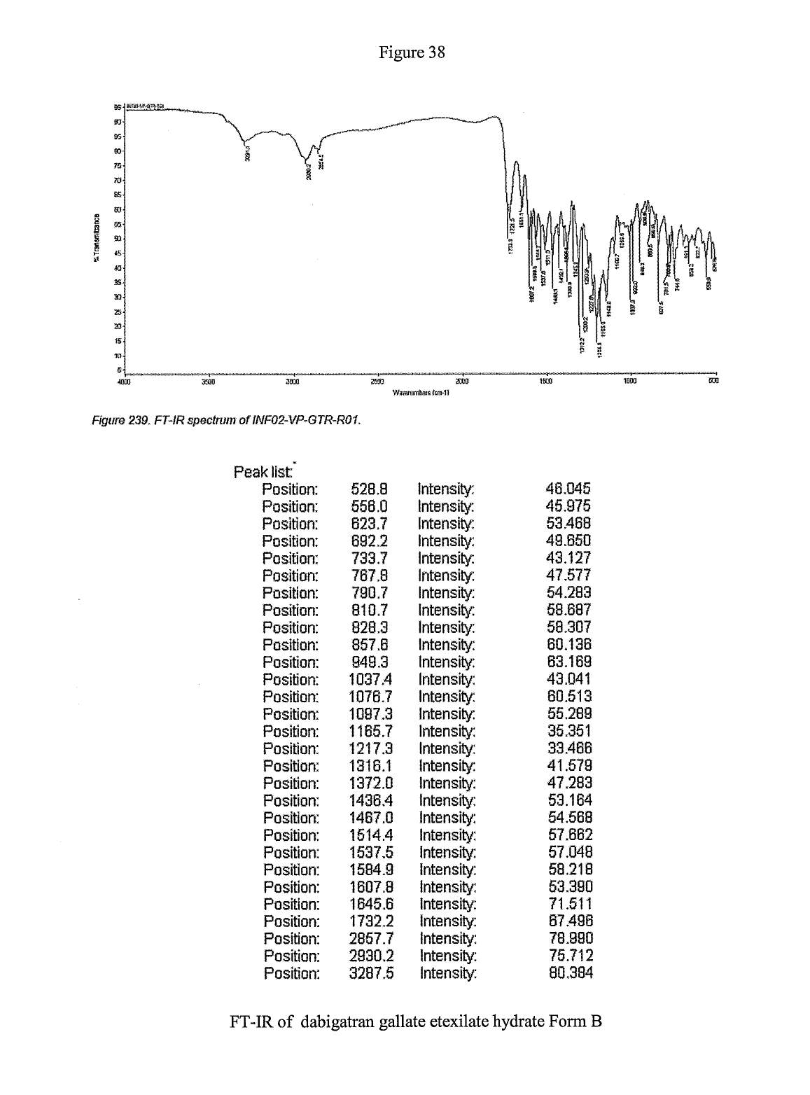

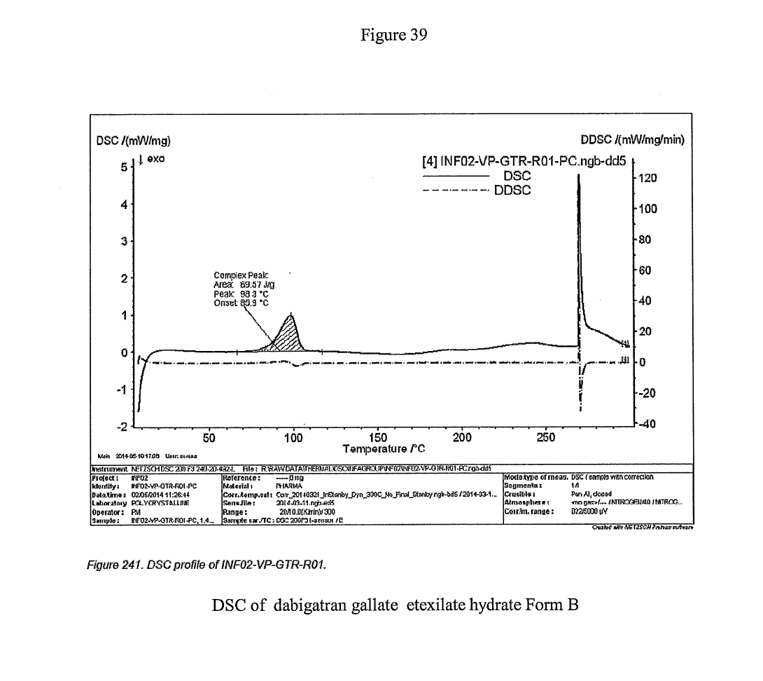

According to another preferred embodiment, the invention relates to dabigatran etexilate gallate hydrate Form B (ratio dabigatran/gallate 1/2) showing the X-ray diffraction pattern of FIG. 37, the FT-IR spectrum of FIG. 38, the DSC profile of FIG. 39 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 6.8617 | 185.59 | 0.1673 | 12.88250 | 72.12 |
| 10.4424 | 257.32 | 0.1338 | 8.47172 | 100.00 |
| 13.2774 | 76.22 | 0.2007 | 6.66854 | 29.62 |
| 14.1407 | 145.14 | 0.4015 | 6.26330 | 56.40 |
| 16.3580 | 25.94 | 0.4015 | 5.41898 | 10.08 |
| 17.6399 | 21.69 | 0.9368 | 5.02797 | 8.43 |
| 24.7258 | 32.64 | 0.3346 | 3.60077 | 12.68 |
| 25.3863 | 124.24 | 0.1004 | 3.50857 | 48.28 |
| 26.4163 | 82.51 | 0.1673 | 3.37406 | 32.06 |
| 27.6649 | 35.97 | 0.2676 | 3.22455 | 13.98 |

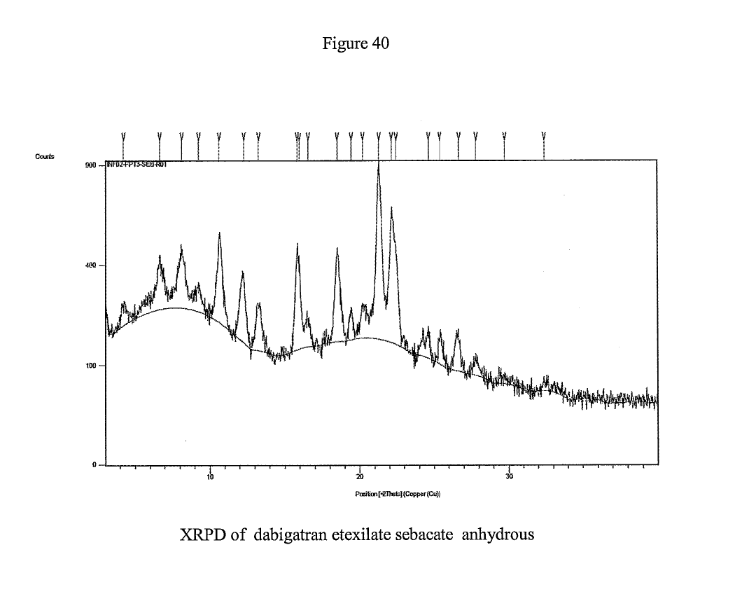

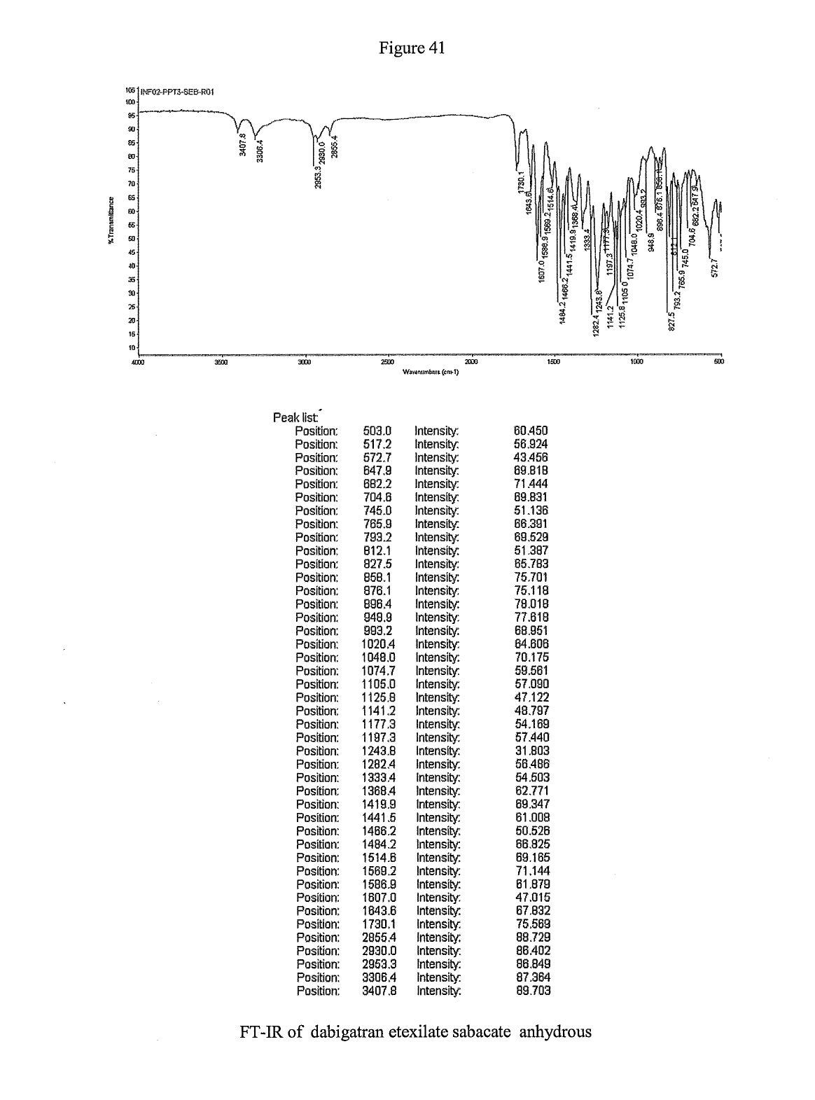

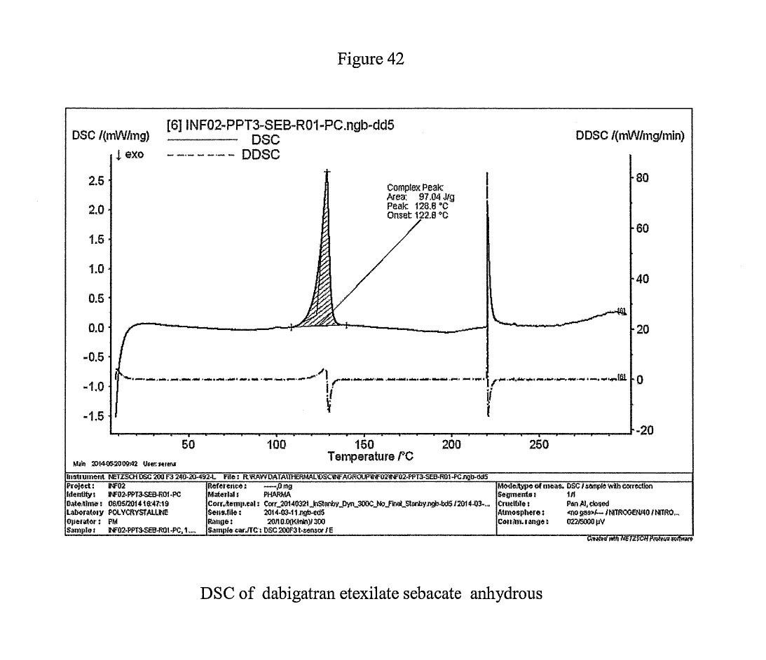

According to another preferred embodiment, the invention relates to anhydrous dabigatran etexilate sebacate showing the X-ray diffraction pattern of FIG. 40, the FT-IR spectrum of FIG. 41, the DSC profile of FIG. 42 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.2236 | 54.70 | 0.3346 | 20.92105 | 7.07 |
| 6.6588 | 192.74 | 0.2007 | 13.27463 | 24.92 |
| 8.1342 | 208.74 | 0.2007 | 10.86984 | 26.99 |
| 9.2622 | 85.79 | 0.2676 | 9.54837 | 11.09 |
| 10.6376 | 314.17 | 0.1004 | 8.31673 | 40.62 |
| 12.2754 | 196.00 | 0.3011 | 7.21053 | 25.34 |
| 13.2778 | 127.08 | 0.3346 | 6.66835 | 16.43 |
| 15.8470 | 341.27 | 0.0836 | 5.59256 | 44.13 |
| 16.0051 | 254.39 | 0.1004 | 5.53765 | 32.89 |
| 16.5680 | 71.64 | 0.2676 | 5.35077 | 9.26 |
| 18.5242 | 303.30 | 0.2007 | 4.78990 | 39.22 |
| 19.4438 | 80.32 | 0.2342 | 4.56537 | 10.39 |
| 20.2156 | 95.11 | 0.3346 | 4.39279 | 12.30 |
| 21.3023 | 773.40 | 0.1171 | 4.17108 | 100.00 |
| 22.1502 | 488.15 | 0.1673 | 4.01330 | 63.12 |
| 22.4667 | 294.00 | 0.1673 | 3.95747 | 38.01 |
| 24.6341 | 64.43 | 0.2007 | 3.61397 | 8.33 |
| 25.4078 | 60.30 | 0.2676 | 3.50566 | 7.80 |
| 26.7016 | 63.75 | 0.3011 | 3.33865 | 8.24 |
| 27.8367 | 31.21 | 0.2676 | 3.20504 | 4.04 |
| 29.7420 | 10.00 | 0.8029 | 3.00392 | 1.29 |
| 32.4348 | 13.09 | 0.4015 | 2.76042 | 1.69 |

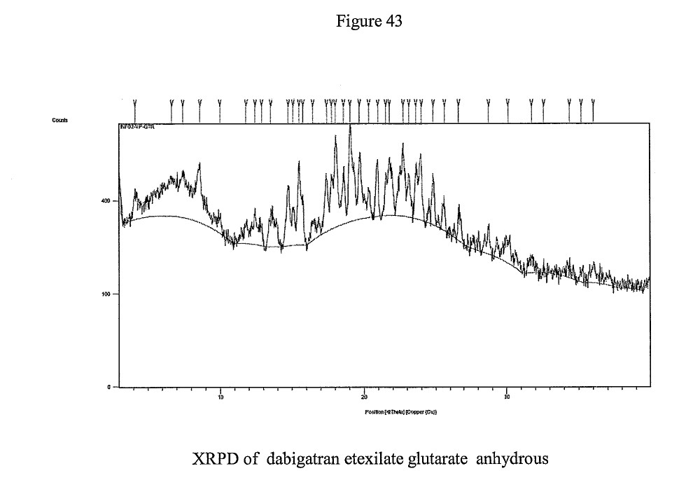

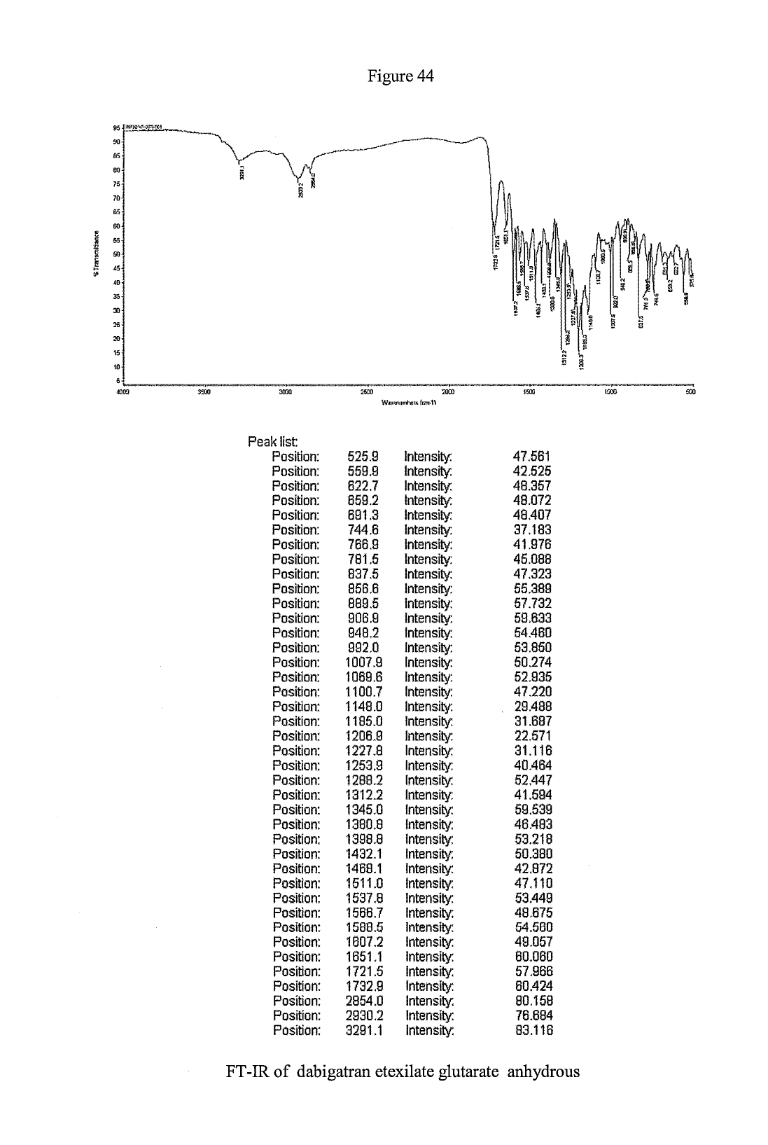

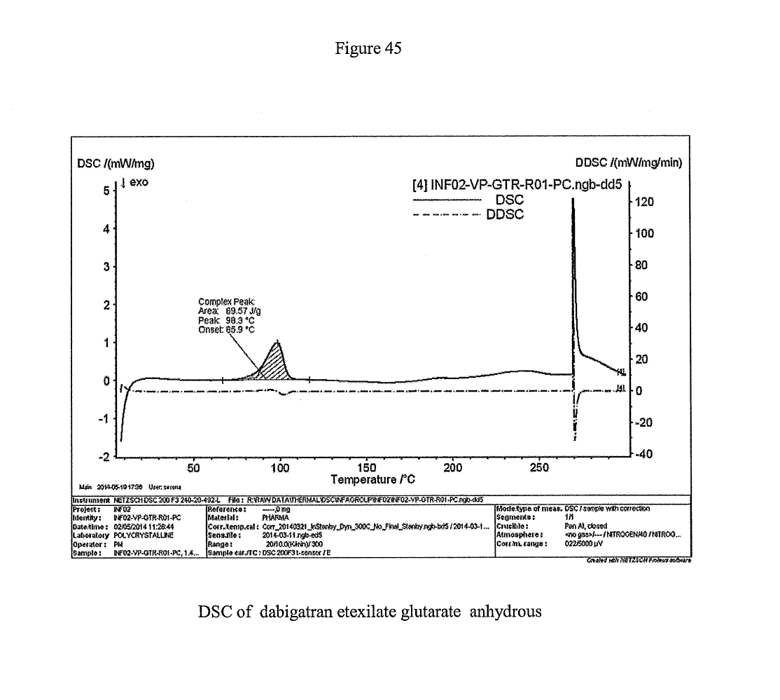

According to another preferred embodiment, the invention relates to anhydrous dabigatran etexilate glutarate showing the X-ray diffraction pattern of FIG. 43, the FT-IR spectrum of FIG. 44, the DSC profile of FIG. 45 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.1215 | 105.31 | 0.2007 | 21.43951 | 21.54 |
| 6.6703 | 162.11 | 0.4015 | 13.25165 | 33.16 |
| 7.4386 | 195.17 | 0.2007 | 11.88462 | 39.92 |
| 8.6000 | 253.01 | 0.2342 | 10.28202 | 51.75 |
| 9.9574 | 60.83 | 0.2007 | 8.88322 | 12.44 |
| 11.8067 | 73.53 | 0.2007 | 7.49567 | 15.04 |
| 12.4282 | 137.54 | 0.1004 | 7.12219 | 28.13 |
| 12.8757 | 68.75 | 0.2676 | 6.87565 | 14.06 |
| 13.5268 | 107.23 | 0.3346 | 6.54617 | 21.93 |
| 14.7442 | 233.69 | 0.1673 | 6.00826 | 47.80 |
| 15.0860 | 130.72 | 0.1338 | 5.87289 | 26.74 |
| 15.5039 | 333.55 | 0.0836 | 5.71555 | 68.22 |
| 15.7778 | 154.57 | 0.1004 | 5.61693 | 31.62 |
| 16.4662 | 61.58 | 0.2007 | 5.38362 | 12.60 |
| 17.4204 | 224.95 | 0.1673 | 5.09083 | 46.01 |
| 17.7474 | 212.58 | 0.1004 | 4.99774 | 43.48 |
| 18.0320 | 391.17 | 0.0836 | 4.91950 | 80.01 |
| 18.6097 | 212.41 | 0.1673 | 4.76808 | 43.45 |
| 19.0538 | 488.89 | 0.1004 | 4.65794 | 100.00 |
| 19.6893 | 280.41 | 0.1004 | 4.50899 | 57.36 |
| 20.3462 | 111.50 | 0.1338 | 4.36488 | 22.81 |
| 20.9498 | 253.02 | 0.0836 | 4.24047 | 51.75 |
| 21.5078 | 172.94 | 0.1004 | 4.13170 | 35.37 |
| 21.7500 | 247.38 | 0.1004 | 4.08623 | 50.60 |
| 22.7288 | 315.20 | 0.1338 | 3.91242 | 64.47 |
| 23.1474 | 191.51 | 0.1338 | 3.84262 | 39.17 |
| 23.6366 | 222.51 | 0.1338 | 3.76418 | 45.51 |
| 24.0209 | 238.70 | 0.1004 | 3.70483 | 48.82 |
| 24.8526 | 202.63 | 0.1673 | 3.58270 | 41.45 |
| 25.6481 | 103.89 | 0.2007 | 3.47335 | 21.25 |
| 26.6562 | 112.62 | 0.1673 | 3.34424 | 23.04 |
| 28.7702 | 100.36 | 0.1171 | 3.10313 | 20.53 |
| 30.1338 | 66.08 | 0.4015 | 2.96575 | 13.52 |
| 31.7524 | 42.01 | 0.3346 | 2.81816 | 8.59 |
| 32.5936 | 9.22 | 0.2007 | 2.74733 | 1.89 |
| 34.4040 | 20.97 | 0.5353 | 2.60679 | 4.29 |
| 35.2249 | 25.15 | 0.2676 | 2.54790 | 5.14 |
| 36.0670 | 46.09 | 0.2007 | 2.49032 | 9.43 |

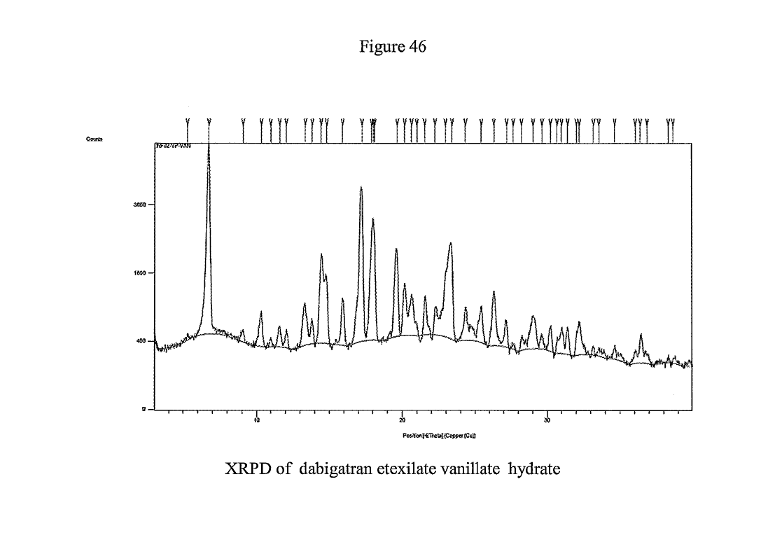

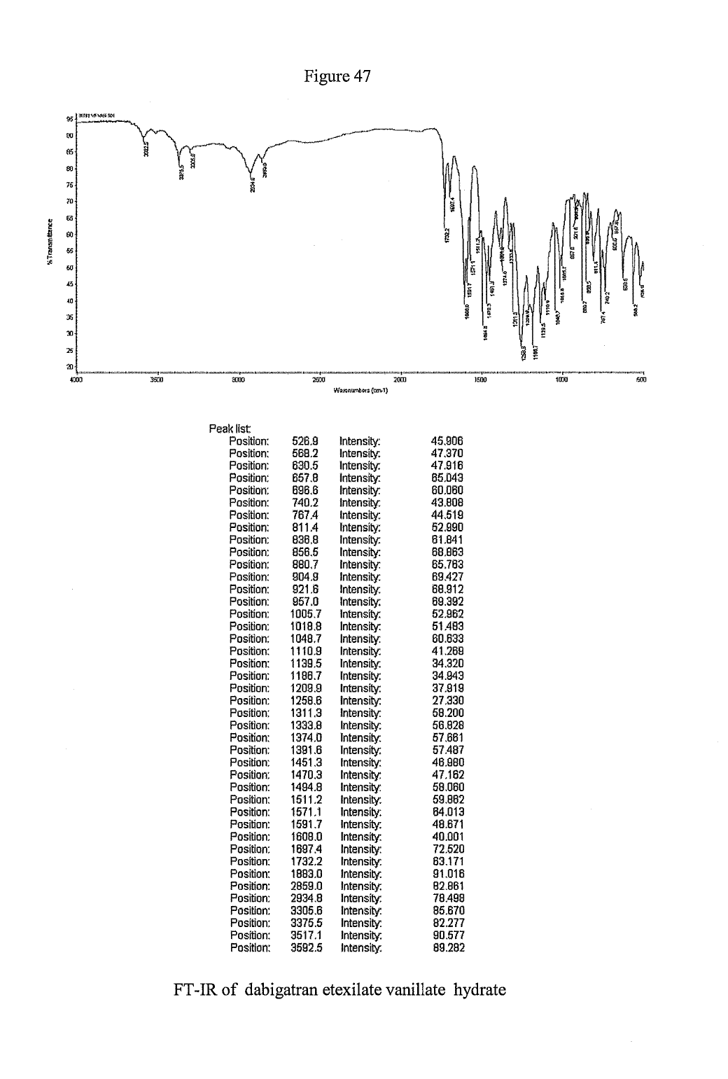

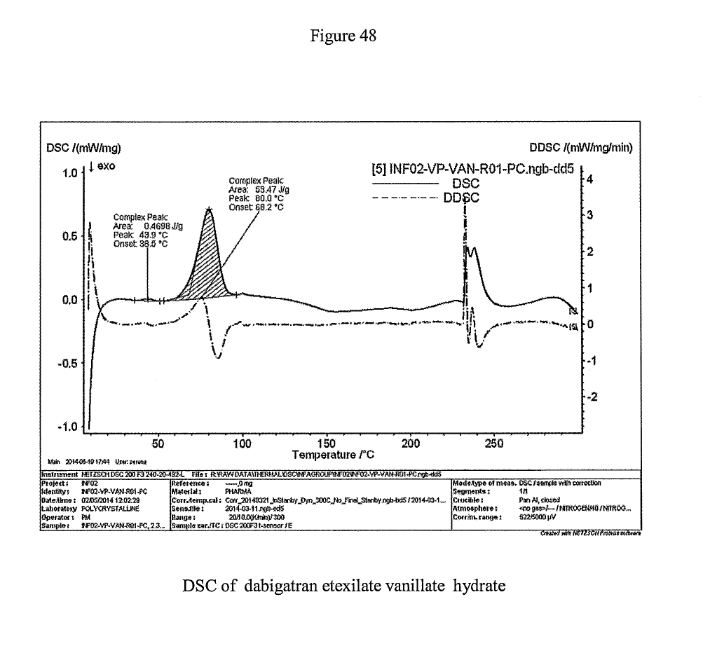

According to another preferred embodiment, the invention relates to dabigatran etexilate vanillate hydrate showing the X-ray diffraction pattern of FIG. 46, the FT-IR spectrum of FIG. 47, the DSC profile of FIG. 48 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 5.2816 | 50.74 | 0.2007 | 16.73234 | 0.94 |
| 6.7436 | 5423.85 | 0.2007 | 13.10776 | 100.00 |
| 9.0857 | 140.12 | 0.1673 | 9.73347 | 2.58 |
| 10.3175 | 489.93 | 0.1171 | 8.57400 | 9.03 |
| 10.9731 | 89.18 | 0.1004 | 8.06316 | 1.64 |
| 11.5976 | 253.82 | 0.1171 | 7.63033 | 4.68 |
| 12.0367 | 182.43 | 0.1004 | 7.35300 | 3.36 |
| 13.3462 | 578.66 | 0.0669 | 6.63434 | 10.67 |
| 13.8232 | 290.46 | 0.0669 | 6.40642 | 5.36 |
| 14.4398 | 1639.75 | 0.2342 | 6.13425 | 30.23 |
| 14.8237 | 1048.52 | 0.1338 | 5.97624 | 19.33 |
| 15.9067 | 718.91 | 0.1506 | 5.57170 | 13.25 |
| 17.2479 | 3501.87 | 0.2509 | 5.14135 | 64.56 |
| 17.9178 | 2306.53 | 0.0836 | 4.95061 | 42.53 |
| 18.0139 | 2724.40 | 0.1020 | 4.92034 | 50.23 |
| 18.1007 | 2157.84 | 0.0836 | 4.90099 | 39.78 |
| 19.6440 | 1607.43 | 0.2509 | 4.51929 | 29.64 |
| 20.1559 | 902.38 | 0.1673 | 4.40566 | 16.64 |
| 20.6228 | 671.84 | 0.1004 | 4.30696 | 12.39 |
| 20.9891 | 159.07 | 0.1338 | 4.23262 | 2.93 |
| 21.5416 | 634.36 | 0.1171 | 4.12530 | 11.70 |
| 22.2525 | 416.50 | 0.0836 | 3.99509 | 7.68 |
| 22.9683 | 1107.18 | 0.1004 | 3.87217 | 20.41 |
| 23.4004 | 1691.32 | 0.2175 | 3.80164 | 31.18 |
| 24.3324 | 479.38 | 0.0836 | 3.65809 | 8.84 |
| 25.4472 | 537.65 | 0.1338 | 3.50031 | 9.91 |
| 26.3185 | 861.57 | 0.1171 | 3.38637 | 15.88 |
| 27.2027 | 339.45 | 0.0669 | 3.27828 | 6.26 |
| 27.6486 | 51.94 | 0.2007 | 3.22641 | 0.96 |
| 28.2403 | 160.25 | 0.1338 | 3.16015 | 2.95 |
| 29.0257 | 425.95 | 0.3346 | 3.07640 | 7.85 |
| 29.6525 | 150.84 | 0.1673 | 3.01279 | 2.78 |
| 30.2465 | 319.55 | 0.1004 | 2.95495 | 5.89 |
| 30.6759 | 146.84 | 0.1338 | 2.91457 | 2.71 |
| 30.9967 | 306.32 | 0.1673 | 2.88513 | 5.65 |
| 31.4162 | 312.31 | 0.1171 | 2.84756 | 5.76 |
| 32.0217 | 274.83 | 0.1004 | 2.79508 | 5.07 |
| 32.2239 | 388.44 | 0.1673 | 2.77800 | 7.16 |
| 33.2056 | 71.32 | 0.1840 | 2.69808 | 1.32 |
| 33.5616 | 65.47 | 0.2007 | 2.67027 | 1.21 |
| 34.6642 | 125.37 | 0.1338 | 2.58782 | 2.31 |
| 36.0577 | 106.84 | 0.1673 | 2.49095 | 1.97 |
| 36.4106 | 287.17 | 0.0836 | 2.46761 | 5.29 |
| 36.8854 | 80.47 | 0.2007 | 2.43693 | 1.48 |
| 38.3514 | 42.58 | 0.1004 | 2.34708 | 0.79 |
| 38.6773 | 43.98 | 0.2007 | 2.32805 | 0.81 |

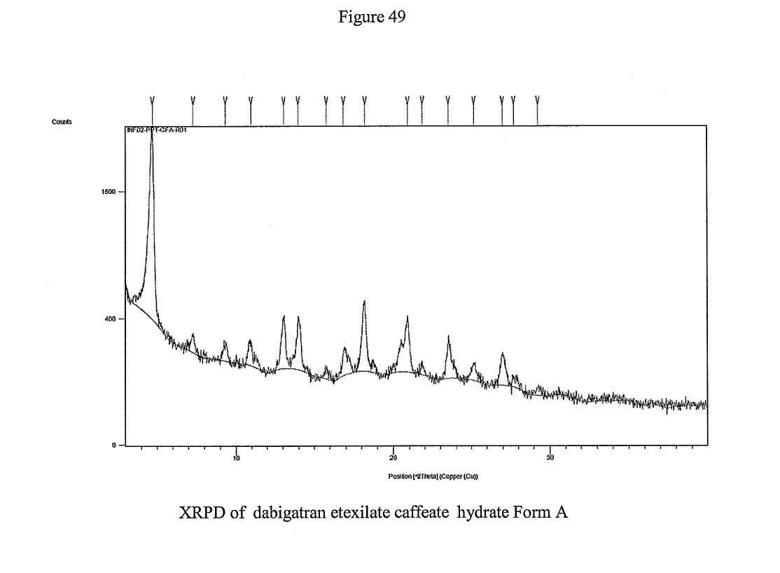

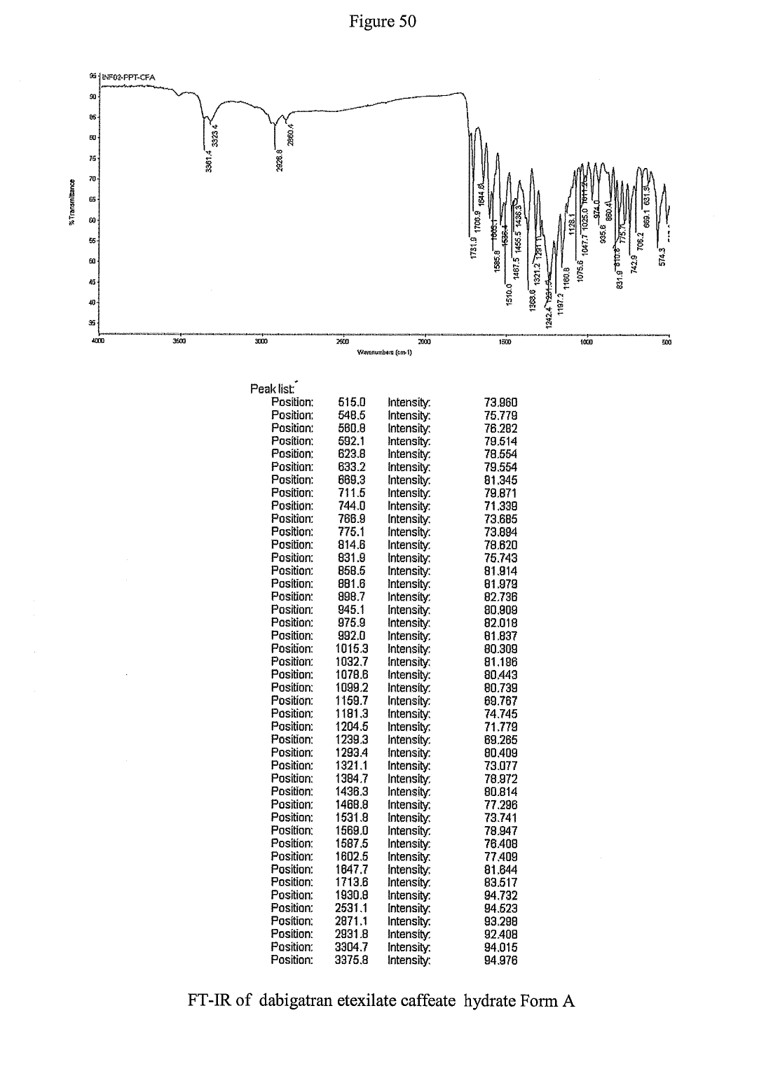

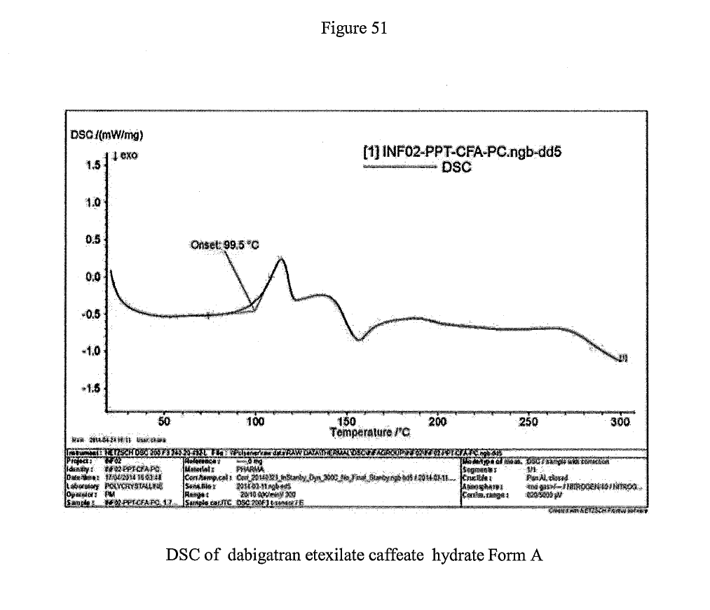

According to another preferred embodiment, the invention relates to dabigatran etexilate caffeate hydrate, form A, showing the X-ray diffraction pattern of FIG. 49, the FT-IR spectrum of FIG. 50, the DSC profile of FIG. 51 and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.6953 | 2099.82 | 0.1171 | 18.82044 | 100.00 |
| 7.2873 | 90.28 | 0.1673 | 12.13105 | 4.30 |
| 9.3340 | 73.95 | 0.2676 | 9.47509 | 3.52 |
| 10.9596 | 106.44 | 0.2007 | 8.07308 | 5.07 |
| 13.0804 | 255.55 | 0.2342 | 6.76854 | 12.17 |
| 13.9968 | 259.82 | 0.2342 | 6.32736 | 12.37 |
| 15.7906 | 30.56 | 0.3346 | 5.61239 | 1.46 |
| 16.8777 | 101.23 | 0.1673 | 5.25328 | 4.82 |
| 18.2239 | 338.07 | 0.1840 | 4.86813 | 16.10 |
| 20.9374 | 254.90 | 0.0669 | 4.24296 | 12.14 |
| 21.8646 | 37.52 | 0.2676 | 4.06507 | 1.79 |
| 23.5153 | 187.93 | 0.0669 | 3.78333 | 8.95 |
| 25.1838 | 61.12 | 0.1673 | 3.53632 | 2.91 |
| 26.9983 | 119.09 | 0.3011 | 3.30264 | 5.67 |
| 27.7483 | 27.92 | 0.4015 | 3.21505 | 1.33 |
| 29.2657 | 22.12 | 0.4015 | 3.05171 | 1.05 |

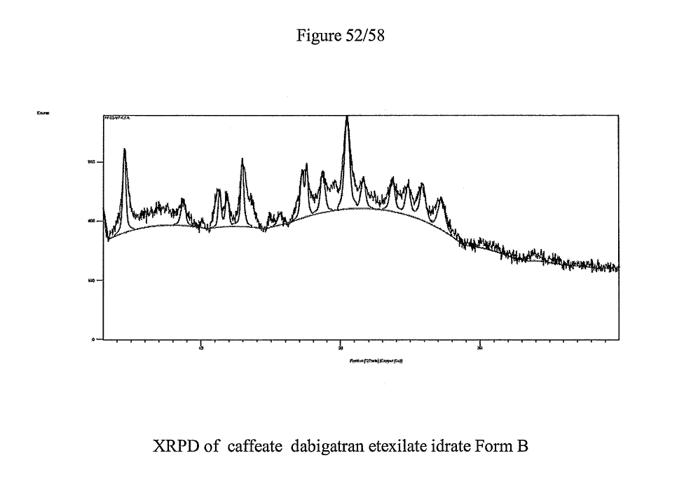

According to another preferred embodiment, the invention relates to dabigatran etexilate caffeate, Form B, obtained by vapour digestion, showing the X-ray diffraction pattern of FIG. 52, and the following characteristics of X-ray diffraction:

| Pos. | Height | FWHM | d-spacing | Rel. Int. |
|---|---|---|---|---|
| [°2Th.] | [cts] | [°2Th.] | [Å] | [%] |
| 4.5089 | 640.36 | 0.2342 | 19.59794 | 74.43 |
| 8.7131 | 173.20 | 0.3346 | 10.14881 | 20.13 |
| 11.3042 | 249.77 | 0.2007 | 7.82777 | 29.03 |
| 11.8302 | 215.77 | 0.2676 | 7.48085 | 25.08 |
| 12.9279 | 518.68 | 0.2342 | 6.84802 | 60.29 |
| 15.6350 | 73.19 | 0.4015 | 5.66789 | 8.51 |
| 17.1947 | 348.64 | 0.2676 | 5.15712 | 40.52 |
| 17.5288 | 391.63 | 0.2007 | 5.05957 | 45.52 |
| 18.6773 | 316.33 | 0.3346 | 4.75097 | 36.77 |
| 20.4486 | 860.33 | 0.3680 | 4.34326 | 100.00 |
| 21.5978 | 233.91 | 0.3346 | 4.11469 | 27.19 |
| 23.6989 | 243.31 | 0.4015 | 3.75442 | 28.28 |
| 24.8229 | 226.16 | 0.4015 | 3.58691 | 26.29 |
| 25.8741 | 262.39 | 0.4684 | 3.44352 | 30.50 |
| 27.2284 | 205.53 | 0.5353 | 3.27525 | 23.89 |
| 33.9602 | 30.56 | 0.8029 | 2.63983 | 3.55 |

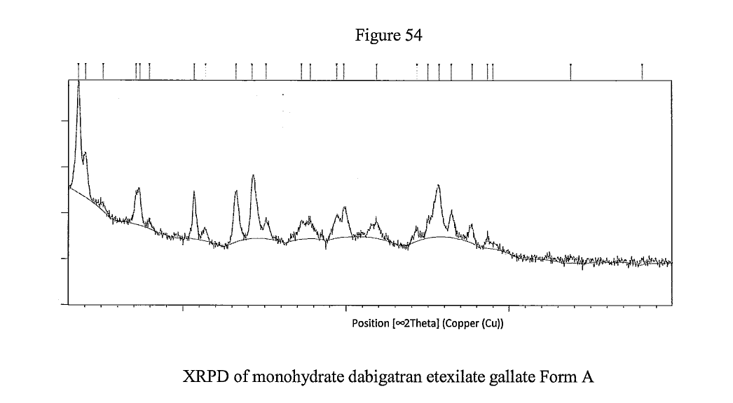

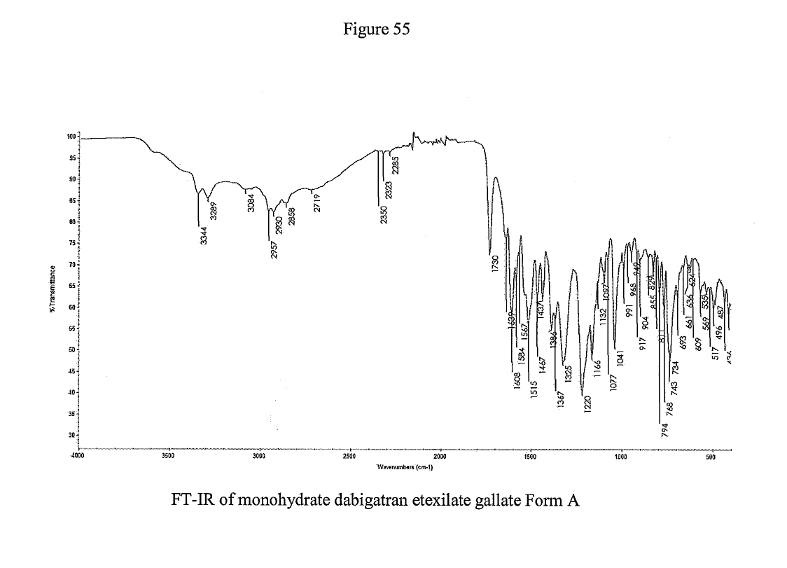

According to a preferred aspect, the invention relates to monohydrate dabigatran etexilate gallate Form A (ratio dabigatran/gallate 1/1), obtained by precipitation shows that the pattern of X-ray diffraction of FIG. 54, the FT-IR spectrum of FIG. 55, and the following characteristics of X-ray diffraction:

| Pos. | Height | FWHM | d---spacing | Rel. Int. |
|---|---|---|---|---|
| [°2Th.] | [cts] | [°2Th.] | [Å] | [%] |
| 3.6078 | 1829.72 | 0.1506 | 24.49073 | 100.00 |
| 4.0403 | 542.18 | 0.1171 | 21.86997 | 29.63 |
| 5.1009 | 72.08 | 0.2007 | 17.32487 | 3.94 |
| 7.1141 | 286.34 | 0.1338 | 12.42595 | 15.65 |
| 7.3643 | 311.14 | 0.1171 | 12.00435 | 17.00 |
| 7.9345 | 58.26 | 0.2676 | 11.14288 | 3.18 |
| 10.6881 | 380.86 | 0.0836 | 8.27757 | 20.82 |
| 11.3816 | 81.15 | 0.2676 | 7.77467 | 4.44 |
| 13.2381 | 407.93 | 0.3011 | 6.68824 | 22.29 |
| 14.2161 | 482.83 | 0.1673 | 6.23027 | 26.39 |
| 15.0720 | 145.92 | 0.1004 | 5.87833 | 7.97 |
| 17.2347 | 130.25 | 0.1673 | 5.14524 | 7.12 |
| 17.7980 | 126.32 | 0.2676 | 4.98366 | 6.90 |
| 19.4159 | 162.00 | 0.2676 | 4.57188 | 8.85 |
| 19.8410 | 231.61 | 0.1338 | 4.47487 | 12.66 |
| 21.8579 | 124.24 | 0.1673 | 4.06631 | 6.79 |
| 24.3195 | 72.06 | 0.1673 | 3.66001 | 3.94 |
| 24.9881 | 122.36 | 0.1338 | 3.56357 | 6.69 |
| 25.6717 | 446.68 | 0.2342 | 3.47022 | 24.41 |
| 26.4153 | 189.22 | 0.2342 | 3.37418 | 10.34 |
| 27.7083 | 113.95 | 0.2676 | 3.21960 | 6.23 |
| 28.6667 | 65.02 | 0.2007 | 3.11410 | 3.55 |
| 29.0123 | 32.99 | 0.5353 | 3.07779 | 1.80 |
| 33.7797 | 23.84 | 0.5353 | 2.65353 | 1.30 |
| 38.1664 | 22.49 | 0.2342 | 2.35803 | 1.23 |

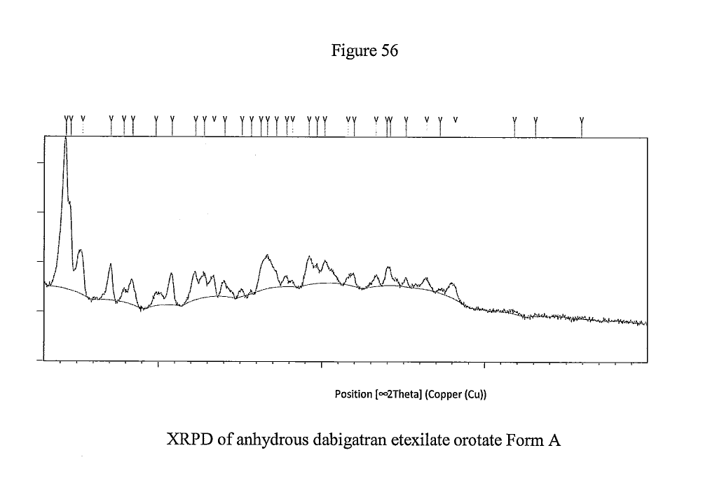

According to another of its aspects, the invention relates to anhydrous dabigatran etexilate orotate (ratio dabigatran/orotate 1/1) obtained by precipitation which shows that the pattern of X-ray diffraction of FIG. 56, the FT-IR spectrum of

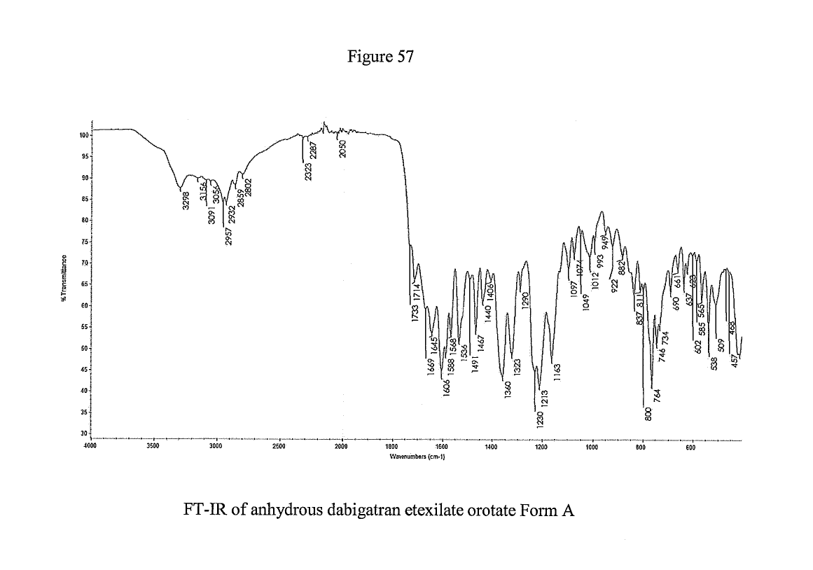

FIG. 57, and the following characteristics of X-ray diffraction:

| Pos. | Height | FWHM | d---spacing | Rel. Int. |
|---|---|---|---|---|
| [°2Th.] | [cts] | [°2Th.] | [Å] | [%] |
| 4.3254 | 6936.74 | 0.1506 | 20.42927 | 100.00 |
| 4.6241 | 2931.79 | 0.1338 | 19.10994 | 42.26 |
| 5.3422 | 1077.47 | 0.1840 | 16.54293 | 15.53 |
| 7.0676 | 932.79 | 0.0836 | 12.50762 | 13.45 |
| 7.8630 | 265.63 | 0.1673 | 11.24408 | 3.83 |
| 8.4018 | 556.70 | 0.3346 | 10.52416 | 8.03 |
| 9.8344 | 248.40 | 0.2342 | 8.99407 | 3.58 |
| 10.8091 | 729.34 | 0.3011 | 8.18517 | 10.51 |
| 12.2591 | 684.33 | 0.0836 | 7.22005 | 9.87 |
| 12.7985 | 594.20 | 0.2007 | 6.91695 | 8.57 |
| 13.3780 | 491.25 | 0.3011 | 6.61860 | 7.08 |
| 14.0497 | 352.00 | 0.2342 | 6.30366 | 5.07 |
| 15.1084 | 145.87 | 0.3011 | 5.86423 | 2.10 |
| 15.6767 | 50.07 | 0.2007 | 5.65292 | 0.72 |
| 16.2470 | 602.87 | 0.2342 | 5.45575 | 8.69 |
| 16.6558 | 986.70 | 0.1338 | 5.32275 | 14.22 |
| 17.2098 | 416.99 | 0.1004 | 5.15263 | 6.01 |
| 17.8344 | 273.39 | 0.2007 | 4.97355 | 3.94 |
| 18.1952 | 146.83 | 0.2007 | 4.87575 | 2.12 |
| 19.2106 | 838.17 | 0.2342 | 4.62026 | 12.08 |
| 19.6955 | 550.55 | 0.1004 | 4.50760 | 7.94 |
| 20.1563 | 622.21 | 0.2342 | 4.40558 | 8.97 |
| 21.5919 | 207.72 | 0.2007 | 4.11580 | 2.99 |
| 21.9700 | 312.58 | 0.2007 | 4.04581 | 4.51 |
| 23.3022 | 274.72 | 0.1338 | 3.81743 | 3.96 |
| 23.9557 | 464.77 | 0.1171 | 3.71476 | 6.70 |
| 24.1604 | 512.68 | 0.1338 | 3.68375 | 7.39 |
| 25.1262 | 245.41 | 0.1673 | 3.54430 | 3.54 |
| 26.4109 | 279.86 | 0.3011 | 3.37474 | 4.03 |
| 27.2296 | 91.31 | 0.3346 | 3.27511 | 1.32 |
| 28.1810 | 291.97 | 0.2007 | 3.16666 | 4.21 |
| 31.8241 | 49.50 | 0.6691 | 2.81198 | 0.71 |
| 33.1134 | 26.99 | 0.6691 | 2.70538 | 0.39 |
| 35.9337 | 18.38 | 0.4015 | 2.49925 | 0.26 |

The new crystalline compounds of the invention, including dabigatran etexilate caffeate forms A and B as defined above, represent another subject matter of the invention.

Details of the two procedures are provided below.

The new crystalline compounds of the invention, including dabigatran etexilate caffeate hydrate as defined above, can be prepared for example by precipitation or by exposure to solvent vapors, technique known as “vapor digestion”.

According to the precipitation technique, a mixture of dabigatran etexilate and the co-former are stirred in a suitable solvent, preferably at room temperature, until the formation of a crystalline compound. If necessary, the solution may be initially heated and/or concentrated. The crystalline compound is subsequently isolated by filtration and optionally washed with a solvent and/or dried, according to the methods known in the art.

The vapor digestion technique, involve the mixing/grinding a solid mixture of dabigatran etexilate with the co-former, exposing the solid mixture to the vapor of a suitable solvent and possibly dried. This technique is therefore not usable with a co-former which is not solid.

According to another of its aspects, the invention relates to a process for the preparation of a crystalline compound according to the invention, or a hydrate or a solvate of such a crystalline compound, which comprises the following steps:

a. dissolving dabigatran etexilate in a suitable solvent and adding the co-former acid;

b. optionally concentrating and/or heating the mixture of step (a);

c. stirring the mixture at room temperature until the formation of the crystalline compound; and

d. isolating the crystalline compound and optionally washing and/or drying the crystalline compound so obtained.

Suitable solvents for the above described process are, for example, esters such as ethyl acetate, ketones such as acetone, chlorinated solvents such as dichloromethane; mixtures of solvents may also be used.

The preferred solvents for the formation of crystalline compounds of the invention with various co-former with the precipitation process are shown in Table (I) below

TABLE I

| | Code of the | |
|---|---|---|
| co-former acid | co-former | solvent |
| trans-aconitic acid | ACO | acetone |
| Adipic acid | ADI | acetone |
| Caffeic acid | CAF | acetone |
| p-cumaric acid | COU | acetone |
| D-gluconic acid | GLUC | acetone |
| α-cheto-glutaric acid | KGL | acetone |
| hippuric acid | HIP | acetone |
| Itaconic acid | ITA | acetone |
| Orotic acid | ORA | acetone |
| piruvic acid | PRV | acetone |
| Sulfamic acid | SUL | acetone |
| D-(−)-quinic acid | QUI | acetone |
| Sebacic acid | SEB | ethyl acetate |
| Gallic acid | GLC | dichloromethane |

All the steps of the process are advantageously carried out at room temperature. If necessary it is however possible to heat during step (a) to favor the dissolution of the two starting compounds.

According to a preferred embodiment, a saturated solution of dabigatran etexilate is prepared to which the acid co-former is added, preferably in an amount equal to one equivalent with respect to dabigatran etexilate.

In some case, step (b) can be carried out, to facilitate the precipitation of the crystal. Step (c) is maintained until the formation of the crystalline compound and it may require from several hours to several days.

The crystalline compound obtained is subsequently processed, in step (d) according to the conventional methods, well known to those skilled in the art.

According to another of its aspects, the invention relates to a process for the preparation of a crystalline compound according to the invention, or a hydrate or a solvate of such a crystalline compound, which comprises the following steps:

a′) mixing and grinding dabigatran etexilate and co-former acid;

b′) exposing the solid mixture to vapors of a suitable solvent;

c′) optionally drying the new crystalline compound thus obtained.

As said, the vapor digestion process can be performed only with co-formers which are solid at room temperature. Examples are D-gluconic acid and pyruvic acid.

All steps of the above procedure are advantageously carried out at room temperature. Step (b′) is performed until the formation of the crystalline compound and may last from a few hours, more often, a few days or even a week. The skilled in the art is perfectly able to evaluate the development of the process, by taking samples and analyzing them according to known techniques.

The crystalline compound obtained is then isolated and processed in step (c′) according to the conventional methods well known to those skilled in the art.

For the crystalline compounds prepared from gallic acid and orobic acid, two forms have been synthesized, namely, a form in which the molar ratio dabigatran/acid is 1/1 (Forms A) and a form of which there are more equivalents of acid compared to dabigatran (Forms B).

While not wishing to be bound to any particular theory, the inventors observed that by carrying out the reaction of step (a) and, if necessary the step (b) in solution (homogeneous mixture), the crystalline compound is obtained in a ratio of 1/1, while operating in suspension (heterogeneous mixture) with more equivalents of acid, crystalline compounds with different molar ratio, such as for instance dabigatran/gallate=1/2 and dabigatran/orotate=1/4 are generated.

The vapor digestion technique is preferably applied with a co-former selected from acid, trans-aconitic acid, adipic acid, caffeic acid, p-coumaric acid, α-keto-glutaric acid, hippuric acid, itaconic acid, sulfamic acid, D-(−)-quinic acid, gallic acid, ferulic acid, D-glutaric acid and vanillic acid.

The preferred solvents for the formation of crystalline compounds of the invention with various co-former with the vapor digestion process are shown in Table (II) below

TABLE II

| | Code of the | |
|---|---|---|
| co-former acid | co-former | solvent |
| trans-aconitic acid | ACO | acetone |
| Adipic acid | ADI | acetone |
| Caffeic acid | CAF | acetone |
| p-cumaric acid | COU | acetone |
| α-cheto-glutaric acid | KGL | acetone |
| Ippuric acid | HIP | acetone |
| itaconic acid | ITA | acetone |
| Sulfamic acid | SUL | acetone |
| D-(−)-quinic acid | QUI | acetone |
| Gallic acid | GLC | dichloromethane |
| Ferulic acid | FER | acetone |
| D-glutaric acid | GTR | acetone |
| Vanillic acid | VAN | acetone |

The characterization data of the crystalline compounds of the invention are provided in the Experimental Section and the graphs of X-ray diffraction (XRPD), infrared (IR), differential scanning calorimetry (DSC) of the compounds are shown in the figures attached to the present description.

The TGA and EGA confirmed the presence or the absence of any solvent in the crystals.

The crystalline compounds of the invention showed the excellent chemical-physical properties and therefore represent valid alternatives to the currently available crystalline forms of dabigatran etexilate for administration to humans and/or in the animal.

Moreover, solubility test were carried out, according to the methods described in the Experimental Section that follows, and it was observed that some representative compounds of the invention show an excellent dissolution rate, higher than that of dabigatran etexilate mesylate available on the market. This result is unexpected and surprising and represents a significant technical advance in the pharmaceutical field, because it is known that in a better solubility results in a better bioavailability of the drug.

According to another of its aspects, the invention also relates to a solid pharmaceutical composition that comprises at least one crystalline compound of the invention together with one or more pharmaceutically acceptable carriers or excipients.

The pharmaceutical compositions of the invention are particularly suitable for oral administration.

For the oral administration, said compositions can be in the form of tablets, capsules or granules and are prepared according to conventional methods with pharmaceutically acceptable excipients such as binding agents, bulking agents, lubricants, disintegrants, wetting agents, flavoring agents, etc. Tablets may also be coated by the methods well known in the art.

The compositions of the invention are advantageously in the form of dosage units. Preferably, each dosage unit according to the invention comprises a crystalline compound according to the invention that contains an amount of dabigatran etexilate from 10 to 200 mg, for example from 50 to 150 mg, advantageously from 70 to 120 mg, for example 75 or 110 mg, advantageously with the excipients and conventional additives well known to those skilled in the art. Other dosages may of course be provided depending on the diseases and conditions of the subject to be treated.

Preferred compositions comprise gallate dabigatran etexilate, advantageously in an monohydrate form.

Other particularly preferred compositions are the compositions comprising the orotate dabigatran etexilate, advantageously in the anhydrous form.

According to another of its aspects, the invention relates to crystalline compounds and/or the pharmaceutical compositions of the invention for their use in therapy, in particular in the tromboembolitic therapy, advantageously in the prevention of thromboembolic episodes and in the prevention of stroke and systemic embolism.

The invention also comprises a method of treatment for the prevention of thromboembolic episodes and for the prevention of stroke and systemic embolism which comprises administering, to a subject in need thereof, an effective amount of a crystalline compound of the invention, advantageously in the form of a pharmaceutical composition as defined above.

### EXPERIMENTAL SECTION

Data and analytical details of the crystalline compounds of the invention are provided in the tables below.

| Technique | Result for Dabigatran Etexilate trans-Acotinate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at 126.9° C. |
| | (Onset 115.9° C.) |
| TGA | The TGA profile shows only the degradation of sample |
| | after approx. 120° C. |
| EGA | The EG analysis confirms sample decomposition showing |
| | carbon dioxide evolution. |

| Technique | Result for Dabigatran Etexilate Adipate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic broad double peak |
| | at approx. 94° C. (Onset 82.7° C.) |
| TGA | The TGA profile shows weight loss above 120° C. due to |
| | decomposition. |
| EGA | The EG analysis confirms sample decomposition showing |
| | carbon dioxide and 1-hexanol evolution. |

| Technique | Result for Dabigatran Etexilate trans-Caffeate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak (melting) at |
| | approx. 99.5° C. (Onset) |
| TGA | The TGA profile shows a mass loss at low temperature |
| | (approx. 50° C.) due to imbibition water. Sample |
| | decomposition occurs in correspondence to the melting |
| | (approx. 100° C.). |
| EGA | The EG analysis confirms the water evolution at low |
| | temperature and sample decomposition revealing carbon |
| | dioxide evolution. |

| Technique | Result for Dabigatran Etexilate p-Coumarate |
|---|---|
| XRPD | The evidenced crystalline form is labeled as Form A |
| FT-IR | The infrared spectrum of the form labeled as Form A |
| | confirms the formation of a likely co-crystal |
| DSC | The DSC profile shows an endothermic peak at 57.6° C. |
| | (Onset 54.5° C.) and a peak corresponding to the melting at |
| | 125.9° C. (Onset 115.5° C.) |
| TGA | The TGA profile shows weight loss of 0.9% at approx. 80° C. |
| | while after 120° C. decomposition occurs |
| EGA | The EG analysis evidences that the first thermal event |
| | showed in DSC corresponds to acetone evolution while |
| | sample decomposition is confirmed by carbon dioxide, |
| | 1-hexanol and ethyl acrylate evolution |

| Technique | Result for Dabigatran Etexilate D-Gluconate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at approx. |
| | 107° C. (Onset 98.2° C.) |
| TGA | The TGA shows a desolvation step between 40-120° C. |
| | followed by degradation |
| EGA | The EG analysis evidences the evolution of ethyl acetate |
| | and carbon dioxide |

| Technique | Result for Dabigatran Etexilate α-Ketoglutarate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic double peak with an |
| | onset at 110.7° C. probably associated to a solid-solid |
| | transition followed by melting and decomposition |
| TGA | The weight loss of 23% observed in the TG profile after |
| | 110° C. is connected to sample decomposition |
| EGA | The EG analysis evidences the evolution of carbon dioxide |

| Technique | Result for Dabigatran Etexilate Hippurate |
|---|---|
| XRPD | The evidenced crystalline form is labeled as Form A |
| FT-IR | The infrared spectrum of the form labeled as Form A |
| | confirms the formation of a new species |
| DSC | The DSC profile shows two endothermic events at 57.5° C. |
| | (Onset 52.9° C. - solid-solid transition) and 141.1° C. |
| | (Onset 136.9° C.-melt) |
| TGA | The TGA profile shows a weight loss of approx. 11% at |
| | 140° C. connected to sample decomposition |
| EGA | The EG analysis evidences the evolution of decomposition |
| | product carbon dioxide and 1-hexanol |

| Technique | Result for Dabigatran Etexilate Itaconate |
|---|---|
| XRPD | The evidenced crystalline form is labeled as Form A |
| FT-IR | The infrared spectrum of the form labeled as Form A confirms |
| | the formation of a new species |
| DSC | The DSC profile shows a large endothermic peak at 95.9° C. |
| | (onset 79° C.) and an endothermic event at 113° C. (Onset |
| | 108.6° C.) |
| TGA | The TGA profile shows a weight loss of approx.1% at 50° C. |
| | and decomposition at approx.140° C. |
| EGA | The EG analysis evidences water evolution in correspondence |
| | to the firs weight loss and carbon dioxide and 1-hexanol |
| | connected to sample decomposition |

| Technique | Result for Dabigatran Etexilate Orotate |
|---|---|
| XRPD | The evidenced crystalline form is labeled as Form B |
| FT-IR | The infrared spectrum of the form labeled as Form B confirms |
| | the formation of a new species |
| DSC | The DSC profile shows an endothermic peak at approx. |
| | 102.3° C. (Onset 89.2° C.) |
| TGA | The TGA profile shows a weight loss of 4% at approx. 60° C. |
| | along with 11% at 150° C. due to decomposition. |
| EGA | The EG analysis evidence water evolution in correspondence |
| | of the first thermal event and carbon dioxide and 1-hexanol |
| | evolution connected to the sample decomposition |

| Technique | Result for Dabigatran Etexilate Pyruvate |
|---|---|
| XRPD | The evidenced crystalline form is labeled as Form A |
| FT-IR | The infrared spectrum of the form labeled as Form A confirms |
| | the formation of a new species |
| DSC | The DSC profile shows an endothermic peak at approx. |
| | 113.4° C. (Onset 102.5° C.) |
| TGA | The TGA profile shows a weight loss of 0.9% at approx. |
| | 50° C. along with 23% at 120° C. due to decomposition. |
| EGA | The EG analysis evidence water evolution in correspondence |
| | of the First thermal event and carbon dioxide and 1-hexanol |
| | evolution connected to the sample decomposition |

| Technique | Result for Dabigatran Etexilate Sulfamate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at approx. |
| | 171.2° C. (Onset 167.2° C.) |
| TGA | The TGA profile shows a typical profile of dried compound, |
| | the Weight loss due to decomposition starts after 170° C. |
| EGA | The EG analysis evidences the evolution of decomposition |
| | compounds in correspondence to the weight loss registered in |
| | TG |

| Technique | Result for Dabigatran Etexilate D-(−)-Quinate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at approx. |
| | 161.4° C. (Onset 158.6° C.) |
| TGA | The TGA profile shows a typical profile of dried compound, |
| | the weight loss due to decomposition starts after 170° C. |
| EGA | The EG analysis evidences the evolution of carbon dioxide |
| | and 1-hexanol in correspondence to the weight loss |
| | registered in TG |

| Technique | Result for Dabigatran Etexilate Ferulate |
|---|---|
| XRPD | The evidenced crystalline form is labeled as Form A |
| FT-IR | The infrared spectrum of the form labeled as Form A |
| | confirms the formation of a new species |
| DSC | The DSC profile shows three endothermic events probably |
| | connected to solid-solid transitions (at 82.6° C. and 103° C.) |
| | and melt (at 129.2° C.) |
| TGA | The TGA profile shows a typical profile of dried compound, |
| | the weight loss due to decomposition starts after 140° C. |
| EGA | The EG analysis evidences only carbon dioxide evolution |
| | caused by decomposition |

| Technique | Result for Dabigatran Etexilate Gallate |
|---|---|
| XRPD | The evidenced crystalline form is labeled as Form B |
| FT-IR | The infrared spectrum of the form labeled as Form B |
| | confirms the formation of a new species |
| DSC | The DSC profile shows an endothermic peak at 84.5° C. |
| | (Onset 78.8° C.) |
| TGA | The TGA profile shows a weight loss of 1.9% at approx. |
| | 60° C. and decomposition after 160° C. |
| EGA | The EG analysis evidence water and carbon dioxide |
| | evolution in correspondence of the first thermal event (60° C.) |
| | before complete decomposition |

| Technique | Result for Dabigatran Etexilate Sebacate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at approx. |
| | 122.8° C. (Onset 128.8° C.) |
| TGA | The TGA profile shows a typical profile of dried compound, |
| | the weight loss starts after 150° C. due to decomposition |
| EGA | The EG analysis evidence carbon dioxide evolution during |
| | decomposition |

| Technique | Result for Dabigatran Etexilate Glutarate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at approx. |
| | 98.3° C. (Onset 85.9° C.) |
| TGA | The TGA profile shows a typical profile of dried compound, |
| | the weight loss starts after 150° C. due to decomposition |
| EGA | The EG analysis evidence carbon dioxide evolution during |
| | decomposition |

| Technique | Result for Dabigatran Etexilate Vanillate |
|---|---|
| XRPD | The evidenced crystalline form is labeled as Form A |
| FT-IR | The infrared spectrum of the form labeled as Form A |
| | confirms the formation of a new species |
| DSC | The DSC profile shows an endothermic peak at 43.9° C. |
| | imputable to a desolvation step, while the melt of the product |
| | occurs at 80.0° C. (Onset 68.2° C.) |
| TGA | The TGA profile shows a typical profile of dried compound, |
| | the weight loss starts after 150° C. due to decomposition |
| EGA | The EG analysis evidence carbon dioxide evolution during |
| | decomposition |

| Technique | Result for Dabigatran Etexilate Gallate |
|---|---|
| XRPD | The evidenced crystalline form is labeled as Form A |
| FT-IR | The infrared spectrum of the form labeled as Form A |
| | confirms the formation of a new species |
| Technique | Result for Dabigatran Etexilate Orotate |
| XRPD | The evidenced crystalline form is labeled as Form A |
| FT-IR | The infrared spectrum of the form labeled as Form A |
| | confirms the formation of a new species |

### X-Ray Powder Diffraction (XRPD)

Instrument type: X'Pert PRO PANalytical

The X'Pert PRO X-ray diffraction system basically consists of the following items:
A console which provides the working environment for the X'Pert PRO system; it includes measuring and control electronics using a microprocessor system, and high tension generator.
A ceramic diffraction X-ray tube, mounted onto the goniometer in a tube shield; described herein below.
A goniometer, the central part of the diffractometer; the goniometer is described herein below.
Optical modules for the incident and the diffracted X-ray beam. These modules can be mounted on PreFIX positions on the goniometer's arms.
A sample stage on which to mount a sample so that its characteristics can be measured.

Sample stage is the generic name given to any device onto which a sample is mounted so that it can be measured or analyzed. The sample stage used on X'Pert PRO system is the sample spinner. The purpose of spinning is to bring more crystallites into the diffraction position in order to reduce the influence of particle statistics on the measurements. The spinning rotation speed can be set at 2, 1, ½, ¼, ⅛, and 1/16 revolutions per second.
A detector to measure the intensity of the diffracted X-ray beam; the goniometer is described herein below.

Ceramic diffraction X-ray tubes

General Tube Specifications

Focus type: LFF (Long Fine Focus)

Focus dimensions: 12 mm×0.4 mm

Focus quality: To COCIR specifications

Take-off angle (with no intensity loss over range)

line focus: 0°-12° (also dependent on shutter opening)

point focus 0°-20° (also dependent on shutter opening)

Be window diameter: 14 mm

Be window thickness: 300 μm

Power Characteristics

High power ceramic diffraction X-ray tube with copper anode

Maximum power: 2.2 kW

Maximum high tension: 60 kV

Maximum anode current 55 mA

Advised power settings: 80%-85% of maximum power

Advised standby ratings: 30-40 kV, 10-20 mA

Spectral Purity

Foreign lines measured with a β-filter

at 40 kV relative to the Kα line: On delivery <1%

Increase per 1000 hours of tube life: <1% for tubes with Cu anode

Environmental Conditions

Operating temperature: +5° C. to +40° C.

Storage temperature: −40° C. to +70° C.

Electrical safety: IEC1010-1

Cooling Water Conditions

The cooling water used should not cause corrosions or deposit sediment in the tube. If the water is dirty or contains an unduly high concentration of salts, use of a closed cooling system employing clean, not distilled water, may be necessary.

Quality: Drinking water

Flow: 3.5-5 l/minute

Maximum pressure: 0.8 MPa

Pressure drop at 3.5 l/minute: 0.2+/−0.04 MPa

Max. Temperature: 35° C.

Min. Temperature: Depends on dew point of air

Goniometers X'Pert PRO

X'Pert PRO X-ray diffraction systems are based on the PW3065/6x Goniometer. The goniometer contains the basic axes in X-ray diffractometry: the θ and 2θ axes.

PW3050/60 X'Pert PRO Standard Resolution Goniometer:

Operation mode Horizontal or vertical, θ-θ or θ-2θ mode

Reproducibility 0.0001° 0.001° (with attachments)

Scan speed 0.000001-1.27°/s

Slew speed 12°/s (with attachments)

Minimum step size 0.001°

2θ range −40°-+220°

θ range −15°-+181°

2θ measurement range Dependent on optics, geometry and sample stage

Diffractometer radius 130-240 mm (X'Pert PRO MPD systems); 240 mm is standard setting

Distance goniometer face-diffraction plane 150 mm

RTMS Detector

X'Celerator:

Used with Line focus and point focus

Used in All systems

Radiation type Optimized for Cu radiation

99% linearity range 0-900 kcps overall 0-7000 cps local

Maximum count rate 5000 kcps overall 250 kcps local

Maximum background noise <0.1 cps

Typical energy resolution for Cu Kα radiation 25%

Efficiency for Cu Kα 93%

Detector window size 15 mm parallel to the line focus 9 mm perpendicular to the line focus

Active length 9 mm

(2.2° at 240 mm goniometer radius; 1.6° at 320 mm goniometer radius)

Smallest step size 0.0021° at 240 mm goniometer radius/0.0016° at 320 mm

goniometer radius

Operating modes Scanning mode

### TG Analyses

Instrument type: Mettler Toledo Stare System

Temperature data

Temperature range RT . . . 1100° C.

Temperature accuracy ±1 K

Temperature precision ±0.4 K

Heating rate 0.02 . . . 250 K/min

Cooling time 20 min (1100 . . . 100° C.)

Sample volume ≦100 μL

Special modes

Automation 34 sample positions

TGA-FTIR coupled with Thermo Nicolet 6700 spectrometer

Balance data XP5

Measurement range ≦5 g

Resolution 1.0 μg

Weighing accuracy 0.005%

Weighing precision 0.0025%

Internal ring weights 2

Blank curve reproducibility better than ±10 μg over the whole temperature range

### DSC Analyses

Instrument type: DSC 200 F3 Maia®

Technical Specifications

Temperature range: −170° C. . . . 600° C.

Heating rates: 0.001 K/min . . . 100K/min

Cooling rates 0.001 K/min . . . 100K/min(depending on temperature)

Sensor: heat flux system

Measurement range 0 mW . . . ±600 mW

Temperature accuracy: 0.1 K

Enthalpy accuracy: generally <1%

Cooling options: Forced air (down to RT), LN2 (down to −170° C.) Purge gas rate: 60 ml/min

Intracooler for the extended rate: −40° . . . 600° C.

### FT-IR

Instrument type: Nicolet FT-IR 6700 ThermoFischer

Technical Specifications

Product Specifications

Spectral Range (Standard): 7800-350 cm-1

Spectral Range (Option, CsI Optics): 6400-200 cm-1

Spectral Range (Option, Extended-Range Optics): 11000-375 cm-1

Spectral Range (Option, Multi-Range Optics): 27000-15 cm-1

Optical Resolution: 0.09 cm-1

Peak-To-Peak Noise (1 minute scan): <8.68×10-6 AU*

RMS Noise (1 minute scan): <1.95×10-6 AU*

Ordinate Linearity: 0.07% T

Wavenumber Precision: 0.01 cm-1

Slowest Linear Scan Velocity: 0.158 cm/sec

Fastest Linear Scan Velocity: 6.33 cm/sec

Number of Scan Velocities: 15

Rapid Scan (Spectra/second @ 16 cm-1, 32 cm-1): 65, 95

* AU: Absorbance Units.

Smart Performer

For single-reflection ATR analysis.

Crystal Materials: ZnSe

Sampling Area: 2 mm

Spectral Range: 20000 to 650 cm-1 (ZnSe)

Depth of Penetration: 2.03 micrometers at 1000 cm-1

Refractive Index: 2.4

Useful pH: 5-9

Instrument setup

Number of sample scans: 32

Number of background scans: 32

Resolution: 4,000 cm-1

Sample gain: 8.0

Optical velocity: 0.6329

Aperture: 100.00

Detector: DTGS KBr

Beamsplitter: KBr

### Example 1

### General Preparation of Crystalline Compounds by Precipitation

To a saturated solution of dabigatran etexilate tetrahydrate in the selected solvent, 1 molar equivalent of the acid co-former is added. The mixture is stirred at room temperature and the precipitate is recovered by filtration, washed with a solvent and dried before proceeding with the analysis.

| Example | Co-former | Solvent | Stirring time^{a} | Drying |
|---|---|---|---|---|
| 1.1 | ACO | acetone | 1 hr | air/2 hrs |
| 1.2 | ADI | acetone | 24 hrs | under vacuum/40° C./18 hrs |
| 1.3 | CFA | acetone^{c} | 20 hrs | under vacuum/40° C./18 hrs |
| 1.4 | GLU | acetone | 20 hrs | under vacuum/40° C./18 hrs |
| 1.5 | KGL | acetone | 20 hrs | under vacuum/40° C./18 hrs |
| 1.6 | HIP | Acetone^{c} | 20 hrs | under vacuum/40° C./18 hrs |
| 1.7 | ITA | acetone | 20 hrs | under vacuum/40° C./18 hrs |
| 1.8 | ORA | acetone | 20 hrs | under vacuum/40° C./18 hrs (1/4) |
| 1.9 | ORA | acetone | 20 hrs | under vacuum/40° C./18 hrs (1/1) |
| 1.10 | PRV | acetone | 1 hr | under vacuum/40° C./18 hrs |
| 1.12 | SUL | acetone | 3 hrs | under vacuum/40° C./18 hrs |
| 1.12 | QUI | acetone | 3 hrs | under vacuum/40° C./18 hrs |
| 1.13 | GLC | dichoromethane | 3 days | under vacuum/40° C./18 hrs (1/2) |
| 1.14 | GLC | dichoromethane | 20 hrs | under vacuum/40° C./18 hrs (1/1) |
| 1.15 | SEB | ethyl acetate | 18 hrs^{b} | under vacuum/40° C./18 hrs |
| 1.16 | COU | acetone | 3 days | — |

^{a}stirring at room temperature
^{b}mixture initially heated to 50° C. for 60 minutes before stirring at room temperature
^{c}no precipitate after 3 days; evaporation to air for two days

### Example 2

### General Preparation of Crystalline Compounds by Vapor Digestion

100 mg of dabigatran etexilate tetrahydrate, and 1 molar equivalent of the acid co-former are mixed and homogenized in a mortar with a pestle. The mixture is then exposed to vapors of a solvent at 25° C. The powder is recovered and dried before proceeding with the analysis.

| Example | co-former | solvent | conditions | drying |
|---|---|---|---|---|
| 2.1 | ACO | acetone | 25° C./3 days | under vacuum/40° C./18 hrs |
| 2.2 | ADI | acetone | 25° C./7 days | — |
| 2.3 | CFA | acetone | 25° C./7 days | under vacuum/40° C./18 hrs |
| 2.4 | COU | acetone | 25° C./7 days | under vacuum/40° C./18 hrs |
| 2.5 | KGL | acetone | 25° C./3 days | under vacuum/40° C./18 hrs |
| 2.6 | HIP | acetone | 25° C./3 days | under vacuum/40° C./18 hrs |
| 2.7 | ITA | acetone | 25° C./7 days | under vacuum/40° C./18 hrs |
| 2.8 | SUL | acetone | 25° C./3 days | under vacuum/40° C./18 hrs |
| 2.9 | QUI | acetone | 25° C./3 days | under vacuum/40° C./18 hrs |
| 2.10 | GLC | dichloromethane | 25° C./3 days | under vacuum/40° C./18 hrs |
| 2.11 | FER | acetone | 25° C./6 days | under vacuum/40° C./18 hrs |
| 2.12 | VAN | acetone | 25° C./3 days | under vacuum/40° C./18 hrs |

### Example 3

### Dabigatran Etexilate Gallate Monohydrate Form A (Dabigatran/Gallic Acid 1/1 Mol/Mol)

In a reactor 100 g of dabigatran etexilate and 500 g of acetone are loaded. The slurry is heated at 40° C. until dissolution. A solution of 26.5 g of gallic acid in 100 g of acetone was then added dropwise within 30 minutes. The precipitation was trigged at 25° C. and the slurry was cooled to 20° C. for 16 hours, the solid was then filtered, washed with 100 g of acetone and dried under vacuum at 30° C. for 16 h. Pale yellow solid: 97.6 g. Yield 78.5%.

### Example 4

### Dabigatran Etexilate Gallate Hydrate Form B (Dabigatran/Gallic Acid 1/2 Mol/Mol)

In a 100-mL round bottom flask, equipped with a magnetic stirring bar and a condenser, 1 g of dabigatran etexilate was charged (1.593 mmol). 40 mL of dichloromethane were transferred into the reaction flask and the mixture was stirred at 50° C. until a total dissolution of the starting material was observed. 1 eq. of gallic acid (1.593 mmol=271 mg) was added and the mixture was stirred at 50° C. for 30 minutes but a totally dissolution of the coformer was not achieved. The mixture was slowly cooled at room temperature and stirred for 18 hours. The solid was recovered under vacuum, washed with ethyl acetate (20 mL×2) and dried at 40° C. for 72 hours. 0.66 g of white solid was recovered (Y=52.1%).

### 1H-NMR

1H-NMR (400 MHz, dmso-d6, 25° C.): δ ppm 0.87 (t, J=7.0 Hz, 3H), 1.12 (t, J=7.0 Hz, 3H), 1.26-1.34 (m, 6H), 1.55-1.60 (m, 2H), 2.68 (t, J=7.2 Hz, 2H), 3.76 (s, 3H), 3.94-4.00 (m, 4H), 4.22 (t, J=7.0 Hz, 2H), 4.59 (d, J=5.2 Hz, 2H), 6.76 (d, J=8.8 Hz, 2H), 6.88 (d, J=8.4 Hz, 1H), 6.91 (s, 2H), 6.92 (t, J=5.2 Hz, 1H), 7.10-7.13 (m, 1H), 7.15 (dd, J1=8.0 Hz, J2=1.6 Hz, 1H), 7.40 (d, J=8.4 Hz, 1H), 7.46-7.47 (m, 1H), 7.52-7.56 (m, 1H), 7.79 (d, J=8.8 Hz, 2H), 8.37-8.40 (m, 1H), 8.83 (bb, 2H, NH2), 9.16 (bb, 3H, OH), 12.20 (bb, 1H, COOH).

### Example 5

### Dabigatran Etexilate Orotate Anhydrous Form a (Dabigatran/Orotic Acid 1/1 Mol/Mole)

In a reactor 8.5 g of dabigatran etexilate, 2.6 g of orotic acid and 25 mL of N,N-dimethylformamide are loaded. The mixture is heated at 50° C. until dissolution. The solution is then brought to 35° C. and 125 mL of acetone are added dropwise within 90 minutes. After precipitation, the slurry was cooled to 20° C. for 3 hours, then the solid was filtered, washed with 10 mL of acetone and dried under vacuum at 30° C. for 16 h. White solid: 8.19 g. Yield 82%.

### Example 6

### Dabigatran Etexilate Orotate Hydrate Form B (Dabigatran/Orotic Acid 1/4 Mol/Mol)

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of orotic acid (1.593 mmol=286 mg) was added and a total dissolution was not observed because the orotic acid was not completely soluble in the acetone. During the dissolution of the coformer a simultaneous formation of a white precipitate was observed.

The mixture was stirred at room temperature for 24 hours. The solid was recovered under vacuum, washed with ethyl acetate (20 mL×2) and dried at 40° C. for 72 hours. 0.56 g of white solid were recovered (Y=43.5%).

### 1H-NMR

1H-NMR (400 MHz, dmso-d6, 25° C.): δ ppm 0.87 (t, J=7.0 Hz, 3H), 1.12 (t, J=7.0 Hz, 3H), 1.26-1.38 (m, 6H), 1.60-1.66 (m, 2H), 2.68 (t, J=6.9 Hz, 2H), 3.76 (s, 3H), 3.97 (q, J=7.0 Hz, 2H), 4.16 (t, J=6.9 Hz, 2H), 4.22 (t, J=6.9 Hz, 2H), 4.65 (d, J=4.8 Hz, 2H), 5.94 (d, J=2.0 Hz, 1H), 6.83 (d, J=9.2 Hz, 2H), 6.89 (d, J=8.0 Hz, 1H), 7.10-7.13 (m, 1H), 7.15 (dd, J1=8.0 Hz, J2=1.6 Hz, 1H), 7.40 (d, J=8.8 Hz, 1H), 7.46-7.47 (m, 1H), 7.52-7.56 (m, 1H), 7.69 (d, J=8.8 Hz, 2H), 8.37-8.40 (m, 1H), 9.76 (bb, 2H, NH2), 10.62 (bb, OH), 11.24 (bb, COOH).

### Example 7

### A Pharmaceutical Composition Comprising Dabigatran Etexilate Gallate

A hard gelatine capsule contains:

75 mg of monohydrate dabigatran etexilate orotate;

Ingredients: tartaric acid, gum arabic, hypromellose, dimethicone 350, hydroxypropyl cellulose and talc.

### Example 8

### A Pharmaceutical Composition Comprising Anhydrous Dabigatran Etexilate Gallate

A hard gelatine capsule contains:

75 mg of anhydrous dabigatran etexilate gallate;

Ingredients: tartaric acid, gum arabic, hypromellose, dimethicone 350, hydroxypropyl cellulose and talc.

### Example 9

### Dabigatran Etexilate Aconitate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of aconitic acid (1.593 mmol=277.4 mg) was added and a total dissolution was observed. After few minutes a large amount of white precipitate was formed. The mixture was stirred at room temperature for 3 hours. The solid was recovered under vacuum, washed with ethyl acetate (20 mL×2) and dried at 40° C. for 72 hours. 1.12 g of white solid was recovered (Y=87.7%).

1H-NMR (400 MHz, DMSO-d6, d1=10 sec.) δ: 0.87 (3H, t, J=6.8 Hz), 1.12 (3H, t, J=6.8 Hz), 1.26-1.40 (6H, m), 1.59 (2H, quint, J=6.8 Hz), 2.68 (2H, t, J=6.8 Hz), 3.68 (2H, s), 3.76 (3H, s), 3.95-4.05 (4H, m), 4.22 (2H, t, J=6.8 Hz), 4.60 (2H, d, J=5.6 Hz), 6.70 (1H, s), 6.77 (2H, d, J=8.4 Hz), 6.89 (1H, d, J=7.2 Hz), 7.04 (1H, br. t), 7.10-7.14 (1H, m), 7.16 (1H, dd, J=8.4 Hz, J1,3=1.6 Hz), 7.39 (1H, d, J=8.4 Hz), 7.47 (1H, d, J1,3=1.6 Hz), 7.54 (1H, dt, J=8.0 Hz, J1,3=1.6 Hz), 7.77 (2H, d, J=8.4 Hz), 8.38-8.40 (1H, m).

By 1H-NMR the stoichiometric ratio is 1:1=dabigatran etexilate:Aconitic Acid.

### Example 10

### Dabigatran Etexilate Adipate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of adipic acid (1.593 mmol=233 mg) was added and the mixture was stirred at room temperature for 24 hours but no precipitate was observed. The reaction was allowed to evaporate at room temperature. When the reaction solvent was decreased to approx. 15 mL the formation of white precipitate was observed. The flask was capped and the reaction was stirred for additional 24 hours.

The solid was recovered under vacuum, washed with ethyl acetate (20 mL×2) and dried at 40° C. for 72 hours. 1H-NMR (400 MHz, DMSO-d6, d1=10 sec.) δ: 0.87 (3H, t, J=7.2 Hz), 1.12 (3H, t, J=6.8 Hz), 1.26-1.40 (6H, m), 1.34-1.52 (4H, m), 1.59 (2H, quint, J=6.8 Hz), 2.15-2.24 (4H, m) 2.68 (2H, t, J=6.8 Hz), 3.76 (3H, s), 3.95-4.05 (4H, m), 4.22 (2H, t, J=6.8 Hz), 4.59 (2H, d, J=5.2 Hz), 6.76 (2H, d, J=9.2 Hz), 6.89 (1H, d, J=8.4 Hz), 6.95 (1H, br. t), 7.10-7.14 (1H, m), 7.16 (1H, dd, J=8.0 Hz, J1,3=1.6 Hz), 7.40 (1H, d, J=8.0 Hz), 7.47 (1H, d, J1,3=1.6 Hz), 7.54 (1H, dt, J=8.0 Hz, J1,3=1.6 Hz), 7.77 (2H, d, J=9.2 Hz), 8.38-8.40 (1H, m).

By 1H-NMR the stoichiometric ratio is 1:0.25=dabigatran etexilate:Adipic Acid.

### Example 11

### Dabigatran Etexilate α-Keto-Glutarate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of α-ketoglutaric acid (1.593 mmol=232.7 mg) was added and a total dissolution was observed. After few minutes a large amount of white precipitate was formed. The mixture was stirred at room temperature for 24 hours. The solid was recovered under vacuum, washed with ethyl acetate (20 mL×2) and dried at 40° C. for 72 hours. 1.02 g of white solid were recovered (Y=83%).

1H-NMR (400 MHz, DMSO-d6, d1=10 sec.) δ: 0.87 (3H, t, J=6.8 Hz), 1.12 (3H, t, J=6.8 Hz), 1.20-1.40 (6H, m), 1.59 (2H, quint, J=8.0 Hz), 2.47 (2H, t, J=6.8 Hz), 2.68 (2H, t, J=6.8 Hz), 2.89 (2H, br. t), 3.76 (3H, s), 3.95-4.05 (4H, m), 4.22 (2H, t, J=7.2 Hz), 4.60 (2H, d, J=5.6 Hz), 6.78 (2H, d, J=8.8 Hz), 6.89 (1H, d, J=8.4 Hz), 7.06 (1H, br. t), 7.09-7.14 (1H, m), 7.16 (1H, dd, J=8.0 Hz, J1,3=1.6 Hz), 7.40 (1H, d, J=8.4 Hz), 7.47 (1H, d, J1,3=1.6 Hz), 7.54 (1H, dt, J=8.0 Hz, J1,3=1.6 Hz), 7.77 (2H, d, J=8.8 Hz), 8.38-8.40 (1H, m).

By 1H-NMR the stoichiometric ratio is 1:1=dabigatran etexilate:α-Ketoglutaric Acid.

### Example 12

### Dabigatran Etexilate Ippurate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of hippuric acid (1.593 mmol=285 mg) was added and a total dissolution was observed. After few minutes a large amount of white precipitate was formed. The mixture was stirred at room temperature for 2 hours. The solid was recovered under vacuum, washed with ethyl acetate (20 mL×2) and dried at 40° C. for 72 hours. 0.932 g of product was isolated (Y=72.5%).

1H-NMR (400 MHz, dmso-d6, 25° C.): δ ppm 0.87 (t, J=7.0 Hz, 3H), 1.12 (t, J=7.0 Hz, 3H), 1.26-1.34 (m, 6H), 1.55-1.60 (m, 2H), 2.68 (t, J=7.2 Hz, 2H), 3.76 (s, 3H), 3.92 (d, J=5.6 Hz, 2H), 3.94-4.00 (m, 4H), 4.22 (t, J=7.0 Hz, 2H), 4.59 (d, J=5.2 Hz, 2H), 6.76 (d, J=8.8 Hz, 2H), 6.88 (d, J=8.4 Hz, 1H), 6.95 (t, J=5.4 Hz, 1H), 7.10-7.13 (m, 1H), 7.15 (dd, J1=8.0 Hz, J2=1.6 Hz, 1H), 7.40 (d, J=8.4 Hz, 1H), 7.46-7.57 (m, 2H+2H), 7.79 (d, J=8.8 Hz, 2H), 7.86-7.89 (m, 2H), 8.37-8.40 (m, 1H), 8.82 (t, J=5.6 Hz, 1H)

By 1H-NMR the stoichiometric ratio is 1:1=dabigatran etexilate:Hippuric Acid.

### Example 13

### Dabigatran Etexilate Itaconate Hydrate

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of itaconic acid (1.593 mmol=277.4 mg) was added and the mixture was stirred at room temperature for 24 hours but no precipitate was observed. The reaction was allowed to evaporate at room temperature. When the reaction solvent was decreased to approx. 8 mL the formation of a white precipitate was observed. The flask was capped and the reaction was stirred for additional 24 hours. The solid was recovered under vacuum, washed with ethyl acetate (20 mL×2) and dried at 40° C. for 72 hours.

1H-NMR (400 MHz, DMSO-d6, d1=10 sec.) δ: 0.87 (3H, t, J=6.8 Hz), 1.12 (3H, t, J=7.2 Hz), 1.25-1.38 (6H, m), 1.58 (2H, quint, J=7.6 Hz), 2.68 (2H, t, J=7.2 Hz), 3.20 (2H, s), 3.76 (3H, s), 3.95-4.05 (4H, m), 4.22 (2H, t, J=7.2 Hz), 4.59 (2H, d, J=5.2 Hz), 5.69 (1H, s), 6.09 (1H, s), 6.76 (2H, d, J=9.2 Hz), 6.88 (1H, d, J=8.0 Hz), 6.99 (1H, br. t, J=5.6 Hz), 7.07-7.14 (1H, m), 7.15 (1H, dd, J=8.4 Hz, J1,3=1.6 Hz), 7.40 (1H, d, J=8.4 Hz), 7.47 (1H, s), 7.55 (1H, dt, J=8.0 Hz, J1,3=1.6 Hz), 7.78 (2H, d, J=8.4 Hz), 8.38-8.40 (1H, m).

By 1H-NMR the stoichiometric ratio is 1:1=dabigatran etexilate:Itaconic Acid.

### Example 14

### Dabigatran Etexilate Pyruvate Hydrate

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of Pyruvic Acid (1.593 mmol=113 μL) was added and the mixture was stirred at room temperature for 90 minutes. After few minutes a large amount of white precipitate was formed. The solid was recovered under vacuum, washed with ethyl acetate (20 mL×2) and dried at 40° C. for 72 hours. 0.671 g of white solid was recovered (Y=58.9%).

1H-NMR (400 MHz, dmso-d6, 25° C.): δ ppm 0.87 (t, J=7.2 Hz, 3H), 1.12 (t, J=7.2 Hz, 3H), 1.26-1.34 (m, 6H), 1.55-1.63 (m, 2H), 2.29 (s, 3H), 2.68 (t, J=7.2 Hz, 2H), 3.76 (s, 3H), 3.94-4.05 (m, 4H), 4.22 (t, J=7.2 Hz, 2H), 4.61 (d, J=5.6 Hz, 2H), 6.78 (d, J=8.8 Hz, 2H), 6.89 (d, J=8.4 Hz, 1H), 7.06 (t, J=6.0 Hz, 1H), 7.10-7.13 (m, 1H), 7.15 (dd, J1=8.0 Hz, J2=1.6 Hz, 1H), 7.40 (d, J=8.4 Hz, 1H), 7.47 (dd, 1H, J2=1.6 Hz), 7.54 (dt, J=7.2 Hz, J2=1.6 Hz, 2H), 7.77 (d, J=8.8 Hz, 1H), 8.37-8.40 (m, 1H).

By 1H-NMR the stoichiometric ratio is 1:1=dabigatran etexilate:Pyruvic Acid.

### Example 15

### Dabigatran Etexilate Sulfamate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of sulfamic acid (1.593 mmol=154 mg) was added and a total dissolution was observed. After few minutes a large amount of white precipitate was formed. The mixture was stirred at room temperature for 4 hours. The solid was recovered under vacuum, washed with ethyl acetate (20 mL×2) and dried at 40° C. for 72 hours. 0.81 g of white solid was recovered (Y=70%).

1H-NMR (400 MHz, dmso-d6, 25° C.): δ ppm 0.87 (t, J=6.8 Hz, 3H), 1.12 (t, J=6.8 Hz, 3H), 1.20-1.38 (m, 6H), 1.61 (quint, 2H, J=5.6 Hz), 2.68 (t, 2H, J=7.2 Hz), 3.77 (s, 3H), 3.97 (quart, J=7.2 Hz, 2H), 4.06 (t, 2H, J=6.8 Hz), 4.22 (t, J=7.2 Hz, 2H), 4.62 (d, J=5.6 Hz, 2H), 6.79 (d, J=9.2 Hz, 2H), 6.89 (d, J=8.4 Hz, 1H), 7.09-7.20 (m, 3H), 7.40 (d, J=8.0 Hz, 1H), 7.47 (d, 1H, J1,3=1.6 Hz), 7.54 (dt, 1H, J=8.0 Hz, J1,3=1.6 Hz), 7.74 (d, J=9.2 Hz, 2H), 8.37-8.40 (m, 1H).

### Example 16

### Dabigatran Etexilate D-(−)-Quinate Anhydrous

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of D-(−)-quinic acid (1.593 mmol=277.4 mg) was added and a total dissolution was not observed because the D-(−)-quinic acid was not completely soluble in the acetone. During the dissolution of the coformer a contemporary formation of a yellow precipitate was observed. After few minutes a large amount of yellow precipitate was formed. The mixture was stirred at room temperature for 4 hours. The solid was recovered under vacuum, washed with ethyl acetate (20 mL×2) and dried at 40° C. for 72 hours. 1.11 g of white solid was recovered (Y=84.7%).

1H-NMR (400 MHz, dmso-d6, 25° C.): δ ppm 0.87 (t, J=7.0 Hz, 3H), 1.12 (t, J=7.0 Hz, 3H), 1.26-1.34 (m, 6H), 1.55-1.60 (m, 2H), 1.66-1.78 (m, 2H), 1.83-1.89 (m, 2H), 2.68 (t, J=7.2 Hz, 2H), 3.24-3.27 (m, 1H), 3.71-3.75 (m, 1H), 3.76 (s, 3H), 3.89 (bb, 1H), 3.94-4.00 (m, 4H), 4.22 (t, J=7.0 Hz, 2H), 4.50 (bb, OH), 4.55 (bb, OH), 4.59 (d, J=5.2 Hz, 2H), 6.76 (d, J=8.8 Hz, 2H), 6.88 (d, J=8.4 Hz, 1H), 6.98 (t, J=5.2 Hz, 1H), 7.10-7.13 (m, 1H), 7.15 (dd, J1=8.0 Hz, J2=1.6 Hz, 1H), 7.40 (d, J=8.4 Hz, 1H), 7.46-7.47 (m, 1H), 7.52-7.56 (m, 1H), 7.79 (d, J=8.8 Hz, 2H), 8.37-8.40 (m, 1H).

By 1H-NMR the stoichiometric ratio is 1:1=dabigatran etexilate:D-(−)-Quinic Acid.

### Example 17

### Dabigatran Etexilate Ferulate Anhydrous

1 g (1.593 mmol) of dabigatran etexilate and 1 eq. (309.3 mg) of ferulic acid were homogenized by a pestle in a mortar and then the mixture was exposed to acetone vapor at 25° C. for 3 days. The powder was recovered and dried under vacuum at 40° C. for 48 hours. 1.16 g of white solid was recovered (Y=88.8%).

1H-NMR (400 MHz, dmso-d6, 25° C.): δ ppm 0.87 (t, J=7.0 Hz, 3H), 1.12 (t, J=7.2 Hz, 3H), 1.26-1.38 (m, 6H), 1.54-1.60 (m, 2H), 2.68 (t, J=7.2 Hz, 2H), 3.76 (s, 3H), 3.81 (s, 3H), 3.92-4.02 (m, 4H), 4.22 (t, J=7.2 Hz, 2H), 4.59 (d, J=5.6 Hz, 2H), 4.59 (d, J=5.6 Hz, 2H), 6.36 (d, J=16.4 Hz, 1H), 6.71-6.81 (m, 1H+1H CH═CH), 6.88 (d, J=7.6 Hz, 1H), 6.95 (t, J=5.2 Hz, 1H), 7.05-7.18 (m, 1H+2HAr), 7.27 (d, J2=1.6 Hz, 1H), 7.40 (d, J=8.4 Hz, 1H), 7.47 (d, J2=1.6 Hz, 1H), 7.54 (dt, J=8.0 Hz, J2=1.6 Hz, 1H), 7.79 (d, J=8.4 Hz, 2H), 8.37-8.40 (m, 1H).

By 1H-NMR the stoichiometric ratio is 1:1=dabigatran etexilate:Ferulic Acid.

### Example 18

### Dabigatran Etexilate Glutarate Anhydrous

1 g (1.593 mmol) of dabigatran etexilate and 1 eq. (210.5 mg) of glutaric acid were homogenized by a pestle in a mortar and then the mixture was exposed to acetone vapor at 25° C. for 3 days. The powder was recovered and dried under vacuum at 40° C. for 48 hours. 0.92 g of yellow solid was recovered (Y=76.2%).

1H-NMR (400 MHz, DMSO-d6, d1=10 sec.) δ: 0.87 (3H, t, J=7.2 Hz), 1.12 (3H, t, J=7.2 Hz), 1.26-1.40 (6H, m), 1.56 (2H, quint, J=6.4 Hz), 1.69 (2H, quint, J=7.6 Hz), 2.23 (4H, t, J=7.6 Hz), 2.68 (2H, t, J=6.8 Hz), 3.76 (3H, s), 3.95-4.05 (4H, m), 4.22 (2H, t, J=6.8 Hz), 4.59 (2H, d, J=5.2 Hz), 6.76 (2H, d, J=8.8 Hz), 6.88 (1H, d, J=8.0 Hz), 6.95 (1H, br. t), 7.10-7.14 (1H, m), 7.16 (1H, dd, J=8.4 Hz, J1,3=1.6 Hz), 7.40 (1H, d, J=8.4 Hz), 7.47 (1H, d, J1,3=1.6 Hz), 7.54 (1H, dt, J=8.0 Hz, J1,3=1.6 Hz), 7.79 (2H, d, J=8.8 Hz), 8.38-8.40 (1H, m).

By 1H-NMR the stoichiometric ratio is 1:1=dabigatran etexilate:Glutaric Acid.

### Example 19

### Dabigatran Etexilate Vanillate Hydrate

1 g (1.593 mmol) of dabigatran etexilate and 1 eq. (268 mg) of vanillic acid were homogenized by a pestle in a mortar and then the mixture was exposed to acetone vapor at 25° C. for 3 days. The powder was recovered and dried under vacuum at 40° C. for 48 hours. 0.986 g of white solid was recovered (Y=77.7%).

1H-NMR (400 MHz, dmso-d6, 25° C.): δ ppm 0.87 (t, J=7.0 Hz, 3H), 1.12 (t, J=7.2 Hz, 3H), 1.26-1.38 (m, 6H), 1.54-1.60 (m, 2H), 2.68 (t, J=7.2 Hz, 2H), 3.76 (s, 3H), 3.80 (s, 3H), 3.92-4.02 (m, 4H), 4.22 (t, J=7.2 Hz, 2H), 4.59 (d, J=5.6 Hz, 2H), 6.76 (d, J=8.8 Hz, 2H), 6.83 (d, J=8.4 Hz, 1H), 6.88 (d, J=7.6 Hz, 1H), 6.95 (t, J=5.2 Hz, 1H), 7.10-7.30 (m, 1H), 7.15 (dd, J=8.0 Hz, J2=1.6 Hz, 1H), 7.41 (d, J=78.4 Hz, 1H), 7.40-7.46 (m, 2H), 7.47 (d, J2=1.6 Hz, 1H), 7.54 (dt, J=8.0 Hz, J2=1.6 Hz, 1H), 7.79 (d, J=8.4 Hz, 2H), 8.37-8.40 (m, 1H).

By 1H-NMR the stoichiometric ratio is 1:1=dabigatran etexilate:Vanillic Acid.

### Example 20

### Dabigatran Caffeate Etexilate

In a 50-mL round bottom flask, equipped with a magnetic stirring bar, 1 g of dabigatran etexilate was charged (1.593 mmol). 20 mL of acetone were transferred into the reaction flask and the mixture was stirred at room temperature until a total dissolution of the starting material was observed. 1 eq. of Caffeic Acid (1.593 mmol=287 mg) was added and the mixture was stirred at room temperature for 24 hours but no precipitate was observed. The reaction was allowed to evaporate at room temperature. When the reaction solvent was decreased to approx. 10 mL the formation of white precipitate was observed. The flask was capped and the reaction was stirred for additional 24 hours. The solid was recovered under vacuum, washed with ethyl acetate (20 mL×2) and dried at 40° C. for 72 hours. 928 mg of white solid were recovered (Y=72.1%).

1H-NMR (400 MHz, dmso-d6, 25° C.): δ ppm 0.87 (t, J=7.0 Hz, 3H), 1.12 (t, J=7.0 Hz, 3H), 1.26-1.34 (m, 6H), 1.54-1.60 (m, 2H), 2.68 (t, J=7.2 Hz, 2H), 3.76 (s, 3H), 3.94-4.00 (m, 4H), 4.22 (t, J=7.0 Hz, 2H), 4.59 (d, J=5.2 Hz, 2H), 6.16 (d, J=15.6 Hz, 1H), 6.75 (d, J=8.0 Hz, 1H), 6.76 (d, J=8.8 Hz, 2H), 6.88 (d, J=8.4 Hz, 1H), 6.93-6.97 (m, 1H+1H), 7.02 (d, J=2.0 Hz, 1H), 7.10-7.13 (m, 1H), 7.15 (dd, J1=8.0 Hz, J2=1.6 Hz, 1H), 7.40 (d, J=8.4 Hz, 1H), 7.41 (d, J=15.6 Hz, 1H), 7.46-7.47 (m, 1H), 7.52-7.56 (m, 1H), 7.79 (d, J=8.8 Hz, 2H), 8.37-8.40 (m, 1H).

By 1H-NMR the stoichiometric ratio is 1:1=dabigatran etexilate:Caffeic Acid.

### Solubility Tests

### General Procedures

The solubility tests were performed in a buffer solution at ph 4.5 and compared with the solubility data of dabigatran etexilate mesylate (commercial form). In the HPLC method herein below, acetonitrile was used to dissolve the active ingredient.

### HPLC Method

Instrument: 1200 Infinity Series AGILENT

G4220B—1290 BinPumpVL

G4226A—1290 Sampler

G1316A—1260 TCC

G1314F—1260 VWD

Column: Kinetex 1.7 μm C8 100A, 100×3 mm, Phenomenex

Column Temperature: 30±0.3° C.

Mobile Phase: A: 0.1% Formic Acid in H₂O; B: ACN

Linear Gradient: t=0 A 75%-B 25%
t=4 A 25%-B 75%
t=6 A 0%-B 100%

Post run: 2 min.

Flow: 0.6 mL/min

Pressure initial: 600 bar

Flow Ramp up: 100 mL/min²

Flow Ramp down: 100 mL/min²

Jet Weaver: V100 Mixer

Detector Wavelength: 210 nm

Peakwidth: >0.0031 min (0.63 s resp. Time) (80 Hz)

Injection volume: 3 μl

Injection with needle ash: 3.0 sec.

Stop analysis: 7 min

Retention time: 2.62 min

Diluent: H₂O+0.1% Formic Acid/ACN=6/4

### Thermodynamic Solubility Tests

The sample (approx. 50 mg) was weighted in a vial and left under magnetic stirring (approx. 300 rpm) in approx. 2 mL of buffer solution at 37° C. for 24 hours. The experiments were carried out at pH 4.5 and pH 6.8. The suspensions were filtered with 0.45 μm filter and analyzed by HPLC method previously reported. From the obtained area an opportune dilution of the sample was performed to obtain a value consistent with the Calibration Curve. Every diluted sample was analyzed by HPLC and the results were interpolated by the calibration curve.

Each experiment was replicated twice.

| Sample | Average conc (μg/mL) |
|---|---|
| Mesylate pH 4.5 | 0.28 |
| Gallate pH 4.5 | 0.40 |

### Kinetic Dissolution

### Experimental Conditions for Tablet Dissolution

Dissolution Medium: Phosphate Buffer pH 4.5

Temperature: 37±0.5° C.

Volume: 80 mL

Time: 2 hrs

Sample: Tablet (weight 200 mg)

Stirring: Paddle 100 rpm

Sampling time: 5 min, 15 min, 25 min, 35 min, 45 min, 60 min and 120 min.

Repetitions: 2 for each experiment

At the time fixed, withdraw 3 mL from each vessel. Reinstate the withdrawn volume.

Filter each solution with 0.20 μm filter, discarding the first 1 mL.

### Preparation of the Tablet

A 13 mm tablet with 100 mg of the compound was prepared by a Digital Hydraulic Press (force applied approx 8 metric tons).

### Preparation of the Sample

Each withdrawal was analyzed without further dilution.

### Chromatographic Conditions

The sample was analyzed using the chromatographic conditions reported herein.

| | |
|---|---|
| Time (min) | Average Conc. |

Dabigatran/mesylate 1/1 pH 4.5

| | |
|---|---|
| 5 | 6.86 |
| 15 | 9.09 |
| 25 | 11.40 |
| 35 | 13.28 |
| 45 | 15.96 |
| 60 | 18.55 |
| 120 | 26.44 |

Dabigatran/orotate 1/1 pH 4.5

| | |
|---|---|
| 5 | 8.76 |
| 15 | 11.06 |
| 25 | 12.26 |
| 35 | 13.74 |
| 45 | 15.52 |
| 60 | 17.21 |
| 120 | 28.89 |

Dabigatran/gallate 1/1 pH 4.5

| | |
|---|---|
| 5 | 10.01 |
| 15 | 28.10 |
| 25 | 44.94 |
| 35 | 62.45 |
| 45 | 75.18 |
| 60 | 90.21 |
| 120 | 106.50 |

As it can be seen from the above results, dabigatran etexilate orotate showed an unexpected high thermodynamic solubility, which is more than 1.4 times higher than the mesylate derivative.

Also in the kinetic dissolution test, dabigatran etexilate orotate showed a very high dissolution rate, which is more than 8.7 times higher than the mesylate derivative.

Also the orotate derivative showed an interesting dissolution rate which is comparable with respect to the mesylate salt.

1. A crystalline compound which comprises a mixture of dabigatran etexilate and a monocarboxylic acid selected from gallic acid, orotic acid, p-coumaric acid, hippuric acid, ferulic acid, vanillic acid, hydrates and solvates thereof.

2. The crystalline compound according to claim 1, which is dabigatran etexilate gallate, hydrates and solvates thereof.

3. The crystalline compound according to claim 2, which comprises dabigatran etexilate gallate in a 1/1 molar ratio, hydrates and solvates thereof.

4. The crystalline compound according to claim 3, which is dabigatran etexilate gallate monohydrate showing the X-ray diffraction pattern of FIG. 54 and the FT-IT spectrum of FIG. 55.

5. The crystalline compound according to claim 1, which comprises dabigatran etexilate orotate, hydrates and solvates thereof.

6. The crystalline compound according to claim 5, which comprises dabigatran etexilate orotate in a 1/1 molar ratio, hydrates and solvates thereof.

7. The crystalline compound according to claim 6, which is dabigatran etexilate orotate anhydrous showing the X-ray diffraction pattern of FIG. 56 and the FT-IT spectrum of FIG. 57.

8. A process for the preparation of a crystalline compound according to claim 1, or a hydrate or solvate of such a crystalline compound, which comprises the following steps:
a. dissolving dabigatran etexilate in a suitable solvent and add said monocarboxylic acid;
b. optionally concentrating and/or heating the mixture of step (a);
c. stirring the mixture at room temperature until the crystalline compound is formed; and
d. isolating and optionally washing and/or drying the crystalline compound thus obtained.

9. The process according to claim 8, wherein step (a) involves the use of dabigatran etexilate tetrahydrate.

10. A process for the preparation of a crystalline compound according to claim 2, or a hydrate or solvate of such a crystalline compound, which comprises the following steps:
a″. mixing and grinding dabigatran etexilate and gallic acid;
b″. exposing the solid mixture to vapours of a suitable solvent;
c″. optionally drying the new crystalline compound thus obtained.

11. The process according to claim 10, wherein step (a) involves the use of dabigatran etexilate tetrahydrate.

12. The process according to claim 10, wherein said solvent is dichloromethane.

13. A pharmaceutical composition which comprises a crystalline compound according to claim 1 as the active ingredient, and at least one pharmaceutically acceptable carrier or excipient.

14. A pharmaceutical composition which comprises a crystalline compound according to claim 2 as the active ingredient, and at least one pharmaceutically acceptable carrier or excipient.

15. A crystalline compound according to claim 1, for use in therapy.

16. A crystalline compound according to claim 1, for use in the prevention of thromboembolic events and in the prevention of stroke and systemic embolism.

17. A pharmaceutical composition according to claim 13, for use in therapy.

18. A pharmaceutical composition according to claim 13, for use in the prevention of thromboembolic events and in the prevention of stroke and systemic embolism.
